(19) United States
(12) Patent Application Publication
(10) Pub. No.: US 2008/0153810 A1
(43) Pub. Date: Jun. 26, 2008

(54) INDAZOLE DERIVATIVES USEFUL AS MELANIN CONCENTRATING RECEPTOR LIGANDS

(75) Inventors: Matthew Ronsheim, Port Jefferson, NY (US); Gian-Luca Araldi, East Setauket, NY (US)

Correspondence Address: Forest Laboratories, Inc.;Attn: Charles S. Ryan, 909 3rd Avenue, New York, NY, 10022, US

(73) Assignee: Forest Laboratories Holdings Limited, Hamilton (BM)

(21) Appl. No.: 11/939,565

(22) Filed: Nov. 14, 2007

Related U.S. Application Data
(60) Provisional application No. 60/866,048, filed on Nov. 15, 2006.

(51) Int. Cl. A61K 31/55; C07D 403/04; A61K 31/497; A61P 25/22; A61P 3/04; A61K 31/454; C07D 403/12

(52) U.S. Cl. 514/217.09; 540/603; 544/371; 546/211; 514/254.06; 514/322

(57) ABSTRACT

The present invention relates to novel compounds, in particular, novel indazole that may be used as melanin concentrating hormone receptor ligands, methods of preparing such compounds, compositions containing such compounds, and methods of using such compounds to treat MCH related disorders.

This application claims the benefit of U.S. Provisional Application Ser. No. 60/866,048, filed Nov. 15, 2006, the entire disclosure of which is hereby incorporated by reference.

### FIELD OF THE INVENTION

The present invention relates generally to the field of ligands for melanin concentrating hormone receptors (MCHs), activation of MCHs, and the treatment of disease conditions that respond to MCHs. The present invention relates to novel compounds, in particular, novel indazole that may be used as melanin concentrating hormone receptor ligands, methods of preparing such compounds, compositions containing such compounds, and methods of using such compounds to treat MCH related disorders.

### BACKGROUND OF THE INVENTION

Melanin concentrating hormone (MCH) is a cyclic, 19-amino acid hypothalamic neuropeptide. MCH shows pharmacological activity through modulation of MCH receptors in the central nervous system (CNS). At least two types of MCH receptors are known: type 1 receptors (MCH-1 or SLC-1) and type 2 receptors (MCH-2 or SLT).

MCH plays an important role in the complex regulation of energy balance and body weight. MCH has been found to effect eating behavior and body weight regulation in mammals. In addition, MCH has been shown to play an important role in the regulation of the central nervous system. MCH antagonists have potential to be effective in the treatment of patients with depression and/or anxiety disorders.

Accordingly, there is a need for novel compounds that may be used as MCH receptor ligands for the treatment of MCH related disorders such as, e.g., eating disorders, weight gain, obesity, depression and anxiety.

### SUMMARY OF THE INVENTION

The present invention relates to novel compounds which act as ligands for melanin concentrating hormone receptors, especially compounds which act as ligands for melanin concentrating hormone type-1 receptor (MCH-1).

Still further, the present invention provides methods for synthesizing compounds with such activity and selectivity, as well as methods of (and corresponding pharmaceutical compositions for) treating patients, e.g., mammals, including humans, having a condition that responds to a melanin concentrating hormone receptor ligand, such as MCH-1.

Other and further features and advantages of the present invention will be readily apparent to those skilled in the art upon a reading of the description of the embodiments which follow.

### DETAILED DESCRIPTION OF THE INVENTION

In one aspect, the present invention includes compounds of formula I:

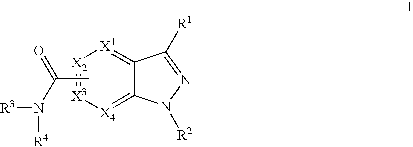

wherein
X¹ to X⁴ are each, independently, N, CH, CR, or C—, wherein C— represents the point of attachment of group —C(O)NR³R⁴;
R is alkyl, alkenyl, alkynyl, cycloalkyl, alkylcycloalkyl, aryl, alkylaryl, heteroaryl, alkylheteroaryl, heterocycle, alkylheterocycle, halogen, hydroxyl, cyano, alkoxy, arylaoxy, cycloalkyloxy, alkoxycarbonyl, carboxyl, amino, alkylamino, dialkylamino, —SH, thioalkyl, alkylsulfonyl, alkylsulfinyl, arylsulfonyl, arylsulfinyl, aminosulfonyl, aminosulfinyl, acyl or aroyl;
R¹ is heterocycle, heteroaryl, or NR^{a}R^{b} where
R^{a} and R^{b} are independently H, alkyl, alkenyl, alkynyl, aminoalkyl, amidoalkyl, cycloalkyl, alkylcycloalkyl, aryl, alkylaryl, heteroaryl, alkylheteroaryl, heterocycle, alkylheterocycle, alkylsulfonyl, or arylsulfonyl, with the proviso that R^{a} and R^{b} and not both H,
R² is H, alkyl, alkenyl, alkynyl, cycloalkyl, alkylcycloalkyl, aryl, alkylaryl, heteroaryl, alkylheteroaryl, heterocycle, alkylheterocycle, aroyl or acyl;
R³ is H, alkyl, alkenyl, alkynyl, cycloalkyl, alkylcycloalkyl, aryl, alkylaryl, heteroaryl, alkylheteroaryl, heterocycle, or alkylheterocycle;
R⁴ is aryl, heteroaryl, heterocycle, alkylaryl, alkylheteroaryl, alkylheterocycle, aryl-X, heteroaryl-X, heterocycle-X, alkylaryl-X, alkylheteroaryl-X, or alkylheterocycle-X; wherein X is Y-alkyl, Y-aryl, Y-alkylaryl, Y-heteoraryl, Y-alkylheteoraryl, Y-heterocycle, or Y-alkylheterocycle, and Y is —O—, —NR⁹—, or —(CO)—;

and pharmaceutically acceptable salts or solvates (e.g., hydrates), or solvates of pharmaceutically acceptable salts thereof,

with the provisos that:
(i) R¹ is not substituted or unsubstituted benzimidazolyl, benzofuranyl or benzothienyl;
(ii) X¹ and X³ are not both N when the —C(O)NR³R⁴ group is attached at the 7-position; and
(iii) said compound is not:
   N-1-azabicycle[2.2.2]oct-3-yl-3-(3-thienyl)-1H-indazole-6 carboxamide,
   3-(2-quinolinyl)-N-tetrahydro-2-oxo-3-furanyl-1H-indazole-5-carboxamide,
   N-(1,3-bendioxol-5-ylmethyl)-3-(3-pyridinyl)-1H-indazole-5-carboxamide,
   N-((3,4-dihydro-2H-1-benzopyran-2-yl)methyl)-3-(2-pyridinyl)-1H-indazole-5-carbosamide,
   3-(5-acetyl-2-thienyl)-N-(3-pyridinylmethyl)-1H-indazole-5-carboxamide,
   N-[2-(1H-indol-3yl)ethyl]-3(4-quinolyl)-1H-indazole-5-carboxamide, or
   N-[[4-(methylsulfonyl)phenyl]methyl]-3-(2-quinolinyl)-1H-indazole-5-carboxamide,

or a pharmaceutically acceptable salt thereof.

According to a further embodiments of the compounds of Formula I, the C(O)NR³R⁴ group is attached at the 4-position. In another embodiment, the —C(O)NR³R⁴ group is attached at the 5-position. In yet another embodiment, the —C(O)NR³R⁴ group is attached at the 6-position. In a further embodiment, the —C(O)NR³R⁴ group is attached at the 7-position. In yet a further embodiment, the C(O)NR³R⁴ group is attached at the 5-position or the 6-position.

In one embodiment, X¹, X² and X⁴ are CH and X³ is —C(O)NR³R⁴. In another embodiment, X¹, X³ and X⁴ are CH and X² is —C(O)NR³R⁴. In a further embodiment, X¹ and X⁴ are CH, one of X² and X³ is CH and the other of X² and X³ is —C(O)NR³R⁴.

In other embodiments, one of X¹-X⁴ is N (e.g., X¹ and X³ are CH or CR, X⁴ is N, and X² is C—; X¹ and X² are CH or CR, X³ is C—, and X⁴ is N; X¹ is N, X² is C—, and X³ and X⁴ are CH or CR; X¹ is N, X² and X⁴ are CH or CR and X³ is C—). According to an additional embodiment, each of X¹ to X⁴ that is not substituted by the —C(O)NR³R⁴ group is CH or CR. According to a further embodiment, each of X¹ to X⁴ that is not substituted by the —CONR³R⁴ group is CH.

In a further embodiment of the compounds of formula I, R¹ is a 5 or 6-membered heterocycle (e.g., substituted or unsubstituted piperazinyl, homopiperazinyl, morpholinyl, pyrrolidinyl (e.g., 4-methyl-piperazin-1-yl, 4-methylsulfonyl-piperazin-1-yl, 4-methyl-homopiperazin-1-yl), a 5 or 6-membered heteroaryl (e.g., substituted or unsubstituted oxazolyl, imidazolyl; triazolyl, tetrazolyl, pyridinyl, pyrimidinyl), or NR^{a}R^{b}. For example, R¹ is substituted or unsubstituted piperazinyl (e.g., 4-methylpiperazinyl) or NR^{a}R^{b} (e.g., N(CH₃)(aminoalkyl). As a further example, R¹ is —NHCH₂CH₂NMe₂, —N(CH₃)CH₂CH₂NMe₂, —NHCH₂CH₂(piperidine), —NHCH₂CH₂CH₂NMe₂, —N(CH₃)CH₂C(O)NHMe, —N(CH₃)CH₂C(O)NMe₂, 4-methylpiperazinyl, 4-methylhomopiperazinyl, 4-methylsulfonylpiperazinyl, oxazolyl, imidazolyl, oxazolylmethyl or thiazolylmethyl. In certain embodiments, R¹ is 4-methylpiperazinyl or —N(CH₃)CH₂CH₂NMe₂.

In each case, when R¹ is heterocycle, the heterocycle may optionally be bridged by a carbon chain linker (e.g., a 1 or 2 carbon atom linker) so as to form a bicyclic group

According to a further embodiment, R^{a} and R^{b} are independently H, alkyl (e.g., methyl), aminoalkyl (e.g., dimethylaminoethyl, dimethylaminopropyl), amidoalkyl (e.g., CH₂CONH₂, CH₂CONHalkyl (e.g., CH₂CONHCH₃) CH₂CONH(alkyl)₂ (e.g., CH₂CON(CH₃)₂), alkylheteroaryl (e.g., thiazolylmethyl, oxazolylmethyl), alkylheterocycle (e.g., piperidinylethyl), alkylsulfonyl (e.g., methylsulfonyl), with the proviso that R^{a} and R^{b} and not both H. For example, one of R^{a} and R^{b} is hydrogen or alkyl (e.g., methyl) and the other of R^{a} and R^{b} is aminoalkyl (e.g., dimethylaminoethyl), amidoalkyl (e.g., CH₂CONH₂, CH₂CONHalkyl (e.g., CH₂CONHCH₃) CH₂CONH(alkyl)₂ (e.g., CH₂CON(CH₃)₂), alkylheteroaryl (e.g., thiazolylmethyl, oxazolylmethyl), alkylheterocycle (e.g., piperidinylethyl), or alkylsulfonyl (e.g., methylsulfonyl). As a further example, one of R^{a} and R^{b} is hydrogen or alkyl (e.g., methyl) and the other is aminoalkyl (e.g., dimethylaminoethyl).

In another embodiment R² is H, alkyl, cycloalkyl, or alkylcycloalkyl. For example, R² is H or alkyl (e.g., methyl). In certain embodiments, R² is H.

In another embodiment R³ is H, alkyl, cycloalkyl, or alkylcycloalkyl. For example, R³ is H or alkyl (e.g., methyl). In certain embodiments, R³ is methyl. In further embodiments, R³ is H.

According to a further embodiment, R⁴ is aryl, heteroaryl, alkylaryl, alkylheteoraryl, aryl-X, heteroaryl-X, alkylaryl-X or alkylheteroaryl-X. In certain embodiments X is Y-aryl or Y-alkylaryl where Y is —O—. For example, R⁴ is aryl (e.g., biphenyl), alkylaryl (e.g., benzyl, substituted benzyl, e.g., chlorobenzyl, methoxybenzyl, trifluoromethylbenzyl, dichlorobenzyl, trifluoromethoxybenzyl), aryl-X or alkylaryl-X where X is Y-alkylaryl (e.g., R⁴ is aryl-Y-alkylaryl (e.g., aryl-O-alkylaryl, such as benzyloxyphenyl), or alkylaryl-Y-alkylaryl (e.g., alkylaryl-O-alkylaryl, such as benzyloxybenzyl).

According to another embodiment, the present invention includes compounds of formula I wherein:

R¹ is heterocycle, heteroaryl other than benzimidazolyl, benzofuranyl, benzothienyl, pyridinyl or quinolinyl, or NR^{a}R^{b};

X¹-X⁴ are CH, CR or C— wherein C— represent the point of attachment of group —C(O)NR³R⁴; and

R⁴ is aryl, heteroaryl, alkylaryl, aryl-X, heteroaryl-X, heterocycle-X, alkylaryl-X, alkylheteroaryl-X, or alkylheterocycle-X.

In certain embodiments, the present invention includes compounds of formula I:

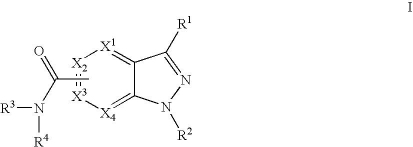

wherein

X¹ and X⁴ are CH;

X² and X³ are each, independently, CH or C—, wherein C— represents the point of attachment of group —(CO)NR³R⁴;

R¹ is heterocycle or NR^{a}R^{b} where

R^{a} and R^{b} are independently H, alkyl or aminoalkyl, with the proviso that R^{a} and R^{b} are not both H;

R² is H or alkyl;

R³ is H;

R⁴ is aryl, alkylaryl, or aryl-X, where X is Y-alkylaryl and Y is —O—;

and pharmaceutically acceptable salts or solvates (e.g., hydrates), or solvates of pharmaceutically acceptable salts thereof.

According to a compound and/or method aspect of the invention, the compound of formula I is chosen from:
3-(4-Methyl-piperazin-1-yl)-1H-indazole-6-carboxylic acid 4-chloro-benzylamide,
3-(4-Methyl-piperazin-1-yl)-1H-indazole-6-carboxylic acid benzylamide,
3-(4-Methyl-piperazin-1-yl)-1H-indazole-6-carboxylic acid 2-methoxy-benzylamide,
3-(4-Methyl-piperazin-1-yl)-1H-indazole-6-carboxylic acid 4-methoxy-benzylamide,
3-(4-Methyl-piperazin-1-yl)-1H-indazole-6-carboxylic acid 4-trifluoromethyl-benzylamide,
3-(4-Methyl-piperazin-1-yl)-1H-indazole-6-carboxylic acid 2,4-dichloro-benzylamide,
3-(4-Methyl-piperazin-1-yl)-1H-indazole-6-carboxylic acid 4-trifluoromethoxy-benzylamide,
3-(4-Methyl-piperazin-1-yl)-1H-indazole-6-carboxylic acid biphenyl-4-ylamide,
3-(4-Methyl-piperazin-1-yl)-1H-indazole-6-carboxylic acid (4-benzyloxy-phenyl)-amide,
1-Methyl-3-(4-methyl-piperazin-1-yl)-1H-indazole-6-carboxylic acid benzylamide,
1-Methyl-3-(4-methyl-piperazin-1-yl)-1H-indazole-6-carboxylic acid 4-trifluoromethyl-benzylamide,
1-Methyl-3-(4-methyl-piperazin-1-yl)-1H-indazole-6-carboxylic acid 2,4-dichloro-benzylamide,
1-Methyl-3-(4-methyl-piperazin-1-yl)-1H-indazole-6-carboxylic acid 4-methoxy-benzylamide,
1-Methyl-3-(4-methyl-piperazin-1-yl)-1H-indazole-6-carboxylic acid 2-methoxy-benzylamide,
4-Benzyloxy-phenyl)-[1-methyl-3-(4-methyl-piperazin-1-yl)-1H-indazol-6-yl]-methanone,
1-Methyl-3-(4-methyl-piperazin-1-yl)-1H-indazole-6-carboxylic acid 4-trifluoromethoxy-benzylamide,
3-[(2-Dimethylamino-ethyl)-methyl-amino]-1H-indazole-6-carboxylic acid 4-chloro-benzylamide,
3-[(2-Dimethylamino-ethyl)-methyl-amino]-1H-indazole-6-carboxylic acid 2,4-dichloro-benzylamide,
3-[(2-Dimethylamino-ethyl)-methyl-amino]-1H-indazole-6-carboxylic acid 4-trifluoromethyl-benzylamide,
3-[(2-Dimethylamino-ethyl)-methyl-amino]-1H-indazole-6-carboxylic acid 4-trifluoromethoxy-benzylamide,
3-[(2-Dimethylamino-ethyl)-methyl-amino]-1H-indazole-6-carboxylic acid (4-benzyloxy-phenyl)-amide,
3-(4-Methyl-piperazin-1-yl)-1H-indazole-5-carboxylic acid (4-benzyloxy-phenyl)-amide,
3-(4-Methyl-piperazin-1-yl)-1H-indazole-5-carboxylic acid biphenyl-4-ylamide,
3-(4-Methyl-piperazin-1-yl)-1H-indazole-5-carboxylic acid 4-chloro-benzylamide, and
1-Methyl-3-(4-methyl-piperazin-1-yl)-1H-indazole-5-carboxylic acid (4-benzyloxy-phenyl)-amide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in a free base form or solvate thereof, or in the form of a pharmaceutically acceptable salt or solvate thereof) can also be in the form of a polymorph, and

wherein if the compound exhibits chirality it can be in the form of a mixture of enantiomers such as a racemate, or a mixture of diastereomers, or can be in the form of a single enantiomer or a single diastereomer.

In another aspect, the present invention includes compounds of formula II:

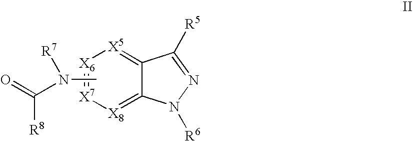

wherein
X⁵ to X⁸ are each, independently, N, CH, CR, or C—, wherein C— represents the point of attachment of group —NR⁷C(O)R⁸;
R is alkyl, alkenyl, alkynyl, cycloalkyl, alkylcycloalkyl, aryl, alkylaryl, heteroaryl, alkylheteroaryl, heterocycle, alkylheterocycle, halogen, hydroxyl, cyano, alkoxy, arylaoxy, cycloalkyloxy, alkoxycarbonyl, carboxyl, amino, alkylamino, dialkylamino, —SH, thioalkyl, alkylsulfonyl, alkylsulfinyl, arylsulfonyl, arylsulfinyl, aminosulfonyl, aminosulfinyl, acyl or aroyl;
R⁵ is heterocycle, heteroaryl, or NR^{c}R^{d} where
R^{c} and R^{d} are independently H, alkyl, alkenyl, alkynyl, aminoalkyl, alkylamido, cycloalkyl, alkylcycloalkyl, aryl, alkylaryl, heteroaryl, alkylheteroaryl, heterocycle, alkylheterocycle, alkylsulfonyl, or arylsulfonyl, with the proviso that R^{c} and R^{d} and not both H,
R⁶ is H, alkyl, alkenyl, alkynyl, cycloalkyl, alkylcycloalkyl, aryl, alkylaryl, heteroaryl, alkylheteroaryl, heterocycle, alkylheterocycle, aroyl or acyl;
R⁷ is H, alkyl, alkenyl, alkynyl, cycloalkyl, alkylcycloalkyl, aryl, alkylaryl, heteroaryl, alkylheteroaryl, heterocycle, or alkylheterocycle;
R⁸ is -D-E-F, where
D is alkylene, —NR¹⁰(aryl), —NR¹⁰(heteroaryl), alkylcycloalkyl, alkylaryl, or alkylheteoraryl;
E is —O—, —NR¹¹—, or —(CO)— and
F is alkylaryl, alkylheteroaryl or alkylheterocycle;
R⁹, R¹⁰ and R¹¹ are each, independently, H, alkyl, alkenyl, alkynyl, cycloalkyl, cycloalkylalkyl, aryl, alkylaryl heteroaryl, alkylheteroaryl, heterocycle, or alkylheterocycle;

and pharmaceutically acceptable salts or solvates (e.g., hydrates), or solvates of pharmaceutically acceptable salts thereof.

According to further embodiments of the compounds of Formula II, the —NR⁷C(O)R⁸ group is attached at the 4-position. In another embodiment, the —NR⁷C(O)R⁸ group is attached at the 5-position. In yet another embodiment, the —NR⁷C(O)R⁸ group is attached at the 6-position. In a further embodiment, the —NR⁷C(O)R⁸ group is attached at the 7-position. In yet a further embodiment, the —NR⁷C(O)R⁸ group is attached at the 5-position or the 6-position.

In one embodiment, X⁵, X⁶ and X⁸ are CH and X⁷ is —NR⁷C(O)R⁸. In another embodiment, X⁵, X⁷ and X⁸ are CH and X⁶ is —NR⁷C(O)R⁸. In a further embodiment, X⁵ and X⁸ are CH, one of X⁶ and X⁷ is CH and the other of X⁶ and X⁷ is —NR⁷C(O)R⁸.

In other embodiments, one of X⁵-X⁸ is N (e.g., X⁵ and X⁷ are CH or CR, X⁸ is N, and X⁶ is C—; X⁵ and X⁶ are CH or CR, X⁷ is C—, and X⁸ is N; X⁵ is N, X⁶ is C—, and X⁷ and X⁸ are CH or CR; X⁵ is N, X⁶ and X⁸ are CH or CR and X⁷ is C—). According to an additional embodiment, each of X⁵-X⁸ that is not substituted by the —NR⁷COR⁸ group is CH or CR. According to a further embodiment, each of X⁵—X⁸ that is not substituted by the —NR⁷COR⁸ group is CH.

In a further embodiment of the compounds of formula II, R⁵ is a 5 or 6-membered heterocycle (e.g., substituted or unsubstituted piperazinyl, homopiperazinyl, morpholinyl, pyrrolidinyl (e.g., 4-methyl-piperazin-1-yl, 4-methylsulfonyl-piperazin-1-yl, 4-methyl-homopiperazin-1-yl), a 5 or 6-membered heteroaryl (e.g., substituted or unsubstituted oxazolyl, imidazolyl; triazolyl, tetrazolyl, pyridinyl, pyrimidinyl), or NR^{c}R^{d}. For example R⁵ is substituted or unsubstituted piperidinyl, hompopiperidinyl, oxazolyl or imidazolyl, imidazolylmethyl, thiazolylmethyl or NR^{c}R^{d}.

In each case, when R⁵ is heterocycle, the heterocycle may optionally be bridged by a carbon chain linker (e.g., a 1 or 2 carbon atom linker) so as to form a bicyclic group

According to a further aspect of the invention, R^{c} and R^{d} are independently H, alkyl (e.g., methyl), aminoalkyl (e.g., dimethylaminoethyl, dimethylaminopropyl), amidoalkyl (e.g., CH₂CONH₂, CH₂CONHalkyl (e.g., CH₂CONHCH₃) CH₂CONH(alkyl)₂ (e.g., CH₂CON(CH₃)₂), alkylheteroaryl (e.g., thiazolylmethyl, oxazolylmethyl), alkylheterocycle (e.g., piperidinylethyl), alkylsulfonyl (e.g., methylsulfonyl), with the proviso that R^{c} and R^{d} and not both H. For example, one of R^{c} and R^{d} is hydrogen or alkyl (e.g., methyl) and the other of R^{c} and R^{d} is aminoalkyl (e.g., dimethylaminoethyl), amidoalkyl (e.g., CH₂CONH₂, CH₂CONHalkyl (e.g., CH₂CONHCH₃) CH₂CONH(alkyl)₂ (e.g., CH₂CON(CH₃)₂), alkylheteroaryl (e.g., thiazolylmethyl, oxazolylmethyl), alkylheterocycle (e.g., piperidinylethyl), or alkylsulfonyl (e.g., methylsulfonyl).

For further example, R⁵ is —NHCH₂CH₂NMe₂, —N(CH₃)CH₂CH₂NMe₂, —NHCH₂CH₂(piperidine), —NHCH₂CH₂CH₂NMe₂, —N(CH₃)CH₂C(O)NHMe, —N(CH₃)CH₂C(O)NMe₂, 4-methylpiperazinyl, 4-methylhomopiperazinyl, 4-methylsulfonylpiperazinyl, oxazolyl, imidazolyl, oxazolylmethyl or thiazolylmethyl.

In another embodiment R⁶ is H, alkyl, cycloalkyl, or alkylcycloalkyl. For example, R⁶ is H or alkyl (e.g., methyl). In certain embodiments, R⁶ is H.

In another embodiment R⁷ is H, alkyl, cycloalkyl, or alkylcycloalkyl. For example, R⁷ is H or alkyl (e.g., methyl). In certain embodiments, R⁷ is H.

According to additional embodiments, R⁹ is -D-E-F, where D is alkylene, —NR¹⁰(aryl), —NR¹⁰(heteroaryl), alkylcycloalkyl, alkylaryl, or alkylheteoraryl, E is —O— or —NR¹¹—, and F is alkylaryl, alkylheteroaryl or alkylheterocycle. In certain embodiments D is —NR¹⁰(aryl) or alkylaryl, E is —O— and F is alkylaryl. For example, R⁸ is alkylene-O-alkylaryl, alkylene-O-alkylheteroaryl, —NR¹⁰(aryl)-O-alkylaryl, —NR¹⁰(aryl)-O-alkylheteroaryl, alkylaryl-O-alkylaryl, alkylheteroaryl-O-alkylaryl, alkylaryl-O-alkylheteroaryl, or alkylheteroaryl-O-alkylheteroaryl. For example, R⁸ is —NR¹⁰(aryl)-Y-alkylaryl (such as —NR¹⁰(aryl)-O-alkylaryl, for example —N(H)—C₆H₄—O-benzyl) or alkylaryl-Y-alkylaryl (such as alkylaryl-O-alkylaryl, for example, -benzyl-O-benzyl.

In certain embodiments, when D and F contain an aryl group, said aryl group is phenyl or phenylene. For example, R⁸ is -benzyl-O-benzyl or —NH-phenylene-O-benzyl, e.g., —CH₂C₆H₄-p-O—CH₂C₆H₅ or —NH—C₆H₄-p-O—CH₂C₆H₅).

In additional embodiments, when D is —NR¹⁰(aryl), —NR¹⁰(heteroaryl), alkylaryl, or alkylheteoraryl, then E is a para-substituent on the aryl or heteroaryl ring. For example, R⁸ is 4-benzyloxybenzyl, or —NH—C₆H₄-p-O-benzyl.

According to a further embodiment, R⁹ is H, alkyl, alkenyl, alkynyl, cycloalkyl, alkylcycloalkyl, or aryl, e.g, R⁹ is H or alkyl (e.g., methyl). In one embodiment, R⁹ is H.

According to a further embodiment, R¹⁰ is H, alkyl, alkenyl, alkynyl, cycloalkyl, alkylcycloalkyl, or aryl, e.g, R¹⁰ is H or alkyl (e.g., methyl). In one embodiment, R¹⁰ is H.

According to a further embodiment, R¹¹ is H, alkyl, alkenyl, alkynyl, cycloalkyl, alkylcycloalkyl, or aryl, e.g, R¹¹ is H or alkyl (e.g., methyl). In one embodiment, R¹¹ is H.

In additional embodiments, the present invention includes compounds of formula II:

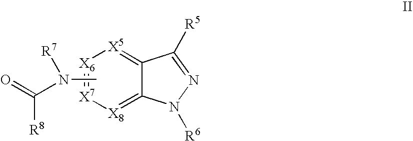

wherein
X⁵ and X⁸ are CH;
X⁶ to X⁷ are each, independently, CH or C—, wherein C— represents the point of attachment of group —NR⁷(CO)R⁸;
R⁵ is heterocycle, heteroaryl, or NR^{c}R^{d} where
R^{c} and R^{d} are independently H, alkyl, aminoalkyl, alkylamido, alkylcycloalkyl, alkylheteroaryl, alkylheterocycle or alkylsulfonyl, with the proviso that R^{c} and R^{d} and not both H, or
R⁶ is H or alkyl;
R⁷ is H;
R⁸ is -D-E-F, where
D is —NR¹⁰(aryl) or alkylaryl;
E is —O—;
F is alkylaryl; and
R¹⁰ is H;

and pharmaceutically acceptable salts or solvates (e.g., hydrates), or solvates of pharmaceutically acceptable salts thereof.

In another embodiment of the compounds of formula II, X⁵ and X⁸ are CH, R⁵ is NR^{c}R^{d}, one of X⁶ and X⁷ is CH and the other is —NR⁷(CO)R⁸ where R⁸ is D-E-F (in which D is alkylaryl, E is —O— and F is alkylaryl). For example, X⁵ and X⁸ are CH, R⁵ is NR^{c}R^{d} where R^{c} is H and R^{d} is aminoalkyl (e.g., —CH₂CH₂CH₂NMe₂) or alkylheterocycle (e.g., piperidinylethyl), one of X⁶ and X⁷ is CH and the other is —NR⁷(CO)R⁸ where R⁸ is D-E-F (in which D is benzyl, E is —O— and F is benzyl).

According to a compound and/or method aspect of the invention, the compound of formula II is chosen from:
2-(4-Benzyloxy-phenyl)-N-[3-(4-methyl-piperazin-1-yl)-1H-indazol-5-yl]-acetamide,
2-(4-Benzyloxy-phenyl)-N-{3-[(2-dimethylamino-ethyl)-methyl-amino]-1H-indazol-5-yl}-acetamide,
2-(4-Benzyloxy-phenyl)-N-[3-(4-methyl-piperazin-1-yl)-1H-indazol-6-yl]-acetamide,
2-(4-Benzyloxy-phenyl)-N-[3-(4-methyl-homopiperazin-1-yl)-1H-indazol-5-yl]-acetamide,
2-(4-Benzyloxy-phenyl)-N-[3-(2-Piperidin-1-yl-ethylamino)-1H-indazol-5-yl]-acetamide,
2-(4-Benzyloxy-phenyl)-N-[3-(3-dimethylaminopropylamino)-1H-indazol-5-yl]-acetamide,
{[3-(4-Methylpiperazinyl)(1H-indazol-6-yl]amino}-N-(4-phenoxyphenyl)carboxamide,
2-(4-Benzyloxy-phenyl)-N-(3-oxazol-2-yl-1H-indazol-5-yl)-acetamide,
2-(4-Benzyloxy-phenyl)-N-{3-[(thiazol-2-ylmethyl)-amino]-1H-indazol-5-yl}-acetamide
2-(4-Benzyloxy-phenyl)-N-(3-imidazol-1-yl-1H-indazol-5-yl)-acetamide,
2-(4-Benzyloxy-phenyl)-N-{3-[(oxazol-2-ylmethyl)-amino]-1H-indazol-5-yl}-acetamide,
2-(4-Benzyloxy-phenyl)-N-{3-[(4-Methylsulfonyl)-piperazin-1-yl]-1H-indazol-5-yl}-acetamide,
N-{3-[Methyl(methylsulfonylamino](1H-indazol-5-yl)}-2-(4-Benzyloxy-phenyl)acetamide,
N-(3-{[2-(Dimethylamino)ethyl]amino}(1H-indazol-5-yl)-2-(4-benzyloxy-phenyl)acetamide,
N,N-Dimethyl-2-[methyl(5-[2-(4-benzyloxy-phenyl)acetylamino](1H-indazol-3-yl))amino]acetamide, and
N-Methyl-2-[methyl(5-[2-(4-benzyloxy-phenyl)acetylamino](1H-indazol-3-yl))amino]acetamide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in a free base form or solvate thereof, or in the form of a pharmaceutically acceptable salt or solvate thereof) can also be in the form of a polymorph, and

wherein if the compound exhibits chirality it can be in the form of a mixture of enantiomers such as a racemate, or a mixture of diastereomers, or can be in the form of a single enantiomer or a single diastereomer.

In another aspect, the present invention relates to compounds of formula (III):

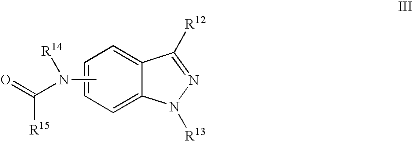

wherein

R¹² is NR^{e}R^{f} where R^{e} and R^{f} are each, independently, H, alkyl (e.g., methyl), alkylheterocycle or alkylheteraryl, with the proviso that R^{e} and R^{f} are not both H;

R¹³ and R⁴ are each independently H or alkyl (e.g., methyl); and

R¹⁵ is heteroaryl other than benzimidazolyl;

and pharmaceutically acceptable salts or solvates (e.g., hydrates), or solvates of pharmaceutically acceptable salts thereof.

In one embodiment, R^{e} and R^{f} are each, independently, H, alkylheterocycle (e.g., piperidinylethyl) or alkylheteroaryl (e.g., substituted or unsubstituted thiazolylmethyl, such as 5-methyl-thiazolylmethyl).

In additional embodiments, R¹³ is H. In another embodiment R¹⁴ is H.

In further embodiments, R¹⁵ is a substituted or unsubstituted 5-membered heteroaryl (e.g., substituted or unsubstituted thienyl, furanyl, imidazolyl). In one embodiment, R¹⁵ is substituted or unsubstituted thienyl (e.g., phenyl substituted thienyl, such as, 5-(4-chlorophenyl)-2-thienyl).

According to a compound and/or method aspect of the invention, the compound of formula III is chosen from:
5-(4-Chloro-phenyl)-thiophene-2-carboxylic acid [3-(2-piperidin-1-ylethylamino)-3a,7a-dihydro-1H-indazol-6-yl]-amide, and
5-(4-Chloro-phenyl)-thiophene-2-carboxylic acid {3-[(5-methyl-thiazol-4-ylmethyl)-amino]-1H-indazol-6-yl}-amide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in a free base form or solvate thereof, or in the form of a pharmaceutically acceptable salt or solvate thereof) can also be in the form of a polymorph, and

wherein if the compound exhibits chirality it can be in the form of a mixture of enantiomers such as a racemate, or a mixture of diastereomers, or can be in the form of a single enantiomer or a single diastereomer.

As used herein the term “halogen” means F, Cl, Br, and I.

The term “alkyl” means a substituted or unsubstituted saturated hydrocarbon radical which may be straight-chain or branched-chain and contains about 1 to about 20 carbon atoms, for instance 1 to 12 carbon atoms, such as 1 to 8 carbon atoms, e.g., 1 to 4 carbon atoms. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, methylhexyl, dimethylpentyl, ethylpentyl, ethylmethylbutyl, dimethylbutyl, and the like.

Substituted alkyl groups are alkyl groups as described above which are substituted in one or more positions by, e.g., halogen, hydroxyl, amino, carboxy, and cyano, and combinations thereof.

The term “alkenyl” means a substituted or unsubstituted hydrocarbon radical which may be straight-chain or branched-chain, which contains one or more carbon-carbon double bonds, and which may comprise about 1 to about 20 carbon atoms, such as 1 to 12 carbon atoms, for instance 1 to 6 carbon atoms. Suitable alkenyl groups include ethenyl, propenyl, butenyl, etc.

Substituted alkenyl groups are alkenyl groups as described above which are substituted in one or more positions by, e.g., halogen, hydroxyl, amino, carboxy, cyano, and combinations thereof.

The term “alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

The term “alkynyl” means a substituted or unsubstituted aliphatic hydrocarbon radical which may be straight-chain or branched-chain and which contains one or more carbon-carbon triple bonds. Preferably the alkynyl group contains 2 to 15 carbon atoms, such as 2 to 12 carbon atoms, e.g., 2 to 8 carbon atoms. Suitable alkynyl groups include ethynyl, propynyl, butynyl, etc.

Substituted alkynyl groups are alkynyl groups as described above which are substituted in one or more positions by, e.g., halogen, hydroxyl, amino, carboxy, cyano, and combinations thereof.

The term “alkylcycloalkyl” means a cycloalkyl-alkyl-group, where cycloalkyl and alkyl are as described above.

The term “amino” means —NH₂.

The term “alkylamino” means —NH(alkyl), wherein alkyl is as described above.

The term “dialkylamino” means —N(alkyl)₂, wherein alkyl is as described above.

The term “alkylsulfonyl” means an —SO₂-alkyl group, wherein alkyl is as described above.

The term “alkylsulfinyl” means an —SO-alkyl group, wherein alkyl is as described above.

The term “aryl” means a substituted or unsubstituted aromatic monocyclic or bicyclic ring system comprising about 5 to about 14 carbon atoms, preferably about 6 to about 10 carbon atoms. Suitable aryl groups include, but are not limited to, phenyl, naphthyl, tetrahydronaphthyl, indanyl and indenyl.

Substituted aryl groups include the above-described aryl groups which are substituted one or more times by, for example, alkyl, alkenyl, alkynyl, cycloalkyl, alkylcycloalkyl, aryl, alkylaryl, heteroaryl, alkylheteroaryl, heterocycle, alkylheterocycle, halogen, hydroxyl, cyano, alkoxy, arylaoxy, cycloalkyloxy, alkoxycarbonyl, carboxyl, amino, alkylamino, dialkylamino, —SH, thioalkyl, alkylsulfonyl, alkylsulfinyl, arylsulfonyl, arylsulfinyl, aminosulfonyl, aminosulfinyl, aroyl, acyl, and combinations thereof.

The term “arylsulfonyl” means an —SO₂-aryl group, wherein aryl is as described above.

The term “arylsulfinyl” means an —SO-aryl group, wherein aryl is as described above.

The term “carboxyl” means —C(O)OH.

The term “cycloalkyl” means a monocyclic, bicyclic or tricyclic nonaromatic saturated hydrocarbon radical having 3 to 10 carbon atoms, such as 3 to 8 carbon atoms, for example, 3 to 6 carbon atoms. Suitable cycloalkyl groups include, but are not limited to, cyclopropyl, cyclobutyl, cyclopentyl, cyclohexyl, cycloheptyl, cyclooctyl, norbornyl, 1-decalin, adamant-1-yl, and adamant-2-yl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, bicyclo[4.2.0]octyl, and spiro[3.5]nonyl. Preferred cycloalkyl groups include cyclopropyl, cyclobutyl, cyclopentyl and cyclohexyl. The cycloalkyl group can be substituted, for example, by one or more halogens and/or alkyl groups.

The term “heteroaryl” means a substituted or unsubstituted aromatic monocyclic or multicyclic ring system comprising 5 to about 10 ring atoms, preferably 5 or 6 ring atoms, wherein at least one of the ring atoms is an N, O or S atom. Suitable heteroaryl groups include, but are not limited to furyl, thienyl, pyrrolyl, pyrazolyl, imidazolyl, pyridyl, pyrimidinyl, indolyl, quinolinyl, isoquinolinyl, naphthyridinyl and the like.

Substituted heteroaryl groups include the above-described heteroaryl groups which are substituted one or more times by, for example, alkyl, alkenyl, alkynyl, cycloalkyl, alkylcycloalkyl, aryl, alkylaryl, heteroaryl, alkylheteroaryl, heterocycle, alkylheterocycle, halogen, hydroxyl, cyano, alkoxy, arylaoxy, cycloalkyloxy, alkoxycarbonyl, carboxyl, amino, alkylamino, dialkylamino, —SH, thioalkyl, alkylsulfonyl, alkylsulfinyl, arylsulfonyl, arylsulfinyl, aminosulfonyl, aminosulfinyl, aroyl, acyl, and combinations thereof.

The term “heterocycle” means a substituted or unsubstituted non-aromatic mono- or multicyclic ring system comprising 3 to 10 atoms, preferably 5 or 6 atoms, wherein at least one of the ring atoms is an N, O or S atom. Suitable heterocyle groups include, but are not limited to tetrahydrofuranyl, tetrahydrothienyl, tetrahydropyranyl, dihydropyranyl, pyrrolidinyl, piperidinyl, piperazinyl, morpholinyl, isoxazolinyl, and the like

Substituted heterocycle groups include the above-described heterocycle groups which are substituted one or more times by, for example, alkyl, alkenyl, alkynyl, cycloalkyl, alkylcycloalkyl, aryl, alkylaryl, heteroaryl, alkylheteroaryl, heterocycle, alkylheterocycle, halogen, hydroxyl, cyano, alkoxy, arylaoxy, cycloalkyloxy, alkoxycarbonyl, carboxyl, amino, alkylamino, dialkylamino, —SH, thioalkyl, alkylsulfonyl, alkylsulfinyl, arylsulfonyl, arylsulfinyl, aminosulfonyl, aminosulfinyl, aroyl, acyl, and combinations thereof.

The term “aroyl” means an aryl-C(O)—, in which the aryl group is as previously described. Suitable aroyl groups include, but are not limited to, benzoyl and 1-naphthoyl.

The term “acyl” means an HC(O)—, alkyl-C(O)—, or cycloalkyl-C(O)—, in which the alkyl and cycloalkyl groups are as previously described.

The term “alkoxy” means alkyl-O— groups and in which the alkyl portion is in accordance with the previous discussion. Suitable alkoxy groups include, but are not limited to, methoxy, ethoxy, n-propoxy, iso-propoxy, n-butoxy, t-butoxy, pentoxy, hexoxy, heptoxy, octoxy, and the like. For example, the alkoxy can be methoxy or ethoxy.

The term “alkylaryl” refers to an aryl-alkyl-radical in which the aryl and alkyl portions are in accordance with the previous descriptions. Suitable examples include, but are not limited to, benzyl, 1-phenethyl, 2-phenethyl, phenpropyl, phenbutyl, phenpentyl, and napthylmethyl

The term “alkylheterocycle” refers to a heterocycle-alkyl-group wherein the heterocycle and alkyl portions are in accordance with the previous discussions.

The term “alkylheteroaryl” refers to a heteroaryl-alkyl-group wherein the heteroaryl and alkyl portions are in accordance with the previous discussions. Suitable examples include, but are not limited to, pyridylmethyl, thiazolylmethyl, thienylmethyl, pyrimidinylmethyl, pyrazinylmethyl, and isoquinolinylmethyl

The term “aryloxy” means an aryl-O— group, in which the aryl group is as previously described.

The term “alkylaryloxy” means aryl-alkyl-O—, in which the aryl and alkyl groups are as previously described.

The term “alkylthio” means an alkyl-S— group, in which the alkyl group is as previously described.

The term “arylthio” means an aryl-S— group, in which the aryl group is as previously described.

The term “alkoxycarbonyl” means an alkyl-O—CO— group, in which the alkyl group is as previously described.

The term “aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl, and R′ is selected from hydrogen, alkyl, hydroxyalkyl, alkoxyalkyl, aryl, aralkyl, heteroaryl, heteroaralkyl, or haloalkyl, e.g, aminomethyl, methylaminoethyl, 2-ethylamino-2-methylethyl, 1,3-diaminopropyl, dimethylaminoethyl, dimethylaminomethyl, diethylaminoethyl, acetylaminopropyl, and the like.

The term “amidoalkyl” means a linear monovalent hydrocarbon radical of one to six carbon atoms or a branched monovalent hydrocarbon radical of three to six carbons substituted with at least one, preferably one or two, —(CO)NRR′ where R is hydrogen, alkyl, or —COR^{a} where R^{a} is alkyl, and R′ is selected from hydrogen, alkyl, hydroxyalkyl, alkoxyalkyl, aryl, aralkyl, heteroaryl, heteroaralkyl, or haloalkyl, e.g, CH₂CONH₂, CH₂CONHalkyl (e.g., CH₂CONHCH₃), CH₂CONH(alkyl)₂ (e.g., CH₂CON(CH₃)₂), and the like.

The term aminosulfinyl” means a —SONRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl as defined above, e.g., —SONH₂, methylaminosulfinyl, 2-dimethylaminosulfinyl, and the like.

The term “aminosulfonyl” means a —SO₂NRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl as defined above, e.g., —SO₂NH₂, methylaminosulfonyl, 2-dimethylaminosulfonyl, and the like.

The term “aryloxycarbonyl” means an aryl-O—C(O)— group, in which the aryl group is as previously described.

One of ordinary skill in the art will recognize that compounds of Formulas I-III can exist in different tautomeric and geometrical isomeric forms. All of these compounds, including cis isomers, trans isomers, diastereomic mixtures, racemates, nonracemic mixtures of enantiomers, substantially pure, and pure enantiomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of formulas I-III can likewise be obtained by utilizing optically active starting materials in chiral synthesis processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] (2000), 110 pp. CAN 133:68895 AN 2000:473538 CAPLUS; Kabalka, George W.; Varma, Rajender S. The synthesis of radiolabeled compounds via organometallic intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64(1-2), 9-32. CODEN: JRACBN ISSN:0022-4081, CAN 95:76229 AN 1981:476229 CAPLUS.

Where applicable, the present invention also relates to useful forms of the compounds as disclosed herein, such as base free forms, and pharmaceutically acceptable salts or prodrugs of all the compounds of the present invention for which salts or prodrugs can be prepared. Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, a hydrobromide, a hydroformate, or a maleate.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of formulas I-III can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of formulas I-III can exist in different solvate forms. Solvates of the compounds of the invention may also form when solvent molecules are incorporated into the crystalline lattice structure of the compound molecule during the crystallization process.

The term “prodrug” means a compound that is a drug precursor which upon administration to a subject undergoes chemical conversion by metabolic or chemical processes to yield a compound of the present invention. Such prodrugs are considered to be within the scope of this invention.

The compounds of the invention can be administered alone or as an active ingredient of a formulation. Thus, the present invention also includes pharmaceutical compositions of compounds of formulas I-III, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration of the compounds of the present invention may be accomplished according to patient needs, for example, orally, nasally, parenterally (subcutaneously, intraveneously, intramuscularly, intrasternally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

The compounds of the present invention may be useful as MCH receptor ligands. For example, the compounds of the present invention may be useful as MCHI-1 ligands. Thus, the compounds of the present invention may be useful in the treatment of conditions that respond to MCH receptor ligands, e.g., MCH antagonists, such as MCH-1 antagonists.

According to additional embodiments, the present invention provides methods for treating a condition that responds to a MCH ligand, e.g., an MCH antagonist, such as an MCH-1 antagonist, which involve administering to a patient in need thereof an effective amount of a compound of the present invention.

In certain embodiments, the compounds of the present invention may be useful in the treatment of MCH related conditions including central nervous system disorders, cardiovascular system disorders, gastrointestinal system disorders, eating disorders, obesity, sleeping disorders, childhood disorders, cognitive disorders, mental disorders, substance related disorders, psychotic disorders, mood disorders, depression, sexual disorders and neurodegenerative diseases.

For example, the compounds of the present invention may be useful in the treatment of obesity, bulimia, bulimia nervosa, major depressive disorder, bipolar I and II disorders, schizoaffective disorder, cognitive disorders with depressed mood, anxiety, epilepsy, delirium, dementia, schizophrenia, attention-deficit hyperactivity disorder, memory impairment, personality disorders, insomnia, hypersomnia, narcolepsy, circadian rhythm sleep disorder, nightmare disorder, sleep terror disorder, sleepwalking disorder, obsessive-compulsive disorder, panic disorder, with or without agoraphobia, posttraumatic stress disorder, social anxiety disorder, social phobia, generalized anxiety disorder, diabetes, hormone disorder, hyperlipidemia, gout, fatty liver, hepatitis cirrhosis, stenocardia, acute or congestive heart failure, myocardial infarction, coronary atherosclerosis, hypertension, renal diseases. electrolyte abnormality, emotional disturbance, cognitive failure, dyskinesia, paresthesias, smell disorders, morphine tolerance, drug dependence, alcoholism, infertility, preterm labor, sexual dysfunction, abnormalities in reproduction and sexual behaviour, diuresis and water/electrolyte homeostatsis, thyroid hormone secretion, respiratory disorders, abnormalities in reproduction and sexual behavior and cancer.

In one embodiment, the compounds of the present invention may be useful in the treatment of eating disorders, weight gain, obesity, depression or anxiety. For example, the compounds of the present invention may be useful in the treatment of eating disorders, weight gain or obesity. In one embodiment, the compounds of the present invention may be useful in the treatment of obesity.

The term “treating” means to relieve, alleviate, delay, reduce, reverse, improve or prevent at least one symptom of a condition in a subject. The term “treating” may also mean to arrest, delay the onset (i.e., the period prior to clinical manifestation of a disease) and/or reduce the risk of developing or worsening a condition.

An “effective amount” means the amount of a compound of formulas I-III that, when administered to a patient (e.g., a mammal) for treating a disease, is sufficient to effect such treatment for the disease, or an amount of a compound of formulas I-III that is sufficient for antagonizing the MCH receptor (such as MCH-1) to achieve the objectives of the invention. The “effective amount” will vary depending on the compound, the disease and its severity and the age, weight, etc., of the patient to be treated.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

In some embodiments, the compounds of the present invention are administered as a mono-therapy. In other embodiments, the compounds of the present invention are administered as part of a combination therapy. For example, a compound of formulas I-III may be used in combination with other drugs or therapies that are used in the treatment/prevention/suppression or amelioration of the diseases or conditions for which compounds of formulas I-III are useful.

Such other drug(s) may be administered, by a route and in an amount commonly used therefor, contemporaneously or sequentially with a compound of formulas I-III. When a compound of formulas I-III is used contemporaneously with one or more other drugs, a pharmaceutical unit dosage form containing such other drugs in addition to the compound of formulas I-III may be employed. Accordingly, the pharmaceutical compositions of the present invention include those that also contain one or more other active ingredients, in addition to a compound of formulas I-III.

Examples of other active ingredients that may be combined with a compound of formulas I-III and either administered separately or in the same pharmaceutical compositions, include, but are not limited to:

(a) insulin sensitizers including (i) PPARγ agonists such as the glitazones (e.g. ciglitazone, troglitazone, pioglitazone, rosiglitazone, englitazone, isaglitazone (MCC-555), BRL49653 and the like), and compounds disclosed in WO 97/27857, WO 97/28115, WO 97/28137 and WO 97/27847; (ii) biguanides such as metformin and phenformin;

(b) insulin or insulin mimetics;

(c) sulfonylureas such as tolbutamide and glipizide;

(d) alpha-glucosidase inhibitors (such as acarbose);

(e) cholesterol lowering agents such as i. HMG-CoA reductase inhibitors (lovastatin, simvastatin and pravastatin, fluvastatin, atorvastatin, and other statins), ii. sequestrants (cholestyramine, colestipol and a dialkylaminoalkyl derivatives of a cross-linked dextran), iii. nicotinyl alcohol nicotinic acid or a salt thereof, iv. proliferator-activator receptor a agonists such as fenofibric acid derivatives (gemfibrozil, clofibrat, fenofibrate and benzafibrate), v. inhibitors of cholesterol absorption for example beta-sitosterol and (acyl CoA:cholesterol acyltransferase) inhibitors for example melinamide, vi. probucol, vii. vitamin E, and viii. thyromimetics;

(f) PPARδ agonists such as those disclosed in WO 97/28149;

(g) antiobesity compounds such as fenfluramine, dexfenfluramrine, phentermine, sibutramine, orlistat, and other 3 adrenergic receptor agonists;

(h) feeding behavior modifying agents such as neuropeptide Y antagonists (e.g. neuropeptide Y5) such as those disclosed in WO 97/19682, WO 97/20820, WO 97/20821, WO 97/20822 and WO 97/20823;

(i) PPARα agonists such as described in WO 97/36579 and beclofibrate, benzafibrate, ciprofibrate, clofibrate, etofibrate, and fenofibrate;

(j) PPARγ antagonists as described in WO 97/10813; and

(k) serotonin reuptake inhibitors such as fluoxetine, paroxetine and sertraline.

Additional active agents and therapies that may be used in combination with the compounds of the present invention are described in the section of International Publication No.: WO 2006/020277 set forth at page 27, line 28 to page 34, line 3, which is hereby incorporated by reference.

The present invention also provides processes for preparing the compounds of the present invention. For example, the compounds of the present invention may be prepared using the general reaction schemes outlined below. Starting materials (e.g., 3-amino-4-methyl benzoic acid methylester, 5-nitroindazole and 6-nitroindazole) are commercially available from Sigma-Aldrich (St. Louis, Mo.).

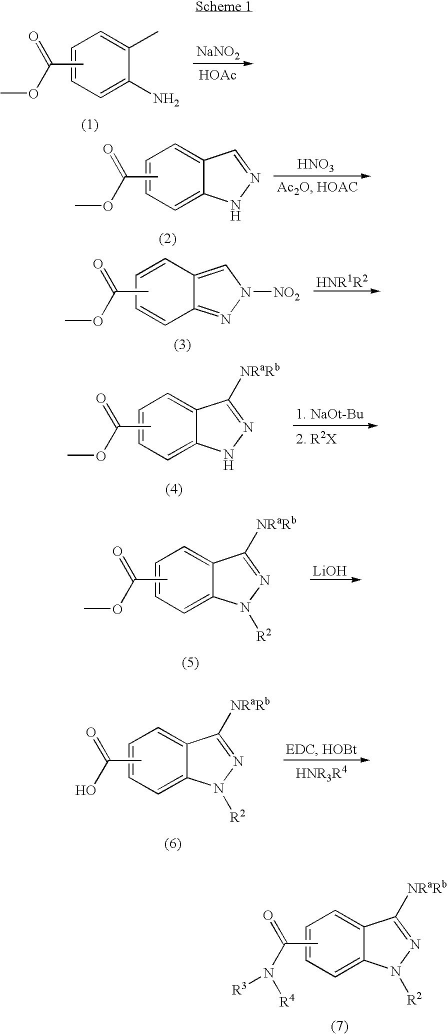

Diazotization of an amino-methyl-benzoic acid methyl ester (1) with sodium nitrite in acetic acid affords the desired carboxy indazole (2). Selective nitration with fuming nitric acid and acetic anhydride to give the nitro compound (3), followed by reaction with an appropriate amine provides the aminoindazole (4) in acceptable yields. N-alkylation of the indazole to give (5) is achieved by deprotonation with sodium tert-butoxide and alkylation with the desired alkyl halide. Ester hydrolysis followed by amide coupling of the resulting acid (6) under standard conditions provides the compounds of formula I.

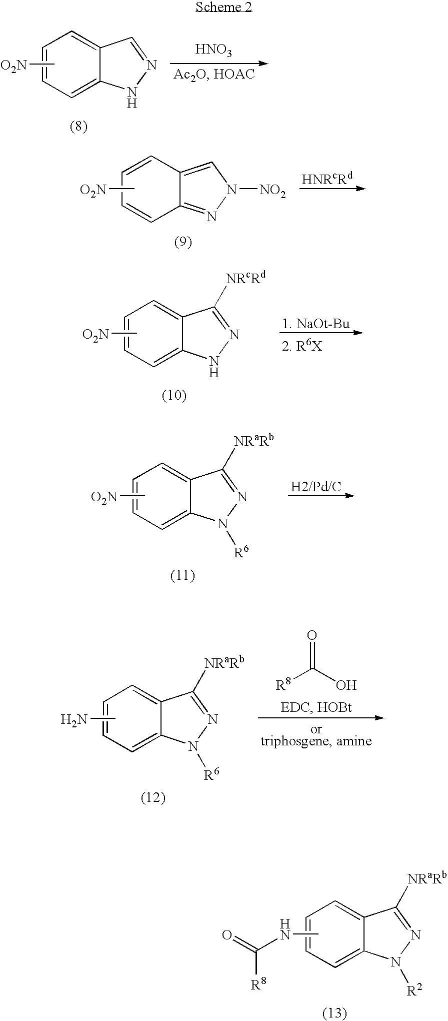

Selective nitration of an appropriate nitroindazole (8) with fuming nitric acid and acetic anhydride followed by reaction with an appropriate amine provides the aminoindazole (10) in acceptable yields. N-alkylation of the indazole to give (11) is achieved by deprotonation with sodium tert-butoxide and alkylation with the desired alkyl halide. Reduction of the nitro functionality to give the amine (12), followed by amide coupling under standard conditions provides the compounds of formula II.

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

In the foregoing and in the following examples, all temperatures are set forth uncorrected in degrees Celsius; and, unless otherwise indicated, all parts and percentages are by weight.

The entire disclosures of all applications, patents and publications, cited above and below, are hereby incorporated by reference.

### EXAMPLE 1

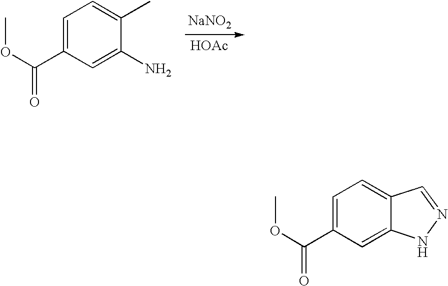

### 1H-Indazole-6-carboxylic acid methyl ester

To a mixture of 3-amino-4-methyl-benzoic acid methyl ester (15 g, 90.9 mmol) in HOAc (400 mL) was added a solution of NaNO₂ (6.27 g, 90.9 mmol) in H₂O (90 mL) dropwise. The mixture was stirred at ambient temperature for five hr. then concentrated in vacuo. The resulting solid was triturated with ethyl acetate (400 mL) and filtered. The filtrate was concentrated to afford the title compound (13.3 g, 83%). m/z (M+H)=177.18.

### EXAMPLE 2

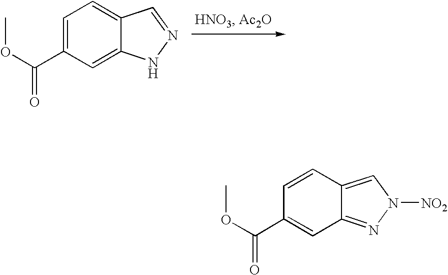

### 2-Nitro-2H-indazole-6-carboxylic acid methyl ester

A suspension of 1H-Indazole-6-carboxylic acid methyl ester (2 g, 11.4 mmol) in acetic acid (15 mL) was poured into a solution of HNO₃ (3 mL) and Ac₂O (7.5 mL) that was cooled to −5° C. After 2 min. the mixture was poured onto ice and stirred for 30 min. The precipitate was filtered and air dried. The material was used without further purification.

### EXAMPLE 3

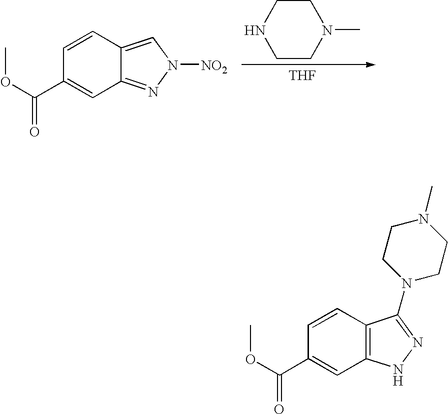

### 3-(4-Methyl-piperazin-1-yl)-1H-indazole-6-carboxylic acid methyl ester

To a solution of 2-Nitro-2H-indazole-6-carboxylic acid methyl ester (400 mg, 1.81 mmol) in THF (15 mL) was added 1-methylpiperazine (0.40 mL, 3.62 mmol). The mixture was stirred for 15 hr. then concentrated in vacuo. The residue was diluted with ethyl acetate (50 mL) and washed with sat. aq. sodium bicarbonate (25 mL). The aqueous layer was extracted with ethyl acetate (25 mL). The combined organic layers were dried over anhydrous sodium sulfate and concentrated in vacuo. Chromatography on silica yielded the title compound (242 mg, 49%). m/z (M+H)=275.33.

### EXAMPLE 4

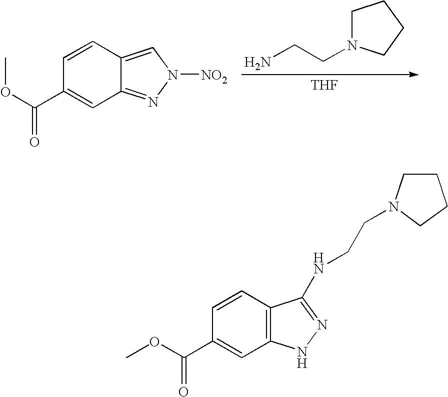

### 3-(2-Pyrrolidin-1-yl-ethylamino)-1H-indazole-6-carboxylic acid methyl ester

To a solution of 2-Nitro-2H-indazole-6-carboxylic acid methyl ester (400 mg, 1.81 mmol) in THF (15 mL) was added 2-Pyrrolidin-1-yl-ethylamine (0.46 mL, 3.62 mmol). The mixture was stirred for 15 h then concentrated in vacuo. The residue was diluted with ethyl acetate (50 mL) and washed with sat. aq. sodium bicarbonate (25 mL). The aqueous layer was extracted with ethyl acetate (25 mL). The combined organic layers were dried over anhydrous sodium sulfate and concentrated in vacuo. Chromatography on silica yielded the title compound (189 mg, 36%). m/z (M+H)=289.35.

### EXAMPLE 5

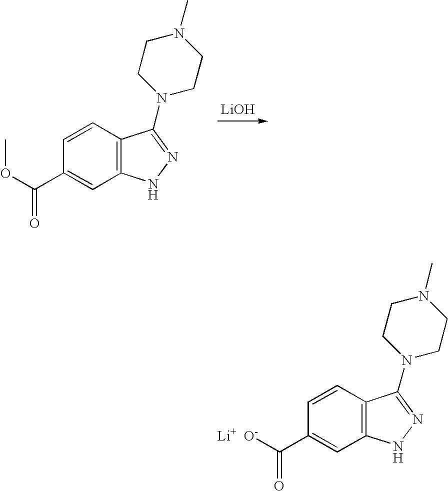

### Lithium; 3-(4-methyl-piperazin-1-yl)-1H-indazole-6-carboxylate

To a solution of 3-(4-Methyl-piperazin-1-yl)-1H-indazole-6-carboxylic acid methyl ester (821 mg, 3.00 mmol) in THF (10 mL), MeOH (10 mL) and water (5 mL) was added LiOH.H₂O (252 mg, 6.00 mmol). The mixture was heated at reflux for 2 hr. then allowed to cool. The mixture was concentrated in vacuo and used without further purification.

### EXAMPLE 6

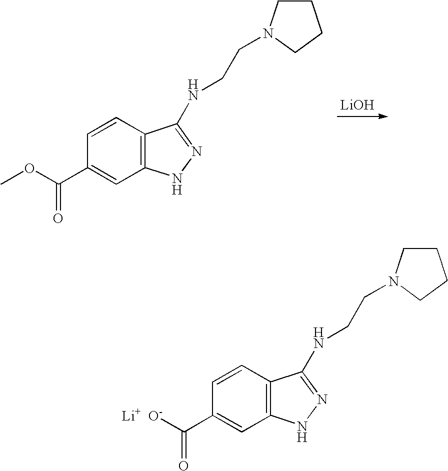

### Lithium; 3-(2-pyrrolidin-1-yl-ethylamino)-1H-indazole-6-carboxylate

To a solution of 3-(2-Pyrrolidin-1-yl-ethylamino)-1H-indazole-6-carboxylic acid methyl ester (900 mg, 3.13 mmol) in THF (10 mL), MeOH (10 mL) and water (5 mL) was added LiOH.H₂O (263 mg, 6.25 mmol). The mixture was heated at reflux for 2 hr. then allowed to cool. The mixture was concentrated in vacuo and used without further purification.

### EXAMPLE 7

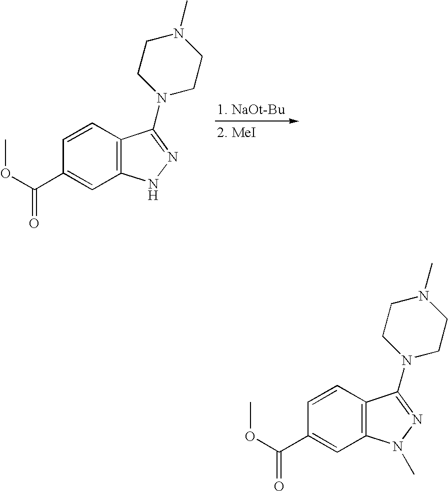

### 1-Methyl-3-(4-methyl-piperazin-1-yl)-1H-indazole-6-carboxylic acid methyl ester

A solution of 3-(4-Methyl-piperazin-1-yl)-1H-indazole-6-carboxylic acid methyl ester (777 mg, 2.84 mmol) in anhydrous DMF (15 mL) under a nitrogen atmosphere was cooled in an ice bath. Sodium tert-butoxide (300 mg, 3.12 mmol) was added and the mixture was stirred for 10 min. Methyl iodide (0.19 mL, 3.12 mmol) was added and the mixture was stirred for 30 min. The solution was diluted with ethyl acetate (50 mL) and washed with water (50 mL). The aqueous layer was extracted with ethyl acetate (50 mL). The combined organic layers were dried over anhydrous sodium sulfate and concentrated in vacuo. Chromatography on silica yielded the title compound (742 mg, 91%). m/z (M+H)=289.35.

### EXAMPLE 8

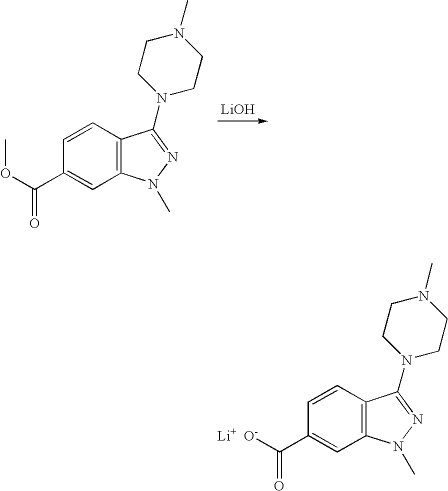

### Lithium; 1-methyl-3-(4-methyl-piperazin-1-yl)-1H-indazole-6-carboxylate

To a solution of 1-methyl-3-(4-methyl-piperazin-1-yl)-1H-indazole-6-carboxylic acid methyl ester (821 mg, 2.84 mmol) in THF (10 mL), MeOH (10 mL) and water (5 mL) was added LiOH.H₂O (239 mg, 5.68 mmol). The mixture was heated at reflux for 2 hr. then allowed to cool. The mixture was concentrated in vacuo and used without further purification.

### EXAMPLE 9

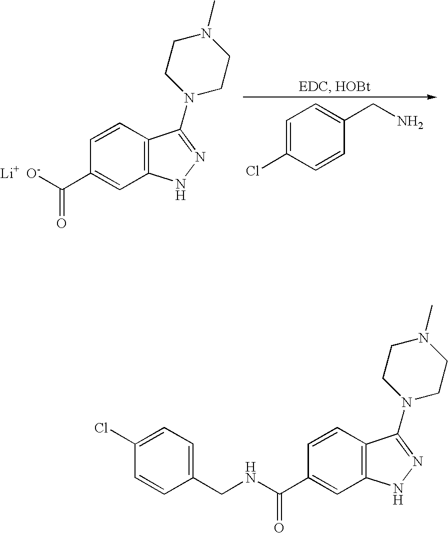

### 3-(4-Methyl-piperazin-1-yl)-1H-indazole-6-carboxylic acid 4-chloro-benzylamide

To a solution of lithium 3-(4-methyl-piperazin-1-yl)-1H-indazole-6-carboxylate (85 mg, 0.32 mmol) in anhydrous DMF (5 mL) was added EDC (N-(3-dimethylaminopropyl)-N′-ethylcarbodiimide hydrochloride) (73 mg, 0.38 mmol) and HOBt (1-hydroxybenzotriazole) (51 mg, 0.38 mmol). The mixture was stirred for 10 min. then Et₃N (0.11 mL, 0.80 mmol) and 4-chlorobenzylamine (46 μL, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (32 mg, 26%). m/z (M+H)=384.88.

### EXAMPLE 10

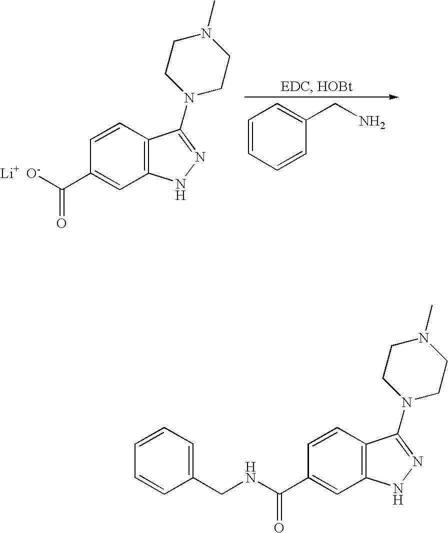

### 3-(4-Methyl-piperazin-1-yl)-1H-indazole-6-carboxylic acid benzylamide

To a solution of lithium 3-(4-methyl-piperazin-1-yl)-1H-indazole-6-carboxylate (93 mg, 0.35 mmol) in anhydrous DMF (5 mL) was added EDC (81 mg, 0.42 mmol) and HOBt (57 mg, 0.42 mmol). The mixture was stirred for 10 min. then Et₃N (0.24 mL, 1.75 mmol) and benzylamine (35 μL, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (34 mg, 34%). m/z (M+H)=350.44.

### EXAMPLE 11

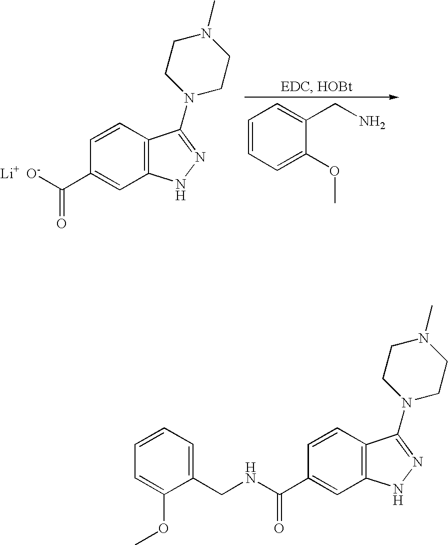

### 3-(4-Methyl-piperazin-1-yl)-1H-indazole-6-carboxylic acid 2-methoxy-benzylamide

To a solution of lithium 3-(4-methyl-piperazin-1-yl)-1H-indazole-6-carboxylate (93 mg, 0.35 mmol) in anhydrous DMF (5 mL) was added EDC (81 mg, 0.42 mmol) and HOBt (57 mg, 0.42 mmol). The mixture was stirred for 10 min. then Et₃N (0.24 mL, 1.75 mmol) and 2-methyoxybenzylamine (43 μL, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (29 mg, 26%). m/z (M+H)=380.47.

### EXAMPLE 12

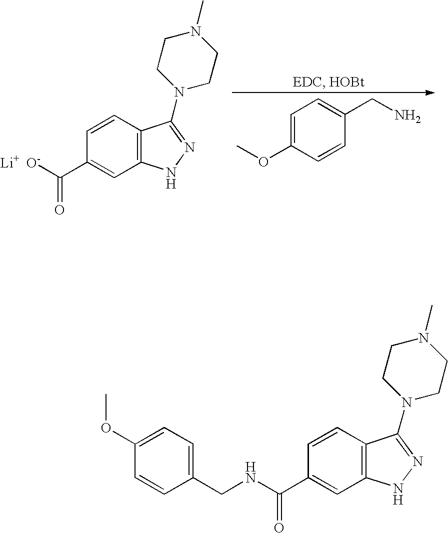

### 3-(4-Methyl-piperazin-1-yl)-1H-indazole-6-carboxylic acid 4-methoxy-benzylamide

To a solution of lithium 3-(4-methyl-piperazin-1-yl)-1H-indazole-6-carboxylate (93 mg, 0.35 mmol) in anhydrous DMF (5 mL) was added EDC (81 mg, 0.42 mmol) and HOBt (57 mg, 0.42 mmol). The mixture was stirred for 10 min. then Et₃N (0.24 mL, 1.75 mmol) and 4-methyoxybenzylamine (43 μL, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (31 mg, 28%). m/z (M+H)=380.47.

### EXAMPLE 13

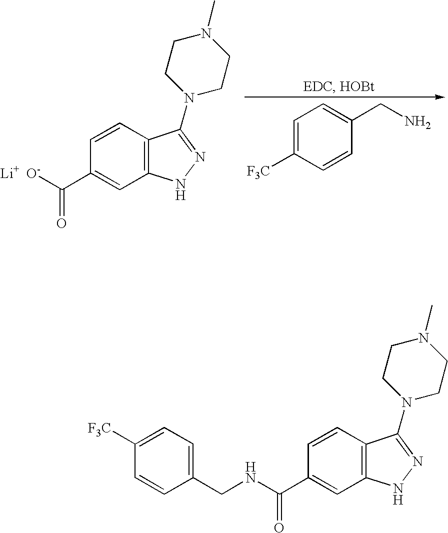

### 3-(4-Methyl-piperazin-1-yl)-1H-indazole-6-carboxylic acid 4-trifluoromethyl-benzylamide

To a solution of lithium 3-(4-methyl-piperazin-1-yl)-1H-indazole-6-carboxylate (93 mg, 0.35 mmol) in anhydrous DMF (5 mL) was added EDC (81 mg, 0.42 mmol) and HOBt (57 mg, 0.42 mmol). The mixture was stirred for 10 min. then Et₃N (0.24 mL, 1.75 mmol) and 4-trifluoromethylbenzylamine (47 μL, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (35 mg, 28%). m/z (M+H)=366.44.

### EXAMPLE 14

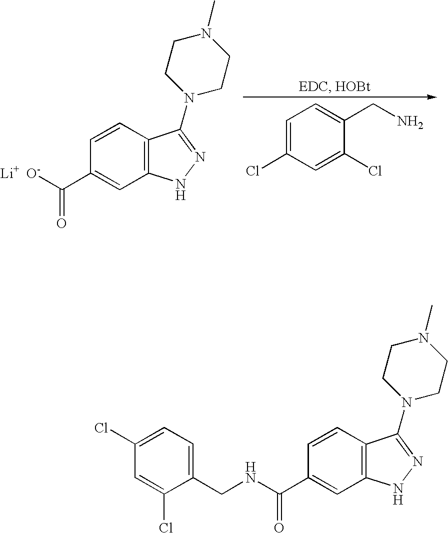

### 3-(4-Methyl-piperazin-1-yl)-1H-indazole-6-carboxylic acid 2,4-dichloro-benzylamide

To a solution of lithium 3-(4-methyl-piperazin-1-yl)-1H-indazole-6-carboxylate (93 mg, 0.35 mmol) in anhydrous DMF (5 mL) was added EDC (81 mg, 0.42 mmol) and HOBt (57 mg, 0.42 mmol). The mixture was stirred for 10 min. then Et₃N (0.24 mL, 1.75 mmol) and 2,4-dichlorobenzylamine (44 μL, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (29 mg, 23%). m/z (M+H)=419.33.

### EXAMPLE 15

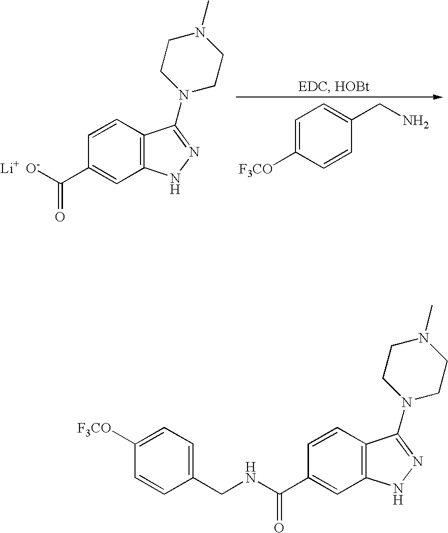

### 3-(4-Methyl-piperazin-1-yl)-1H-indazole-6-carboxylic acid 4-trifluoromethoxy-benzylamide

To a solution of lithium 3-(4-methyl-piperazin-1-yl)-1H-indazole-6-carboxylate (93 mg, 0.35 mmol) in anhydrous DMF (5 mL) was added EDC (81 mg, 0.42 mmol) and HOBt (57 mg, 0.42 mmol). The mixture was stirred for 10 min. then Et₃N (0.24 mL, 1.75 mmol) and 4-trifluoromethoxybenzylamine (50 μL, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (32 mg, 25%). m/z (M+H)=384.89.

### EXAMPLE 16

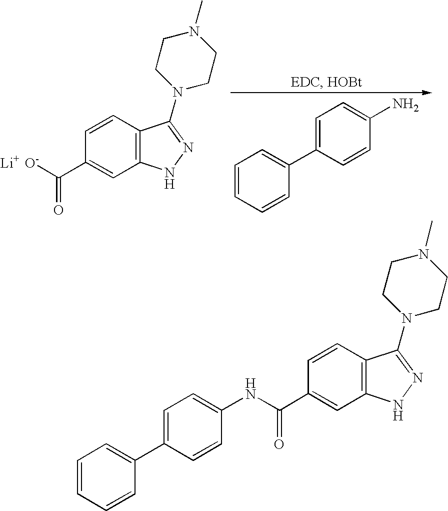

### 3-(4-Methyl-piperazin-1-yl)-1H-indazole-6-carboxylic acid biphenyl-4-ylamide

To a solution of lithium 3-(4-methyl-piperazin-1-yl)-1H-indazole-6-carboxylate (93 mg, 0.35 mmol) in anhydrous DMF (5 mL) was added EDC (81 mg, 0.42 mmol) and HOBt (57 mg, 0.42 mmol). The mixture was stirred for 10 min. then Et₃N (0.24 mL, 1.75 mmol) and 4-phenylaniline (56 mg, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (31 mg, 25%). m/z (M+H)=412.33.

### EXAMPLE 17

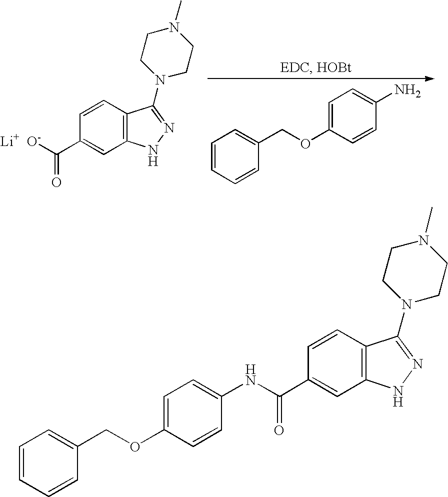

### 3-(4-Methyl-piperazin-1-yl)-1H-indazole-6-carboxylic acid (4-benzyloxy-phenyl)-amide

To a solution of lithium 3-(4-methyl-piperazin-1-yl)-1H-indazole-6-carboxylate (93 mg, 0.35 mmol) in anhydrous DMF (5 mL) was added EDC (81 mg, 0.42 mmol) and HOBt (57 mg, 0.42 mmol). The mixture was stirred for 10 min. then Et₃N (0.24 mL, 1.75 mmol) and 4-benzoxyaniline.HCl (78 mg, 0.33 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (35 mg, 27%). m/z (M+H)=442.54.

### EXAMPLE 18

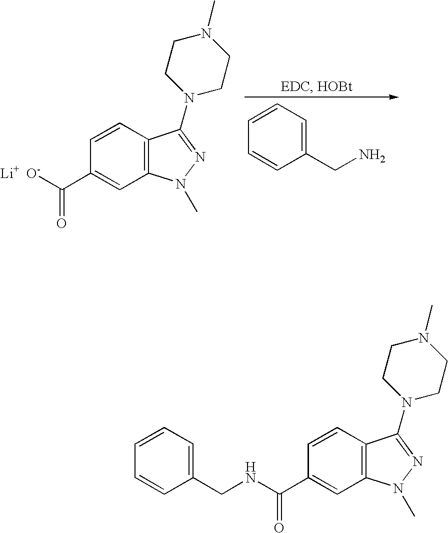

### 1-Methyl-3-(4-methyl-piperazin-1-yl)-1H-indazole-6-carboxylic acid benzylamide

To a solution of lithium 1-methyl-3-(4-methyl-piperazin-1-yl)-1H-indazole-6-carboxylate (88 mg, 0.32 mmol) in anhydrous DMF (5 mL) was added EDC (73 mg, 0.38 mmol) and HOBt (51 mg, 0.38 mmol). The mixture was stirred for 10 min. then Et₃N (0.11 mL, 0.80 mmol) and benzylamine (0.042 mL, 0.38 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (38 mg, 33%). m/z (M+H)=364.47.

### EXAMPLE 19

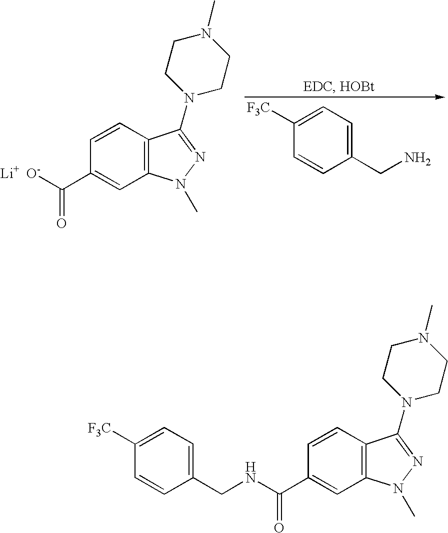

### 1-Methyl-3-(4-methyl-piperazin-1-yl)-1H-indazole-6-carboxylic acid 4-trifluoromethyl-benzylamide

To a solution of lithium 1-methyl-3-(4-methyl-piperazin-1-yl)-1H-indazole-6-carboxylate (88 mg, 0.32 mmol) in anhydrous DMF (5 mL) was added EDC (73 mg, 0.38 mmol) and HOBt (51 mg, 0.38 mmol). The mixture was stirred for 10 min. then Et₃N (0.11 mL, 0.80 mmol) and 4-trifluoromethylbenzylamine (0.054 mL, 0.38 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (42 mg, 30%). m/z (M+H)=378.49.

### EXAMPLE 20

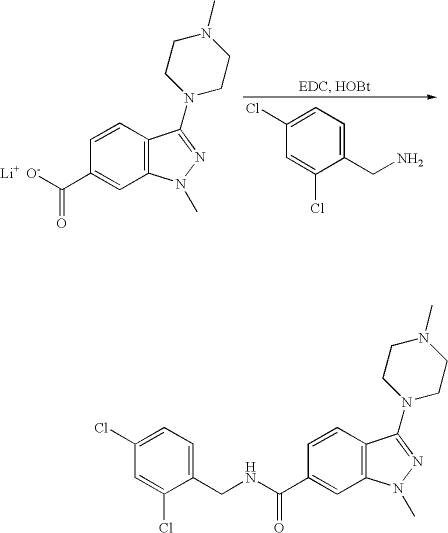

### 1-Methyl-3-(4-methyl-piperazin-1-yl)-1H-indazole-6-carboxylic acid 2,4-dichloro-benzylamide

To a solution of lithium 1-methyl-3-(4-methyl-piperazin-1-yl)-1H-indazole-6-carboxylate (88 mg, 0.32 mmol) in anhydrous DMF (5 mL) was added EDC (73 mg, 0.38 mmol) and HOBt (51 mg, 0.38 mmol). The mixture was stirred for 10 min. then Et₃N (0.11 mL, 0.80 mmol) and 2,4-dichlorobenzylamine (0.050 mL, 0.38 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (40 mg, 29%). m/z (M+H)=433.36.

### EXAMPLE 21

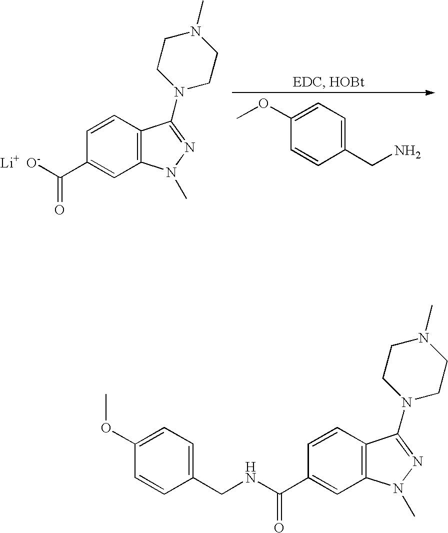

### 1-Methyl-3-(4-methyl-piperazin-1-yl)-1H-indazole-6-carboxylic acid 4-methoxy-benzylamide

To a solution of lithium 1-methyl-3-(4-methyl-piperazin-1-yl)-1H-indazole-6-carboxylate (88 mg, 0.32 mmol) in anhydrous DMF (5 mL) was added EDC (73 mg, 0.38 mmol) and HOBt (51 mg, 0.38 mmol). The mixture was stirred for 10 min. then Et₃N (0.11 mL, 0.80 mmol) and 4-methoxybenzylamine (0.050 mL, 0.38 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (32 mg, 25%). m/z (M+H)=394.49.

### EXAMPLE 22

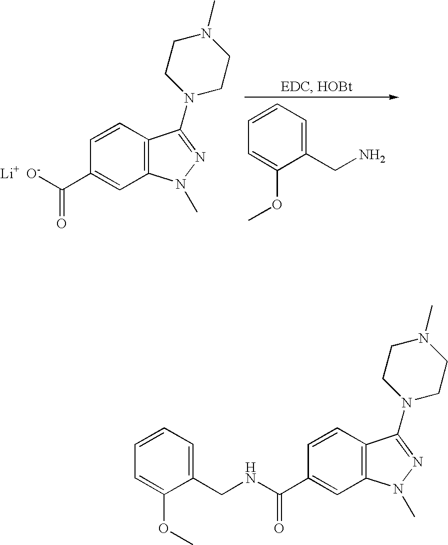

### 1-Methyl-3-(4-methyl-piperazin-1-yl)-1H-indazole-6-carboxylic acid 2-methoxy-benzylamide

To a solution of lithium 1-methyl-3-(4-methyl-piperazin-1-yl)-1H-indazole-6-carboxylate (88 mg, 0.32 mmol) in anhydrous DMF (5 mL) was added EDC (73 mg, 0.38 mmol) and HOBt (51 mg, 0.38 mmol). The mixture was stirred for 10 min. then Et₃N (0.11 mL, 0.80 mmol) and 4-methoxybenzylamine (0.050 mL, 0.38 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (30 mg, 24%). m/z (M+H)=394.49.

### EXAMPLE 23

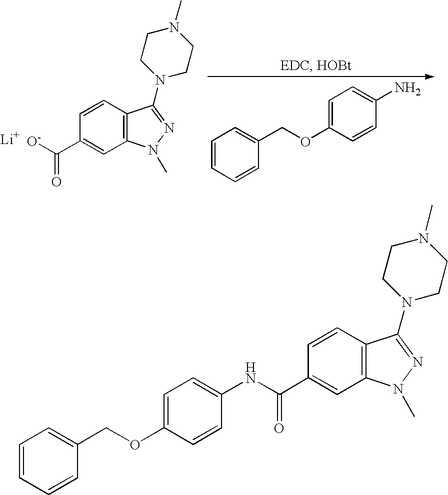

### (4-Benzyloxy-phenyl)-[1-methyl-3-(4-methyl-piperazin-1-yl)-1H-indazol-6-yl]-methanone

To a solution of lithium 1-methyl-3-(4-methyl-piperazin-1-yl)-1H-indazole-6-carboxylate (88 mg, 0.32 mmol) in anhydrous DMF (5 mL) was added EDC (73 mg, 0.38 mmol) and HOBt (51 mg, 0.38 mmol). The mixture was stirred for 10 min. then Et₃N (0.11 mL, 0.80 mmol) and 4-benzyloxyaniline.HCl (0.090 g, 0.38 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (32 mg, 27%). m/z (M+H)=456.56.

### EXAMPLE 24

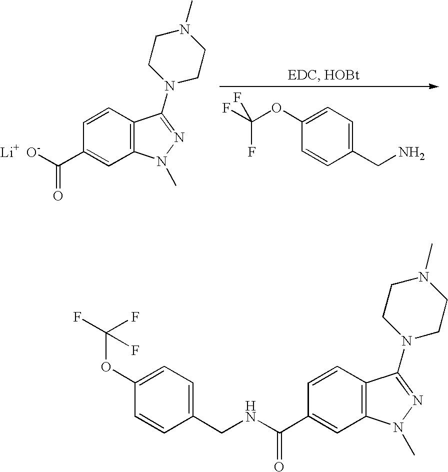

### 1-Methyl-3-(4-methyl-piperazin-1-yl)-1H-indazole-6-carboxylic acid 4-trifluoromethoxy-benzylamide

To a solution of lithium 1-methyl-3-(4-methyl-piperazin-1-yl)-1H-indazole-6-carboxylate (88 mg, 0.32 mmol) in anhydrous DMF (5 mL) was added EDC (73 mg, 0.38 mmol) and HOBt (51 mg, 0.38 mmol). The mixture was stirred for 10 min. then Et₃N (0.11 mL, 0.80 mmol) and 4-trifluoromethoxybenzylamine (0.058 mL, 0.38 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (28 mg, 20%). m/z (M+H)=448.46.

### EXAMPLE 25

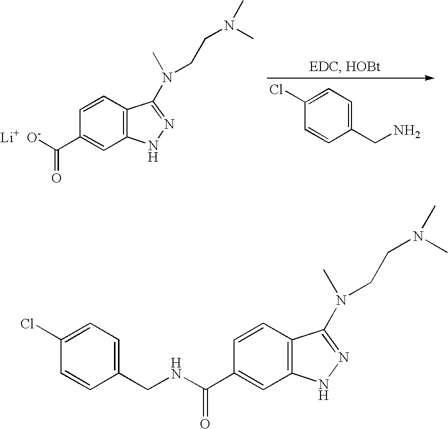

### 3-[(2-Dimethylamino-ethyl)-methyl-amino]-1H-indazole-6-carboxylic acid 4-chloro-benzylamide

To a solution of lithium 3-[(2-Dimethylamino-ethyl)-methyl-amino]-1H-indazole-6-carboxylate (77 mg, 0.29 mmol) in anhydrous DMF (5 mL) was added EDC (140 mg, 0.73 mmol) and HOBt (47 mg, 0.35 mmol). The mixture was stirred for 10 min. then Et₃N (0.16 mL, 0.1.16 mmol) and 4-chlorobenzylamine (0.040 mL, 0.32 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (37 mg, 33%). m/z (M+H)=386.90.

### EXAMPLE 26

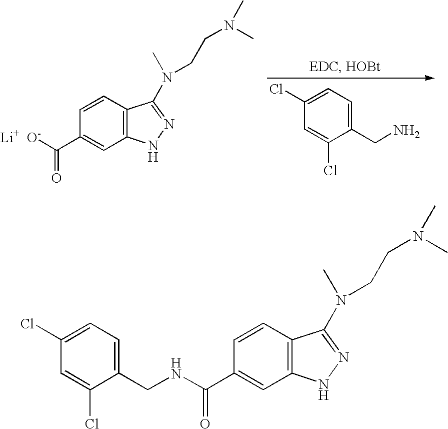

### 3-[(2-Dimethylamino-ethyl)-methyl-amino]-1H-indazole-6-carboxylic acid 2,4-dichloro-benzylamide

To a solution of lithium 3-[(2-Dimethylamino-ethyl)-methyl-amino]-1H-indazole-6-carboxylate (77 mg, 0.29 mmol) in anhydrous DMF (5 mL) was added EDC (140 mg, 0.73 mmol) and HOBt (47 mg, 0.35 mmol). The mixture was stirred for 10 min. then Et₃N (0.16 mL, 0.1.16 mmol) and 2,4-dichlorobenzylamine (0.040 mL, 0.32 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (30 mg, 25%). m/z (M+H)=421.35.

### EXAMPLE 27

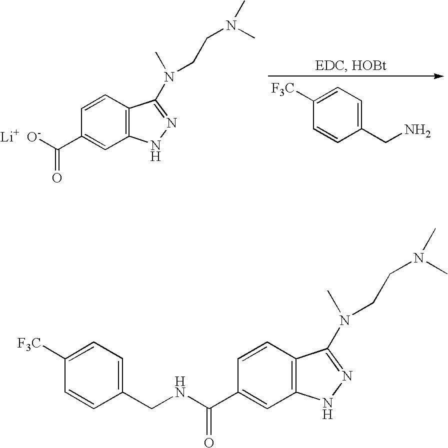

### 3-[(2-Dimethylamino-ethyl)-methyl-amino]-1H-indazole-6-carboxylic acid 4-trifluoromethyl-benzylamide

To a solution of lithium 3-[(2-Dimethylamino-ethyl)-methyl-amino]-1H-indazole-6-carboxylate (77 mg, 0.29 mmol) in anhydrous DMF (5 mL) was added EDC (140 mg, 0.73 mmol) and HOBt (47 mg, 0.35 mmol). The mixture was stirred for 10 min. then Et₃N (0.16 mL, 0.1.16 mmol) and 4-trifluoromethylbenzylamine (0.046 mL, 0.32 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (32 mg, 26%). m/z (M+H)=368.45.

### EXAMPLE 28

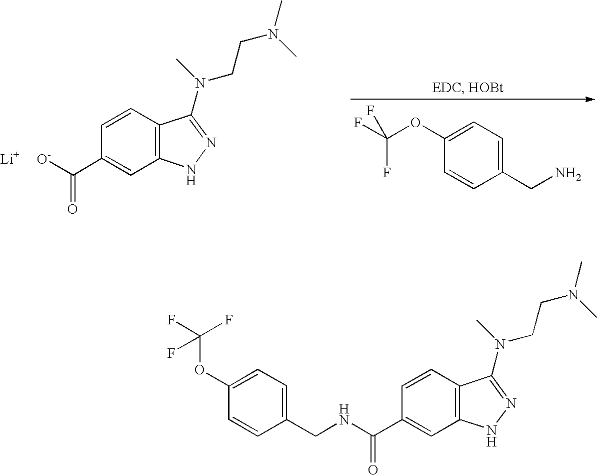

### 3-[(2-Dimethylamino-ethyl)-methyl-amino]-1H-indazole-6-carboxylic acid 4-trifluoromethoxy-benzylamide

To a solution of lithium 3-[(2-Dimethylamino-ethyl)-methyl-amino]-1H-indazole-6-carboxylate (77 mg, 0.29 mmol) in anhydrous DMF (5 mL) was added EDC (140 mg, 0.73 mmol) and HOBt (47 mg, 0.35 mmol). The mixture was stirred for 10 min. then Et₃N (0.16 mL, 0.1.16 mmol) and 4-trifluoromethoxybenzylamine (0.049 mL, 0.32 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (37 mg, 29%). m/z (M+H)=436.45.

### EXAMPLE 29

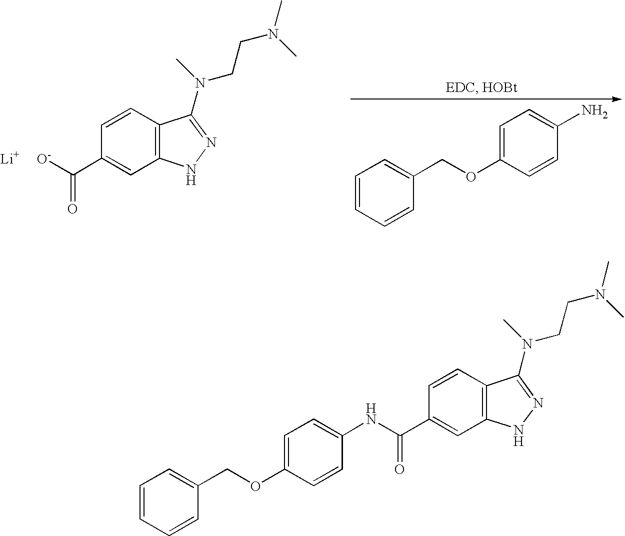

### 3-[(2-Dimethylamino-ethyl)-methyl-amino]-1H-indazole-6-carboxylic acid (4-benzyloxy-phenyl)-amide

To a solution of lithium 3-[(2-Dimethylamino-ethyl)-methyl-amino]-1H-indazole-6-carboxylate (77 mg, 0.29 mmol) in anhydrous DMF (5 mL) was added EDC (140 mg, 0.73 mmol) and HOBt (47 mg, 0.35 mmol). The mixture was stirred for 10 min. then Et₃N (0.16 mL, 0.1.16 mmol) and 4-benzyloxyaniline.HCl (0.076 g, 0.32 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (35 mg, 27%). m/z (M+H)=444.45.

### EXAMPLE 30

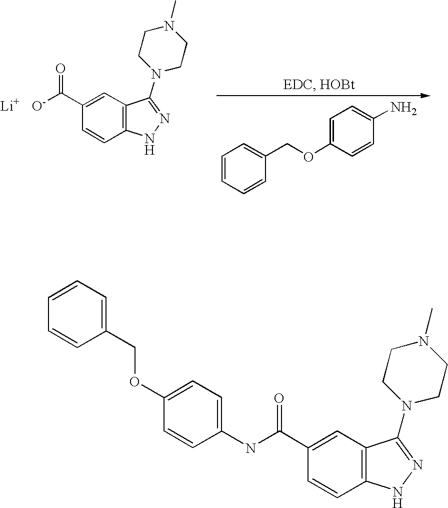

### 3-(4-Methyl-piperazin-1-yl)-1H-indazole-5-carboxylic acid (4-benzyloxy-phenyl)-amide

To a solution of lithium 3-(4-Methyl-piperazin-1-yl)-1H-indazole-5-carboxylate (117 mg, 0.44 mmol) in anhydrous DMF (5 mL) was added EDC (102 mg, 0.53 mmol) and HOBt (71 mg, 0.53 mmol). The mixture was stirred for 10 min. then Et₃N (0.18 mL, 1.32 mmol) and 4-benzyloxyaniline.HCl (0.114 g, 0.48 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (80 mg, 41%). m/z (M+H)=442.54.

### EXAMPLE 31

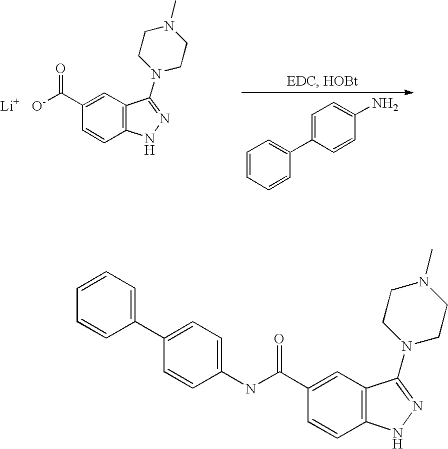

### 3-(4-Methyl-piperazin-1-yl)-1H-indazole-5-carboxylic acid biphenyl-4-ylamide

To a solution of lithium 3-(4-Methyl-piperazin-1-yl)-1H-indazole-5-carboxylate (117 mg, 0.44 mmol) in anhydrous DMF (5 mL) was added EDC (102 mg, 0.53 mmol) and HOBt (71 mg, 0.53 mmol). The mixture was stirred for 10 min. then Et₃N (0.18 mL, 1.32 mmol) and 4-phenylaniline (0.081 g, 0.48 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (25 mg, 14%). m/z (M+H)=413.54.

### EXAMPLE 32

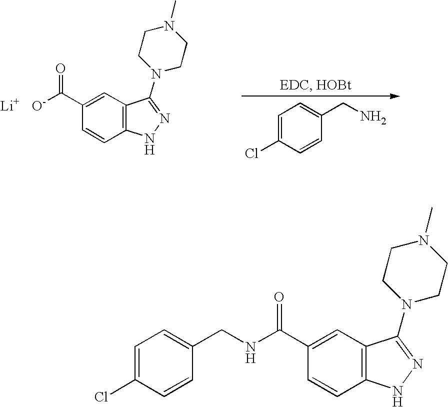

### 3-(4-Methyl-piperazin-1-yl)-1H-indazole-5-carboxylic acid 4-chloro-benzylamide

To a solution of lithium 3-(4-Methyl-piperazin-1-yl)-1H-indazole-5-carboxylate (117 mg, 0.44 mmol) in anhydrous DMF (5 mL) was added EDC (102 mg, 0.53 mmol) and HOBt (71 mg, 0.53 mmol). The mixture was stirred for 10 min. then Et₃N (0.18 mL, 1.32 mmol) and 4-chlorobenzylamine (0.060 mL, 0.48 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (21 mg, 21%). m/z (M+H)=384.88.

### EXAMPLE 33

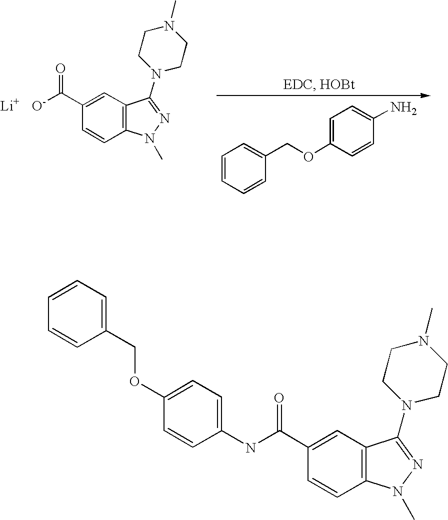

### 1-Methyl-3-(4-methyl-piperazin-1-yl)-1H-indazole-5-carboxylic acid (4-benzyloxy-phenyl)-amide

To a solution of lithium 3-(4-Methyl-piperazin-1-yl)-1H-indazole-5-carboxylate (67 mg, 0.24 mmol) in anhydrous DMF (5 mL) was added EDC (56 mg, 0.29 mmol) and HOBt (39 mg, 0.29 mmol). The mixture was stirred for 10 min. then Et₃N (0.10 mL, 0.72 mmol) and 4-benzyloxyaniline.HCl (0.062 g, 0.26 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (27 mg, 25%). m/z (M+H)=456.54.

### EXAMPLE 34

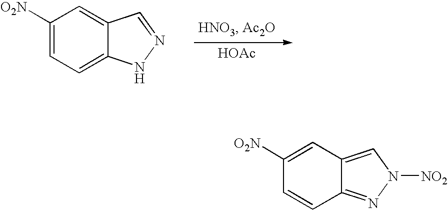

### 2,5-Dinitro-2H-indazole

A suspension of 5-nitroindazole (869 mg, 5.33 mmol) in acetic acid (7.5 mL) was poured into a solution of HNO₃ (1.5 mL) and Ac₂O (3.5 mL) that was cooled to −5° C. After 2 min. the mixture was poured onto ice and stirred for 30 min. The precipitate was filtered and air dried. The material was used without further purification.

### EXAMPLE 35

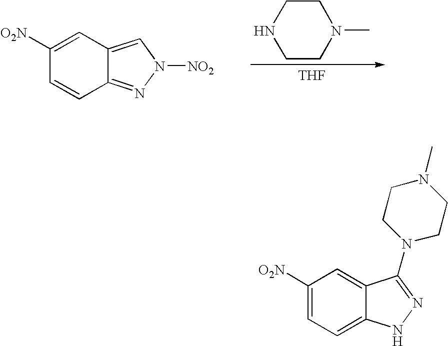

### 3-(4-Methyl-piperazin-1-yl)-5-nitro-1H-indazole

To a solution of 2,5-Dinitro-2H-indazole (400 mg, 1.92 mmol) in THF (15 mL) was added 1-methylpiperazine (0.40 mL, 3.62 mmol). The mixture was stirred for 15 hr. then concentrated in vacuo. The residue was diluted with ethyl acetate (50 mL) and washed with sat. aq. sodium bicarbonate (25 mL). The aqueous layer was extracted with ethyl acetate (25 mL). The combined organic layers were dried over anhydrous sodium sulfate and concentrated in vacuo. Chromatography on silica yielded the title compound (242 mg, 49%). m/z (M+H)=262.29.

### EXAMPLE 36

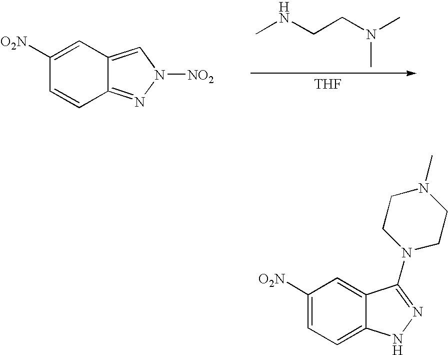

### N,N,N′-Trimethyl-N′-(5-nitro-1H-indazol-3-yl)-ethane-1,2-diamine

To a solution of 2,5-Dinitro-2H-indazole (400 mg, 1.92 mmol) in THF (15 mL) was added N,N,N′-trimethylethylenediamine (0.40 mL, 3.62 mmol). The mixture was stirred for 15 hr. then concentrated in vacuo. The residue was diluted with ethyl acetate (50 mL) and washed with sat. aq. sodium bicarbonate (25 mL). The aqueous layer was extracted with ethyl acetate (25 mL). The combined organic layers were dried over anhydrous sodium sulfate and concentrated in vacuo. Chromatography on silica yielded the title compound (242 mg, 49%). m/z (M+H)=264.30.

### EXAMPLE 37

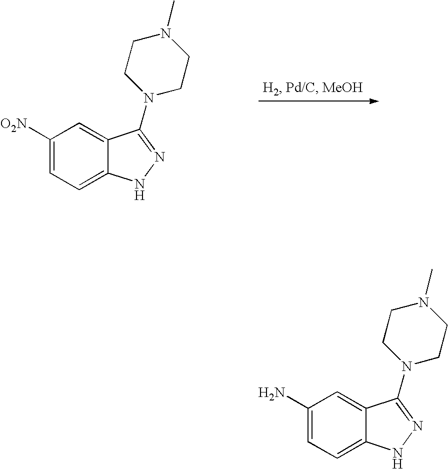

### 3-(4-Methyl-piperazin-1-yl)-1H-indazol-5-ylamine

A solution of 3-(4-Methyl-piperazin-1-yl)-5-nitro-1H-indazole (422 mg, 1.61 mmol) and 10% Pd/C (300 mg) in MeOH (40 mL) was shaken on a Parr hydrogenator under H₂ (50 psi) for 1 hr. The mixture was filtered through celite and the filtrate was concentrated to yield the title compound (369 mg, 99%). m/z (M+H)=248.35.

### EXAMPLE 38

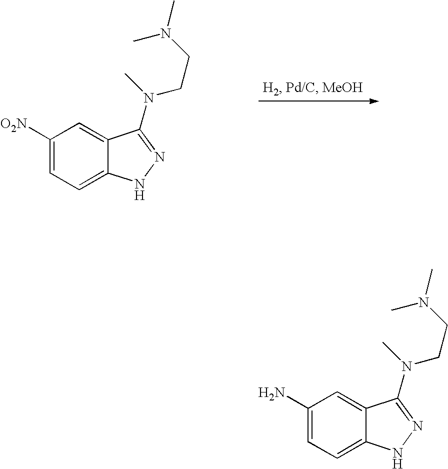

### N³-(2-Dimethylamino-ethyl)-N³-methyl-1H-indazole-3,5-diamine

A solution of N,N,N′-Trimethyl-N′-(5-nitro-1H-indazol-3-yl)-ethane-1,2-diamine (337 mg, 1.28 mmol) and 10% Pd/C (300 mg) in MeOH (40 mL) was shaken on a Parr hydrogenator under H₂ (50 psi) for 1 h. The mixture was filtered through celite and the filtrate was concentrated to yield the title compound (277 mg, 93%). m/z (M+H)=234.30.

### EXAMPLE 39

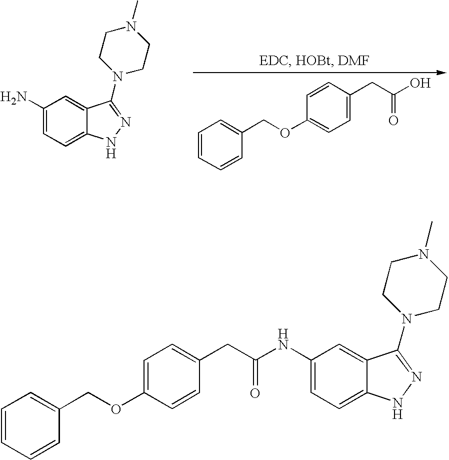

### 2-(4-Benzyloxy-phenyl)-N-[3-(4-methyl-piperazin-1-yl)-1H-indazol-5-yl]-acetamide

To a solution of 4-phenoxyphenylacetic acid (58 mg, 0.24 mmol) in anhydrous DMF (5 mL) was added EDC (50 mg, 0.26 mmol) and HOBt (35 mg, 0.26 mmol). The mixture was stirred for 10 min. then Et₃N (0.06 mL, 0.43 mmol) and 3-(4-Methyl-piperazin-1-yl)-1H-indazol-5-ylamine (0.050 g, 0.22 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (52 mg, 52%). m/z (M+H)=472.61.

### EXAMPLE 40

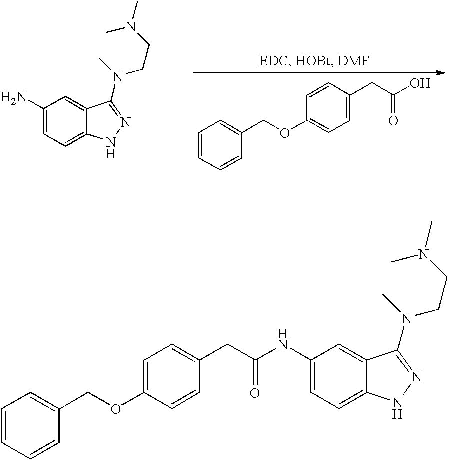

### 2-(4-Benzyloxy-phenyl)-N-{3-[(2-dimethylamino-ethyl)-methyl-amino]-1H-indazol-5-yl}-acetamide

To a solution of 4-phenoxyphenylacetic acid (58 mg, 0.24 mmol) in anhydrous DMF (5 mL) was added EDC (50 mg, 0.26 mmol) and HOBt (35 mg, 0.26 mmol). The mixture was stirred for 10 min. then Et₃N (0.06 mL, 0.43 mmol) and N³-(2-dimethylaminoethyl)-N³-methyl-1H-indazole-3,5-diamine (0.050 g, 0.22 mmol) were added. The mixture was heated at 50° C. for 15 hr. The mixture was allowed to cool, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (50 mg, 52%). In/z (M+H)=458.54.

### EXAMPLE 41

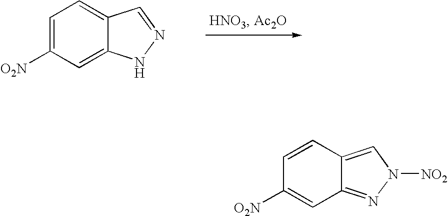

### 2,6-Dinitro-2H-indazole

A suspension of 6-Nitro-1H-Indazole (2.45 g, 15.0 mmol) in acetic acid (18.4 mL) was poured into a solution of HNO₃ (3.7 mL) and Ac₂O (8.6 mL) that was cooled to −5° C. After 5 min. the mixture was poured onto ice and stirred for 30 min. The precipitate was filtered, washed with water, and air dried. The material was used without further purification.

### EXAMPLE 42

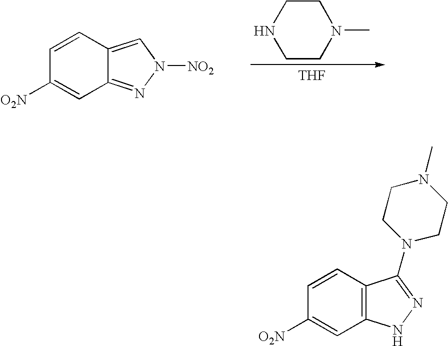

### 3-(4-Methyl-piperazin-1-yl)-6-nitro-1H-indazole

To a solution of 2,6-dinitro-2H-indazole (1.56 g, 7.5 mmol) in THF (25 mL) was added 1-methylpiperazine (1.65 mL, 15.0 mmol). The mixture was stirred for 15 hr. then concentrated in vacuo. The residue was diluted with ethyl acetate (75 mL) and washed with sat. aq. sodium bicarbonate (30 mL). The aqueous layer was extracted with ethyl acetate (30 mL). The combined organic layers were dried over anhydrous sodium sulfate and concentrated in vacuo. Chromatography on silica yielded the title compound (1.23 g, 62.8%). m/z (M+H)=262.29.

### EXAMPLE 43

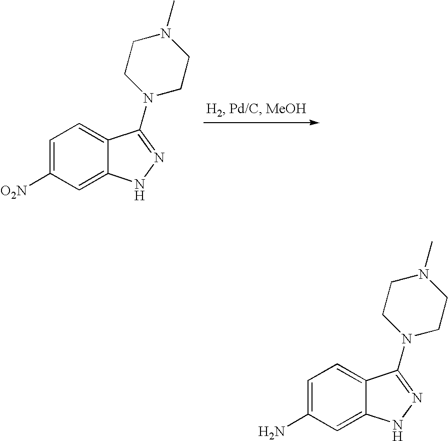

### 3-(4-Methyl-piperazin-1-yl)-1H-indazol-6-ylamine

A solution of 3-(4-Methyl-piperazin-1-yl)-6-nitro-1H-indazole (1.22 g, 4.67 mmol) and 10% Pd/C (500 mg) in MeOH (100 mL) was hydrogenated at atmospheric pressure for 2 hr. The mixture was filtered through celite and the filtrate was concentrated to yield the title compound (1.07 g, 99%). m/z (M+H)=232.30.

### EXAMPLE 44

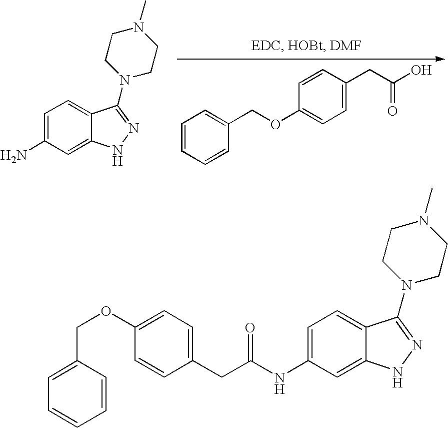

### 2-(4-Benzyloxy-phenyl)-N-[3-(4-methyl-piperazin-1-yl)-1H-indazol-6-yl]-acetamide

To a solution of 4-phenoxyphenylacetic acid (58 mg, 0.24 mmol) in anhydrous DMF (5 mL) was added EDC (50 mg, 0.26 mmol) and HOBt (35 mg, 0.26 mmol). The mixture was stirred for 10 min. then Et₃N (0.046 mL, 0.33 mmol) and 3-(4-Methyl-piperazin-1-yl)-1H-indazol-6-ylamine (0.050 g, 0.22 mmol) were added. The mixture was stirred at room temperature for 2 hr., then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (34 mg, 34%). m/z (M+H)=456.56.

### EXAMPLE 45

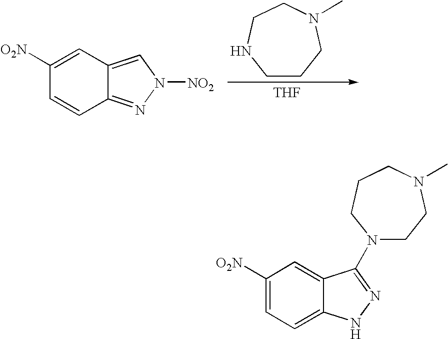

### 3-(4-Methyl-homopiperazin-1-yl)-5-nitro-1H-indazole

To a solution of 2,5-Dinitro-2H-indazole (2.6 g, 12.5 mmol) in THF (50 mL) was added 1-methylhomopiperazine (3.1 mL, 25 mmol). The mixture was stirred for 15 hr. then concentrated in vacuo. The residue was diluted with ethyl acetate (100 mL) and washed with sat. aq. sodium bicarbonate (50 mL). The aqueous layer was extracted with ethyl acetate (50 mL). The combined organic layers were dried over anhydrous sodium sulfate and concentrated in vacuo. Chromatography on silica yielded the title compound (1.39 g, 40.4%). m/z (M+H)=276.31.

### EXAMPLE 46

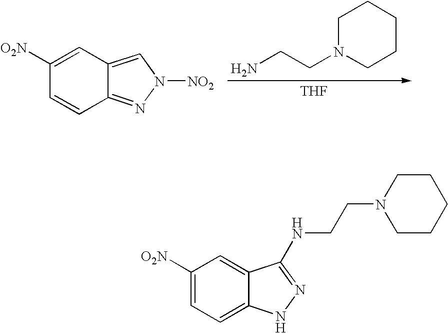

### 3-(2-Piperidin-1-yl-ethylamino)-5-nitro-1H-indazole

To a solution of 2,5-Dinitro-2H-indazole (2.6 g, 12.5 mmol) in THF (50 mL) was added 1-(2-aminoethyl)piperidine (3.6 mL, 25 mmol). The mixture was stirred for 15 hr. then concentrated in vacuo. The residue was diluted with ethyl acetate (100 mL) and washed with sat. aq. sodium bicarbonate (50 mL). The aqueous layer was extracted with ethyl acetate (50 mL). The combined organic layers were dried over anhydrous sodium sulfate and concentrated in vacuo. Chromatography on silica yielded the title compound (0.50 g, 14%). m/z (M+H)=290.34.

### EXAMPLE 47

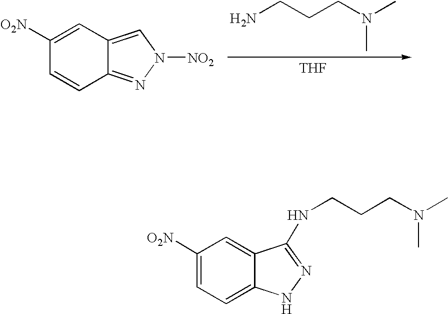

### N,N-Dimethyl-N′-(5-nitro-1H-indazol-3-yl)-propane-1,3-diamine

To a solution of 2,5-Dinitro-2H-indazole (2.6 g, 12.5 mmol) in THF (50 mL) was added 3-(dimethylamino)-1-propylamine (3.1 mL, 25 mmol). The mixture was stirred for 15 hr. then concentrated in vacuo. The residue was diluted with ethyl acetate (100 mL) and washed with sat. aq. sodium bicarbonate (50 mL). The aqueous layer was extracted with ethyl acetate (50 mL). The combined organic layers were dried over anhydrous sodium sulfate and concentrated in vacuo. Chromatography on silica yielded the title compound (1.11 g, 38%). m/z (M+H)=264.30.

### EXAMPLE 48

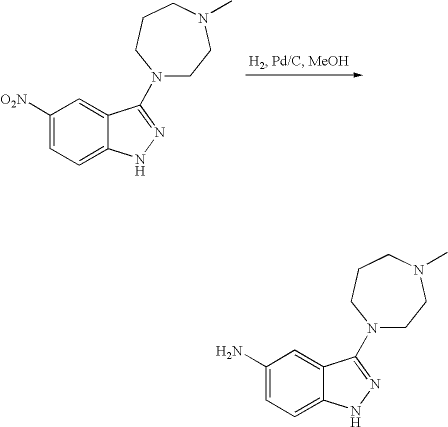

### 3-(4-Methyl-homopiperazin-1-yl)-1H-indazol-5-ylamine

A solution of 3-(4-Methyl-homopiperazin-1-yl)-5-nitro-1H-indazole (1.39 g, 5.05 mmol) and 10% Pd/C (600 mg) in MeOH (100 mL) was hydrogenated at atmospheric pressure for 5 hr. The mixture was filtered through celite and the filtrate was concentrated to yield the title compound (1.05 g, 84.9%). m/z (M+H)=246.33.

### EXAMPLE 49

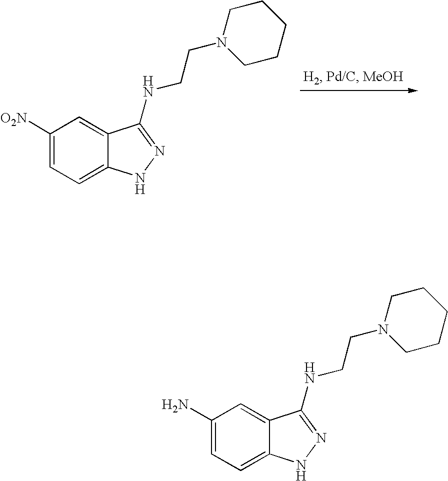

### 3-(2-Piperidin-1-yl-ethylamino)-1H-indazole-5-ylamine

A solution of 3-(2-Piperidin-1-yl-ethylamino)-5-nitro-1H-indazole (0.50 g, 1.73 mmol) and 10% Pd/C (400 mg) in MeOH (40 mL) was hydrogenated at atmospheric pressure for 5 hr. The mixture was filtered through celite and the filtrate was concentrated to yield the title compound (0.35 g, 78.1%). m/z (M+H)=260.36.

### EXAMPLE 50

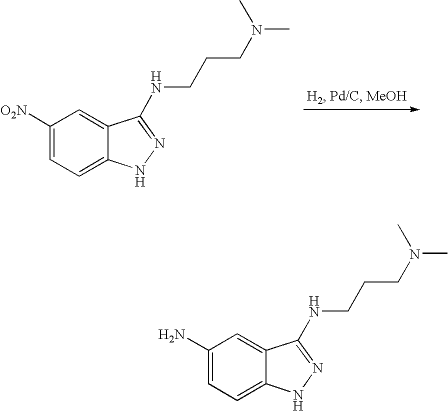

### N,N-Dimethyl-N′-(5-amino-1H-indazol-3-yl)-propane-1,3-diamine

A solution of N,N-Dimethyl-N-(5-nitro-1H-indazol-3-yl)-propane-1,3-diamine (1.11 g, 4.22 mmol) and 10% Pd/C (600 mg) in MeOH (100 mL) was hydrogenated at atmospheric pressure for 5 hr. The mixture was filtered through celite and the filtrate was concentrated to yield the title compound (0.81 g, 83.4%). m/z (M+H)=234.32.

### EXAMPLE 51

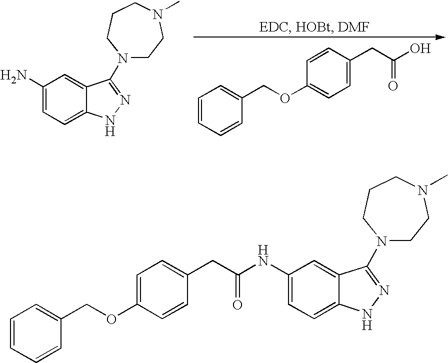

### 2-(4-Benzyloxy-phenyl)-N-[3-(4-methyl-homopiperazin-1-yl)-1H-indazol-5-yl]-acetamide

To a solution of 4-phenoxyphenylacetic acid (54 mg, 0.22 mmol) in anhydrous DMF (5 mL) was added EDC (47 mg, 0.24 mmol) and HOBt (33 mg, 0.24 mmol). The mixture was stirred for 10 min. then Et₃N (0.043 mL, 0.31 mmol) and 3-(4-methyl-homopiperazin-1-yl)-1H-indazol-5-ylamine (50 mg, 0.22 mmol) were added. The mixture was stirred at room temperature for 4 hr. then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (66 mg, 69.0%). m/z (M+H)=470.59.

### EXAMPLE 52

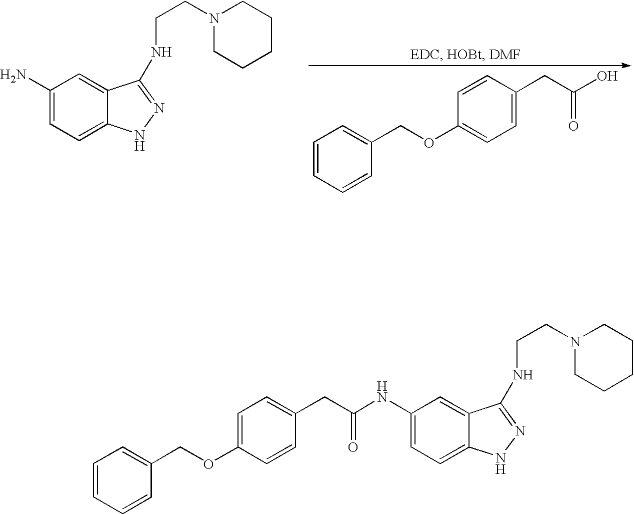

### 2-(4-Benzyloxy-phenyl)-N-[3-(2-Piperidin-1-yl-ethylamino)-1H-indazol-5-yl]-acetamide

To a solution of 4-phenoxyphenylacetic acid (52 mg, 0.21 mmol) in anhydrous DMF (5 mL) was added EDC (45 mg, 0.23 mmol) and HOBt (31 mg, 0.23 mmol). The mixture was stirred for 10 min. then Et₃N (0.040 mL, 0.29 mmol) and 3-(2-Piperidin-1-yl-ethylamino)-1H-indazole-5-ylamine (50 mg, 0.19 mmol) were added. The mixture was stirred at room temperature for 4 hr. then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (51 mg, 54.7%). m/z (M+H)=484.62.

### EXAMPLE 53

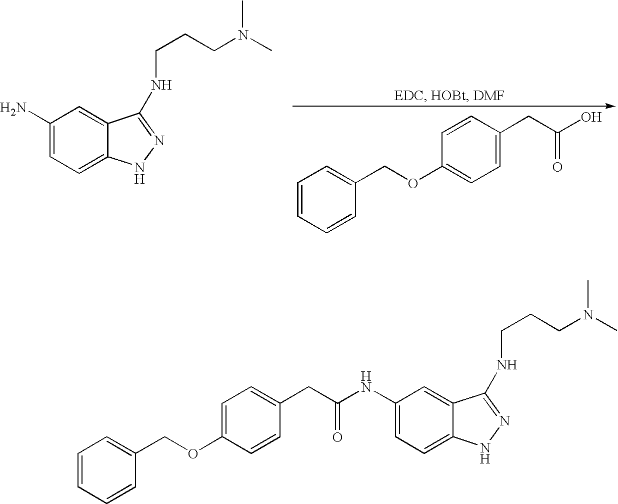

### 2-(4-Benzyloxy-phenyl)-N-[3-(3-dimethylaminopropylamino)-1H-indazol-5-yl]-acetamide

To a solution of 4-phenoxyphenylacetic acid (57 mg, 0.24 mmol) in anhydrous DMF (5 mL) was added EDC (50 mg, 0.26 mmol) and HOBt (35 mg, 0.26 mmol). The mixture was stirred for 10 min. then Et₃N (0.045 mL, 0.32 mmol) and N,N-Dimethyl-N′-(5-amino-1H-indazol-3-yl)-propane-1,3-diamine (50 mg, 0.22 mmol) were added. The mixture was stirred at room temperature for 4 hr. then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (71 mg, 72.3%). m/z (M+H)=458.58.

### EXAMPLE 54

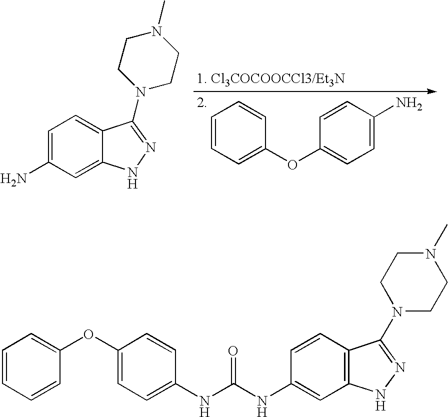

### {[3-(4-Methylpiperazinyl)(1H-indazol-6-yl0]amino}-N-(4-phenoxyphenyl)carboxamide

Triphosgene (33 mg, 0.108 mmol) was added to suspension of 3-(4-Methyl-piperazin-1-yl)-1H-indazol-6-ylamine (75 mg, 0.324 mmol) in THF (12 mL) at 0° C. After 5 min, triethylamine (0.135 mL, 0.972 mmol) was added and mixture was allowed to warm to 17° C. for 15 min. The reaction mixture was cooled down to 0° C. again, and a solution 4-phenoxyphenylamine (60 mg, 0.324 mmol) in 2 mL THF was added. The mixture was stirred for 15 min at 0° C., and 2 hr. at 20° C. The resulting mixture was filtered, washed with THF (10 mL), and the filtrate was concentrated in in vacuo. Chromatography on silica yielded the title compound (59 mg, 41.2%). m/z (M+H)=443.52.

### EXAMPLE 55

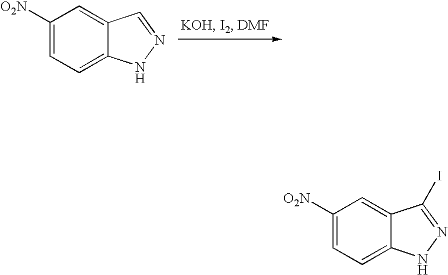

### 3-Iodo-5-nitro-1H-indazole

To a solution of 5-nitroindazole (1.0 g, 6.13 mmol) in DMF (25 mL) was added KOH (1.27 g, 22.7 mmol) and iodine (3.07 g, 12.1 mmol). The mixture was stirred for 1 hr. then poured into 10% Na₂S₂O₅ (250 mL). The mixture was stirred for 15 min. then filtered. The solid was dried to yield the title compound (1.56 g, 88%). m/z (M+H)=290.03.

### EXAMPLE 56

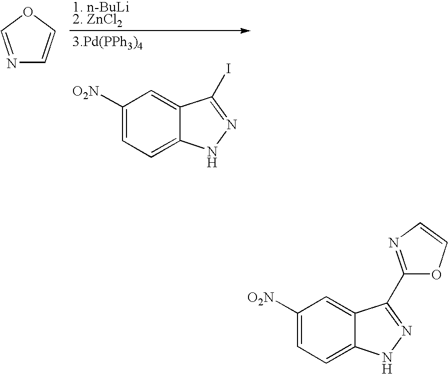

### 5-Nitro-3-oxazol-2-yl-1H-indazole

A solution of oxazole (1.1 mL, 17.3 mmol) in dry THF (25 mL) under an atmosphere of nitrogen was cooled to −78° C. To this mixture was added n-BuLi (1.6M in hexanes, 11.0 mL, 17.3 mmol) and the resulting yellow solution was allowed to stir for 30 min. A solution of ZnCl₂ (0.5M in THF, 35 mL, 17.3 mmol) was added and the mixture was allowed to warm to 0° C. and stirred for 15 min. Pd(PPh₃)₄ and 3-iodo-5-nitro-1H-indazole were added and the mixture was heated at reflux for 2 hr. The mixture was allowed to cool, diluted with ethyl acetate (100 mL) and washed with water (100 mL) and 2N HCl (50 mL). The organic layer was dried over anhydrous sodium sulfate and concentrated in vacuo. The material was purified by chromatography to yield the title compound (400 mg, 50%). m/z (M+H)=231.18.

### EXAMPLE 57

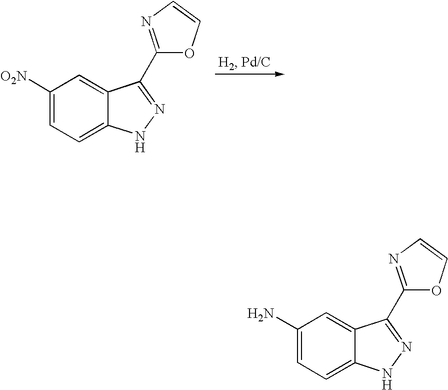

### 3-Oxazol-2-yl-1H-indazol-5-ylamine

A solution of 5-nitro-3-oxazol-2-yl-1H-indazole (373 mg, 1.62 mmol) and 10% Pd/C (150 mg) in MeOH (25 mL) was hydrogenated at atmospheric pressure for 5 hr. The mixture was filtered through celite and the filtrate was concentrated to yield the title compound (280 mg, 86%). m/z (M+H)=201.20.

### EXAMPLE 58

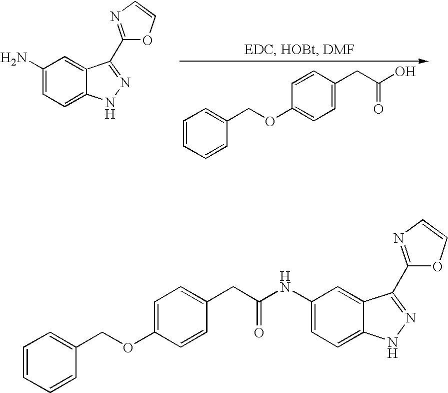

### 2-(4-Benzyloxy-phenyl)-N-(3-oxazol-2-yl-1H-indazol-5-yl)-acetamide

To a solution of 4-phenoxyphenylacetic acid (41 mg, 0.17 mmol) in anhydrous DMF (5 mL) was added EDC (34 mg, 0.18 mmol) and HOBt (24 mg, 0.18 mmol). The mixture was stirred for 10 min. then Et₃N (0.040 mL, 0.30 mmol) and 3-Oxazol-2-yl-1H-indazol-5-ylamine (30 mg, 0.15 mmol) were added. The mixture was stirred at room temperature for 4 hr. then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (30 mg, 47%). m/z (M+H)=425.46.

### EXAMPLE 59

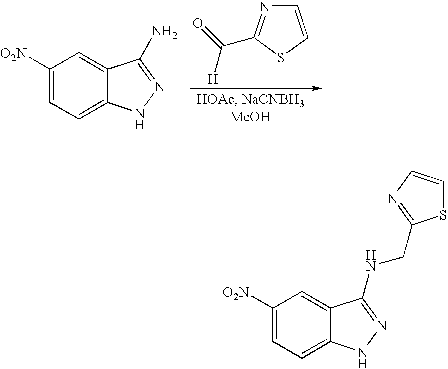

### (5-Nitro-1H-indazol-3-yl)-thiazol-2-ylmethyl-amine

To a suspension of 5-nitro-1H-indazol-3-ylamine (500 mg, 2.81 mmol) in MeOH (25 mL) was added HOAc (0.5 mL) and 2-thiazolecarboxaldehyde (0.25 mL, 2.81 mmol). The mixture was stirred for 10 min then NaCNBH₃ (354 mg, 5.62 mmol) was added. The reaction was allowed to stir 24 hr. then concentrated in vacuo. The residue was diluted with ethyl acetate (50 mL) and washed with NaHCO₃ (50 mL). The organic layer was dried over anhydrous sodium sulfate and concentrated in vacuo. Chromatography on silica yielded the title compound (299 mg, 39%). m/z (M+H)=276.29.

### EXAMPLE 60

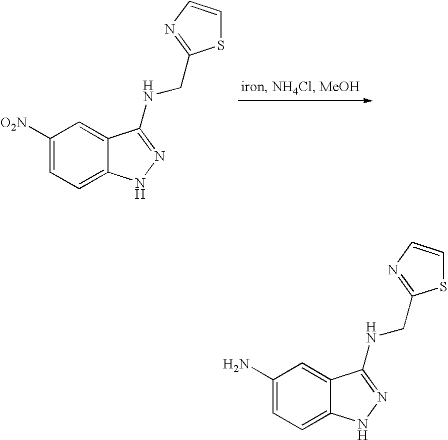

### N³-Thiazol-2-ylmethyl-1H-indazole-3,5-diamine

A mixture of (5-nitro-1H-indazol-3-yl)-thiazol-2-ylmethyl-amine (200 mg, 0.73 mmol) in sat. ammonium chloride (10 mL) and MeOH (10 mL) was heated at reflux for 3 hr. The mixture was filtered through celite and concentrated. The product was used without further purification.

### EXAMPLE 61

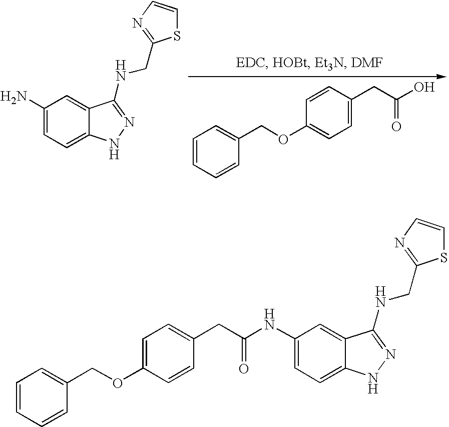

### 2-(4-Benzyloxy-phenyl)-N-{3-[(thiazol-2-ylmethyl)-amino]-1H-indazol-5-yl}-acetamide

To a solution of 4-phenoxyphenylacetic acid (99 mg, 0.41 mmol) in anhydrous DMF (5 mL) was added EDC (94 mg, 0.49 mmol) and HOBt (66 mg, 0.49 mmol). The mixture was stirred for 10 min. then Et₃N (0.090 mL, 0.62 mmol) and N³-Thiazol-2-ylmethyl-1H-indazole-3,5-diamine (100 mg, 0.41 mmol) were added. The mixture was stirred at room temperature for 4 hr. then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (30 mg, 47%). m/z (M+H)=470.57.

### EXAMPLE 62

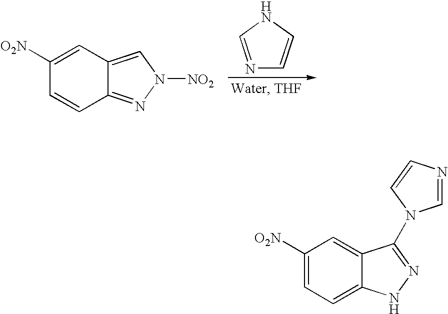

### 3-Imidazol-1-yl-5-nitro-1H-indazole

To a solution of 2,5-Dinitro-2H-indazole (500 mg, 2.40 mmol) in water (15 mL) and THF (10 mL) was added imidazole (322 mg, 4.8 mmol). The mixture was stirred for 15 hr. then concentrated in vacuo. The residue was triturated with water (50 mL) and filtered and dried to yield the title compound (269 mg, 49%). m/z (M+H)=230.68.

### EXAMPLE 63

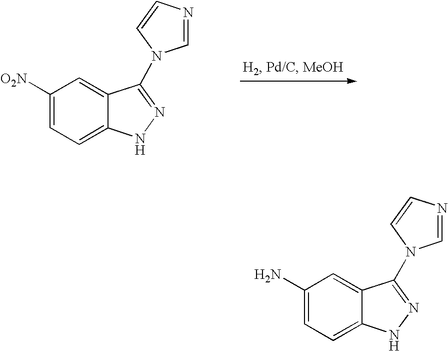

### 3-Imidazol-1-yl-1H-indazol-5-ylamine

A solution of 3-Imidazol-1-yl-5-nitro-1H-indazole (200 mg, 0.87 mmol) and 10% Pd/C (150 mg) in MeOH (25 mL) was hydrogenated at atmospheric pressure for 5 h. The mixture was filtered through celite and the filtrate was concentrated to yield the title compound (121 mg, 70%). m/z (M+H)=200.22.

### EXAMPLE 64

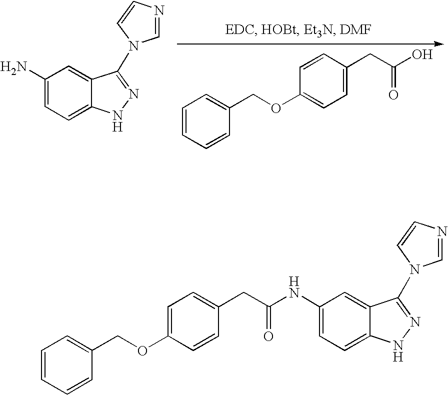

### 2-(4-Benzyloxy-phenyl)-N-(3-imidazol-1-yl-1H-indazol-5-yl)-acetamide

To a solution of 4-phenoxyphenylacetic acid (31 mg, 0.13 mmol) in anhydrous DMF (5 mL) was added EDC (31 mg, 0.16 mmol) and HOBt (22 mg, 0.16 mmol). The mixture was stirred for 10 min. then Et₃N (0.030 mL, 0.20 mmol) and 3-Imidazol-1-yl-1H-indazol-5-ylamine (25 mg, 0.13 mmol) were added. The mixture was stirred at room temperature for 4 hr. then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (14 mg, 25%). m/z (M+H)=424.48.

### EXAMPLE 65

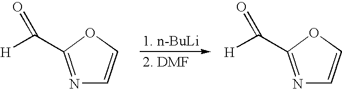

### Oxazole-2-carbaldehyde

To a solution of oxazole (2.0 g, 29.0 mmol) in dry THF under an atmosphere of nitrogen, cooled to −78° C. was added n-BuLi (1.6M in hexanes, 20.0 mL, 31.9 mmol). The mixture was stirred for 30 min and dry DMF (22.3 mL, 290 mmol) was added. The solution was allowed to warm to room temperature and stirred for 5 hr. The mixture was diluted with ethyl acetate (100 mL) and washed with 2N HCl. The organic layer was dried over anhydrous sodium sulfate and concentrated in vacuo to yield the title compound. The material was used without further purification.

### EXAMPLE 66

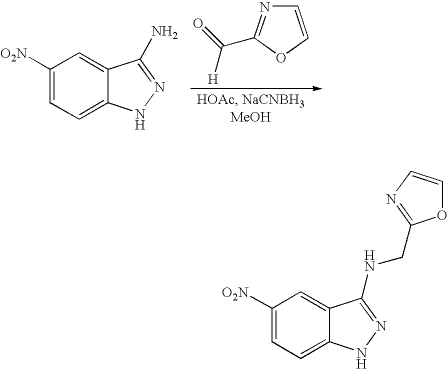

### (5-Nitro-1H-indazol-3-yl)-oxazol-2-ylmethyl-amine

To a suspension of 5-Nitro-1H-indazol-3-ylamine (100 mg, 0.56 mmol) in MeOH (15 mL) was added HOAc (0.3 mL) and Oxazole-2-carbaldehyde (65 mg, 0.67 mmol). The mixture was stirred for 10 min. then NaCNBH₃ (106 mg, 1.68 mmol) was added. The reaction was allowed to stir 5 hr. then concentrated in vacuo. The residue was diluted with ethyl acetate (25 mL) and washed with NaHCO₃ (25 mL). The organic layer was dried over anhydrous sodium sulfate and concentrated in vacuo. Chromatography on silica yielded the title compound (45 mg, 31%). m/z (M+H)=260.23.

### EXAMPLE 67

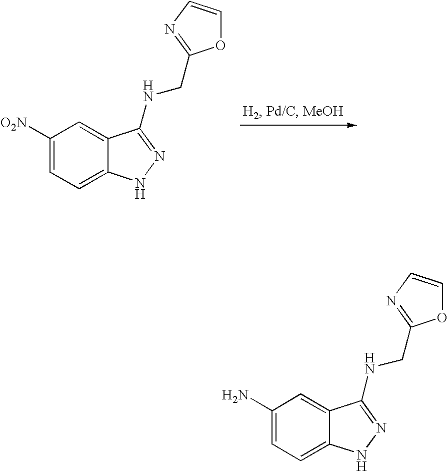

### N³-Oxazol-2-ylmethyl-1H-indazole-3,5-diamine

A solution of (5-Nitro-1H-indazol-3-yl)-oxazol-2-ylmethyl-amine (40 mg, 0.15 mmol) and 10% Pd/C (40 mg) in MeOH (10 mL) was hydrogenated at atmospheric pressure for 5 hr. The mixture was filtered through celite and the filtrate was concentrated to yield the title compound (27 mg, 77%). m/z (M+H)=230.24

### EXAMPLE 68

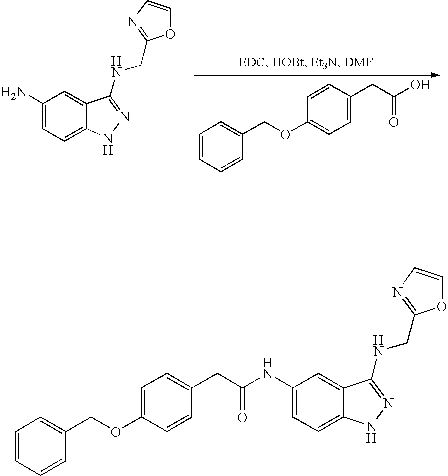

### 2-(4-Benzyloxy-phenyl)-N-[3-[(oxazol-2-ylmethyl)-amino]-1H-indazol-5-yl]-acetamide

To a solution of 4-phenoxyphenylacetic acid (31 mg, 0.13 mmol) in anhydrous DMF (5 mL) was added EDC (28 mg, 0.14 mmol) and HOBt (19 mg, 0.14 mmol). The mixture was stirred for 10 min. then Et₃N (0.030 mL, 0.20 mmol) and 3 N³-Oxazol-2-ylmethyl-1H-indazole-3,5-diamine (27 mg, 0.12 mmol) were added. The mixture was stirred at room temperature for 4 hr. then diluted with ethyl acetate (25 mL) and washed with aqueous sodium bicarbonate (25 mL). The aqueous layer was extracted with ethyl acetate (20 mL) and the combined organic layers were dried over anhydrous sodium sulfate and concentrated in vacuo. Chromatography on silica yielded the title compound (8 mg, 15%). m/z (M+H)=454.50.

### EXAMPLE 69

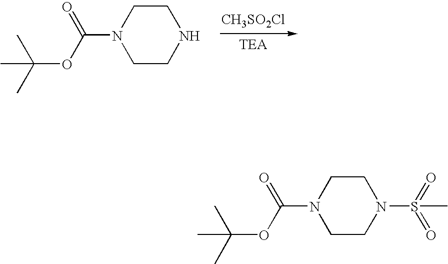

### tert-Butyl 4-(methylsulfonyl)piperazinecarboxylate

A solution of methanesulfonyl chloride (2.3 mL, 30 mmol) in 5 mL of dichloromethane was slowly added to cooled (0-5° C.) solution of tert-butyl piperazinecarboxylate (5.58 g, 30 mmol) in 60 mL of dichloromethane and the resulting mixture allowed to stir at room temperature for 1 hr. The reaction mixture was poured into 150 mL of water, and the organic layer was separated and washed with water (50 mL), 1M HCl (25 mL), water (50 mL), brine (25 mL). The combined organics were dried over anhydrous sodium sulfate and concentrated in vacuo to yield 7.42 g (93.7%) of the title compound (7.42 g, 93.7%). m/z (M+H)=265.35.

### EXAMPLE 70

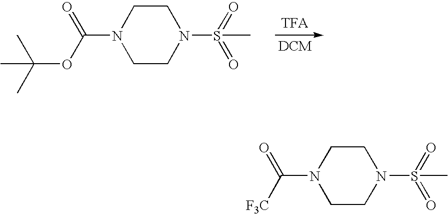

### 2,2,2-Trifluoro-1-[4-(methylsulfonyl)piperazin-1-yl]ethan-1-one

Trifluoroacetic acid (15 mL) was added to solution of tert-butyl 4-(methylsulfonyl)-piperazinecarboxylate (7.42 g, 28.1 mmol) in dichloromethane (100 mL) and stirred at room temperature for 2 hr. After evaporation to dryness in vacuo, the residue was dried at 60° C. in vacuo to afford the title compound (7.7.3 g, 100%). m/z (M+H)=261.24.

### EXAMPLE 71

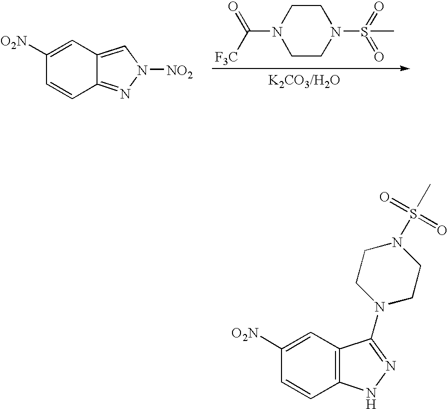

### 3-[(4-Methylsulfonyl)piperazin-1-yl]-5-nitro-1H-indazole

A mixture of 2,2,2-trifluoro-1-[4-(methylsulfonyl)piperazin-1-yl]ethan-1-one (0.65 g, 2.5 mmol), K₂CO₃ (0.345 g, 2.5 mmol) and water (10 mL) was stirred for 10 min, and 2,5-dinitro-2H-indazole (0.104 g, 0.5 mmol) was added. The resulting mixture was stirred for 2 hr. at 20° C. The product was then filtered, washed with water (20 mL), and dried to give the title compound (73 mg, 44.9%). m/z (M+H)=326.25. The material was used without further purification.

### EXAMPLE 72

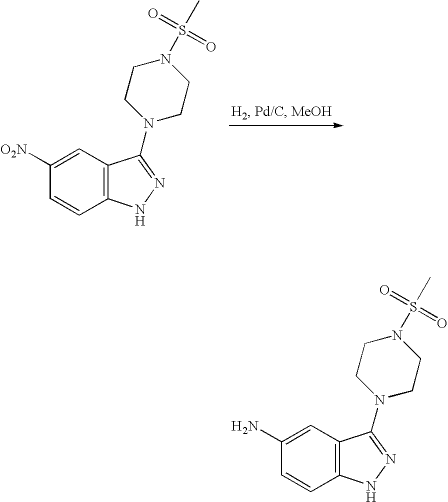

### 3-[(4-Methylsulfonyl)piperazin-1-yl]-1H-indazol-5-ylamine

A solution of 3-(4-Methylsulfonyl)piperazin-1-yl)-5-nitro-1H-indazole (73 mg, 0.22 mmol) and 10% Pd/C (200 mg) in MeOH (75 mL) was hydrogenated at atmospheric pressure for 2 hr. The mixture was filtered through celite and the filtrate was concentrated to yield the title compound (55 mg, 83%). m/z (M+H)=296.37.

### EXAMPLE 73

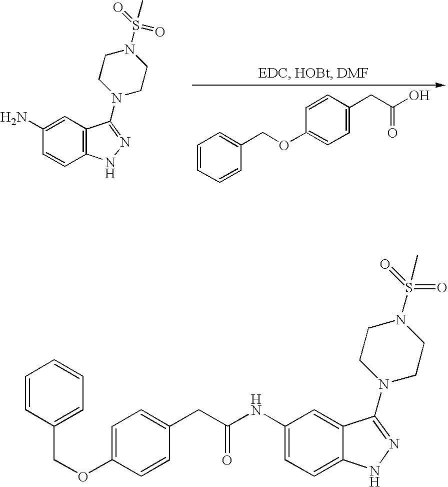

### 2-(4-Benzyloxy-phenyl)-N-{3-[(4-Methylsulfonyl)-piperazin-1-yl]-1H-indazol-5-yl}-acetamide

To a solution of 4-phenoxyphenylacetic acid (50 mg, 0.21 mmol) in anhydrous DMF (5 mL) was added EDC (44 mg, 0.23 mmol) and HOBt (31 mg, 0.23 mmol). The mixture was stirred for 10 min. then Et₃N (0.040 mL, 0.29 mmol) and 3-[(4-methylsulfonyl)piperazin-1-yl]-1H-indazol-5-ylamine (50 mg, 0.21 mmol) were added. The mixture was stirred at room temperature for 17 hr. then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (40 mg, 36.7%). m/z (M+H)=520.63.

### EXAMPLE 74

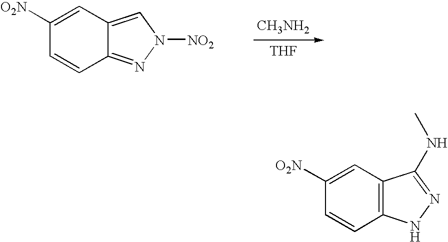

### 3-Methylamino-5-nitro-1H-indazole

To a solution of 2,5-dinitro-2H-indazole (624 mg g, 3.0 mmol) in THF (10 mL) was added 2M solution of methylamine in THF (2.5 mL, 5.0 mmol). The mixture was stirred for 15 min, then concentrated in vacuo. The residue was diluted with ethyl acetate (30 mL) and washed with sat. aq. sodium bicarbonate (15 mL). The aqueous layer was extracted with ethyl acetate (20 mL). The combined organic layers were dried over anhydrous sodium sulfate and concentrated in vacuo. Chromatography on silica yielded the title compound (430 mg g, 74.7%). m/z (M+H)=193.18.

### EXAMPLE 75

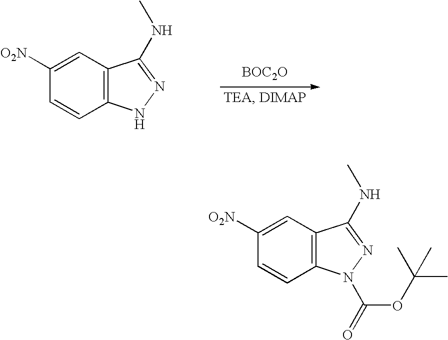

### tert-Butyl 3-methylamino-5-nitro-1H-indazolecarboxylate

A mixture of 3-methylamino-5-nitro-1H-indazole (250 mg, 1.30 mmol), BOC₂O (312 mg, 1.43 mmol), DIMAP (16 mg, 0.13 mmol), and TEA (0.217 mL, 1.56 mmol) in THF (5 mL) was stirred for 17 h at 20° C. The mixture was then poured in water (75 mL), and sat. aq. sodium bicarbonate (50 mL) was added. The product was extracted with EtOAc (3×50 mL). The combined organic layers were dried over anhydrous sodium sulfate and concentrated in vacuo. Chromatography on silica yielded the title compound (320 mg, 84.3%). m/z (M+H)=293.30.

### EXAMPLE 76

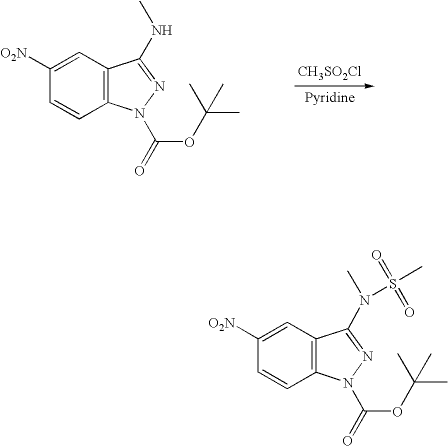

### tert-Butyl 3-[methyl(methylsulfonyl)amino]-5-nitro-1H-indazolecarboxylate

A mixture of tert-butyl 3-methylamino-5-nitro-1H-indazolecarboxylate (158 mg, 0.54 mmol), methanesulfonyl chloride (0.420 mL, 5.4 mmol) in pyridine (4 mL) was stirred for 17 h at 20° C. The resulting mixture was then poured in water (100 mL) and the product was extracted with EtOAc (3×50 mL). The combined organic layers were washed with 2M HCl (50 mL), water (50 mL), brine (25 mL), then dried over anhydrous sodium sulfate and concentrated in vacuo. Chromatography on silica yielded the title compound (138 mg, 69.1%). m/z (M+H)=371.39.

### EXAMPLE 77

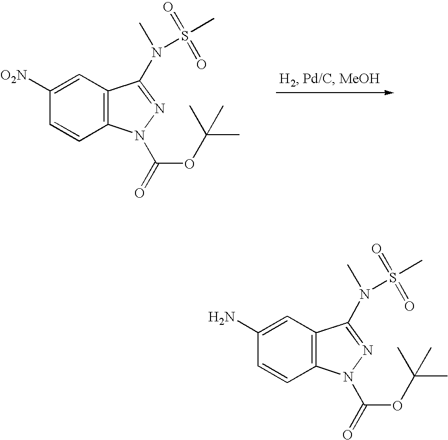

### tert-Butyl 5-amino-3-[methyl(methylsulfonyl)amino]-1H-indazolecarboxylate

A solution of tert-Butyl 3-[methyl(methylsulfonyl)amino]-5-nitro-1H-indazole carboxylate (138 mg, 0.37 mmol) and 10% Pd/C (200 mg) in MeOH (50 mL) was hydrogenated at atmospheric pressure for 3 hr. The mixture was filtered through celite and the filtrate was concentrated to yield the title compound (115 mg, 91.4%). m/z (M+H)=341.40.

### EXAMPLE 78

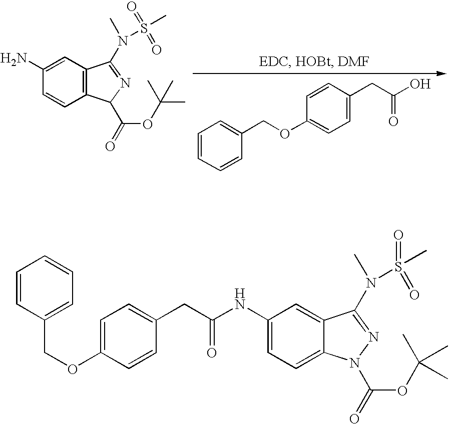

### tert-Butyl 3-[methyl(methylsulfonyl)amino]-5-{2-[4-(benzyloxy)phenyl]acetylamino}-1H-indazolecarboxylate

To a solution of 4-phenoxyphenylacetic acid (90 mg, 0.372 mmol) in anhydrous DMF (8 mL) was added EDC (78 mg, 0.406 mmol) and HOBt (55 mg, 0.406 mmol). The mixture was stirred for 10 min. then Et₃N (0.072 mL, 0.51 mmol) and tert-butyl 5-amino-3-[methyl(methylsulfonyl)amino]-1H-indazolecarboxylate (115 mg, 0.338 mmol) were added. The mixture was stirred at room temperature for 17 hr. then diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (78 mg, 41%). m/z (M+H)=565.67.

### EXAMPLE 79

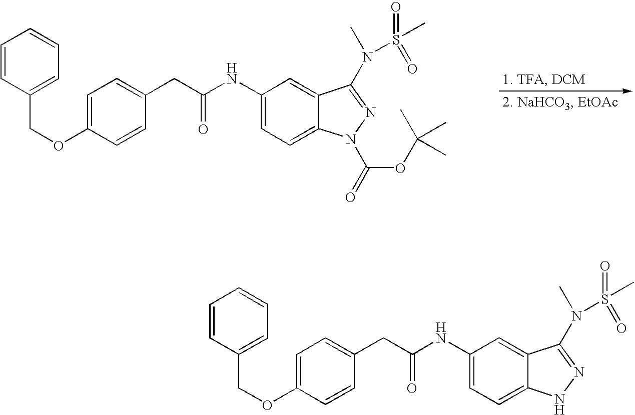

### N-{3-[Methyl(methylsulfonylamino](1H-indazol-5-yl)}-2-(4-Benzyloxy-phenyl)acetamide

tert-Butyl 3-[methyl(methylsulfonyl)amino]-5-{2-[4-(benzyloxy)phenyl]acetylamino}-1H-indazolecarboxylate (78 mg, 0.138 mmol) was dissolved in mixture of trifluoroacetic acid (TFA) (4 mL) and dichloromethane (DCM) (4 mL), and stirred for 30 min. The DCM and TFA were evaporated in vacuo, the rest was dissolved in EtOAc (50 mL), washed with sat. aq. sodium bicarbonate (30 mL), dried over anhydrous sodium sulfate and concentrated in vacuo. Chromatography on silica yielded the title compound (20 mg, 31.2%). m/z (M+H)=465.55.

### EXAMPLE 80

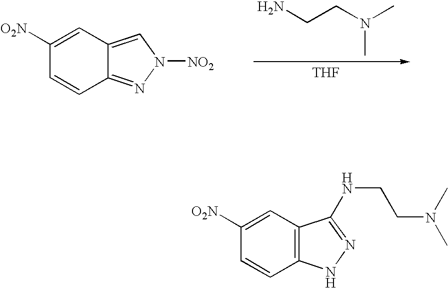

### N,N-Dimethyl-N′-(5-nitro-1H-indazol-3-yl)-ethane-1,3-diamine

To a solution of 2,5-dinitro-2H-indazole (312 mg g, 1.5 mmol) in THF (10 mL) was added N,N-dimethyl ethylenediamine (1.0 mL, 9.2 mmol). The mixture was stirred for 30 min, and then concentrated in vacuo. The residue was diluted with ethyl acetate (30 mL) and washed with sat. aq. sodium bicarbonate (15 mL). The aqueous layer was extracted with ethyl acetate (20 mL). The combined organic layers were dried over anhydrous sodium sulfate and concentrated in vacuo. Chromatography on silica yielded the title compound (160 mg, 42.8%). m/z (M+H)=250.27.

### EXAMPLE 81

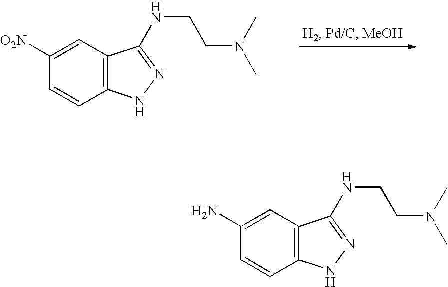

### N,N-Dimethyl-N′-(5-amino-1H-indazol-3-yl)-ethane-1,3-diamine

A solution of N,N-Dimethyl-N′-(5-nitro-1H-indazol-3-yl)-ethane-1,3-diamine (160 mg, 0.64 mmol) and 10% Pd/C (200 mg) in MeOH (100 mL) was hydrogenated at atmospheric pressure for 3 hr. The mixture was filtered through celite and the filtrate was concentrated to yield the title compound (140 mg, 100%). m/z (M+H)=219.30.

### EXAMPLE 82

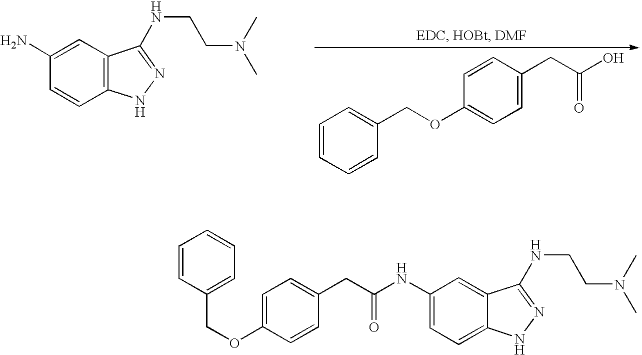

### N-(3-{[2-(Dimethylamino)ethyl]amino}(1H-indazol-5-yl)-2-(4-benzyloxy-phenyl)acetamide

To a solution of 4-phenoxyphenylacetic acid (180 mg, 0.74 mmol) in anhydrous DMF (8 mL) was added EDC (156 mg, 0.81 mmol) and HOBt (109 mg, 0.81 mmol). The mixture was stirred for 10 min. then Et₃N (0.140 mL, 1.0 mmol) and N,N-Dimethyl-N′-(5-amino-1H-indazol-3-yl)-ethane-1,3-diamine (147 mg, 0.67 mmol) were added. The mixture was stirred at room temperature for 2 hr. then diluted with ethyl acetate (50 mL) and washed with aqueous sodium bicarbonate (50 mL). The aqueous layer was extracted with ethyl acetate (50 mL) and the combined organic layers were dried over anhydrous sodium sulfate and concentrated in vacuo. Chromatography on silica yielded the title compound (107 mg, 36.0%). m/z (M+H)=444.55.

### EXAMPLE 83

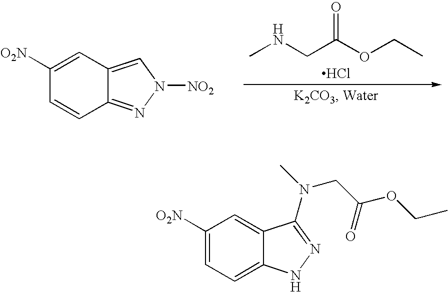

### [Methyl-(5-nitro-1H-indazol-3-yl)-amino]-acetic acid ethyl ester

To a solution of sarcosine methyl ester hydrochloride (3.70 g, 24.1 mmol) and potassium carbonate (3.33 g, 24.1 mmol) in water was added 2,5-dinitro-2H-indazole (1.0 g, 4.81 mmol). The mixture was allowed to stir for 2 hr. The suspension was filtered and dried to provide the title compound (752 mg, 1.34 g). m/z (M+H)=279.27.

### EXAMPLE 84

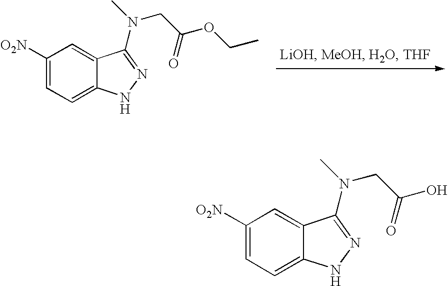

### [Methyl-(5-nitro-1H-indazol-3-yl)-amino]-acetic acid

To a solution of [Methyl-(5-nitro-1H-indazol-3-yl)-amino]-acetic acid ethyl ester (11.0 g, 3.61 mmol) in MeOH (10 mL), THF (10 mL) and water (5 mL) was added lithium hydroxide monohydrate (758 mg, 18.1 mmol). The mixture was stirred for 2 hr. then concentrated in vacuo. The residue was diluted with water (15 mL) and acidified with 2N HCl. The precipitate was filtered and dried to give the title compound (788 mg, 87%). m/z (M+H)=251.22.

### EXAMPLE 85

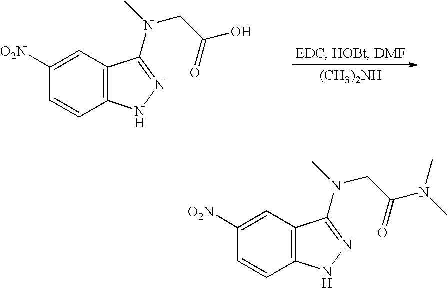

### N,N-Dimethyl-2-[methyl(5-nitro(1H-indazol-3-yl))amino]acetamide

To a solution of 2-[methyl(5-nitro(1H-indazol-3-yl))amino]acetic acid (100 mg, 0.40 mmol) in anhydrous DMF (5 mL) was added EDC (92 mg, 0.48 mmol) and HOBt (65 mg, 0.48 mmol). The mixture was stirred for 10 min. then Et₃N (0.083 mL, 0.60 mmol) and dimethylamine (2M solution in THF) (0.75 mL, 1.5 mmol) were added. The mixture was stirred at room temperature for 17 hr. then diluted with ethyl acetate (50 mL) and washed with aqueous sodium bicarbonate (50 mL). The aqueous layer was extracted with ethyl acetate (50 mL) and the combined organic layers were dried over anhydrous sodium sulfate and concentrated in vacuo. Chromatography on silica yielded the title compound (109 mg, 98.4%). m/z (M+H)=278.29.

### EXAMPLE 86

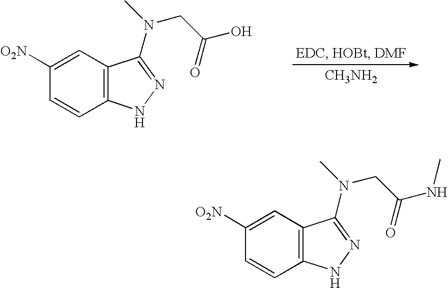

### N-Methyl-2-[methyl(5-nitro(1H-indazol-3-yl))amino]acetamide

To a solution of 2-[methyl(5-nitro(1H-indazol-3-yl))amino]acetic acid (100 mg, 0.40 mmol) in anhydrous DMF (5 mL) was added EDC (92 mg, 0.48 mmol) and HOBt (65 mg, 0.48 mmol). The mixture was stirred for 10 min. then Et₃N (0.083 mL, 0.60 mmol) and methylamine (2M solution in THF) (0.75 mL, 1.5 mmol) were added. The mixture was stirred at room temperature for 2 hr. then diluted with ethyl acetate (50 mL) and washed with aqueous sodium bicarbonate (50 mL). The aqueous layer was extracted with ethyl acetate (50 mL) and combined organic layers were dried over anhydrous sodium sulfate and concentrated in vacuo. Chromatography on silica yielded the title compound (87 mg, 82.7%). m/z (M+H)=264.25.

### EXAMPLE 87

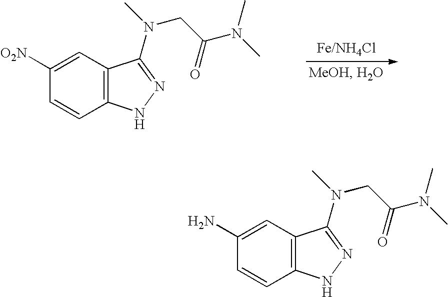

### N,N-Dimethyl-2-[methyl(5-amino(1H-indazol-3-yl))amino]acetamide

A mixture of N,N-Dimethyl-2-[methyl(5-nitro(1H-indazol-3-yl))amino]acetamide (109 mg, 0.394 mmol), Fe (221 mg, 3.94 mmol), NH₄Cl (11 mg, 0.197 mmol) in 20% aqueous MeOH (5 mL) was refluxed for 17 hr. Aqueous sodium bicarbonate (50 mL) was then added, and the product was extracted with EtOAc (3×50 mL). The combined organic layers were dried over anhydrous sodium sulfate and concentrated in vacuo. Chromatography on silica yielded the title compound (18 mg, 18.5%). m/z (M+H)=248.3.

### EXAMPLE 88

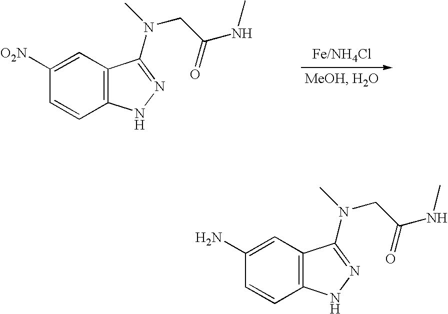

### N-Methyl-2-[methyl(5-amino(1H-indazol-3-yl))amino]acetamide

A mixture of N-methyl-2-[methyl(5-nitro(1H-indazol-3-yl))amino]acetamide (85 mg, 0.323 mmol), Fe (172 mg, 3.32 mmol), NH₄Cl (9 mg, 0.162 mmol) in 20% aqueous MeOH (5 mL) was refluxed for 17 h. Aqueous sodium bicarbonate (50 mL) was then added, and the product was extracted with EtOAc (3×50 mL). The combined organic layers were dried over anhydrous sodium sulfate and concentrated in vacuo. Chromatography on silica yielded the title compound (28 mg, 37.2%). m/z (M+H)=234.28.

### EXAMPLE 89

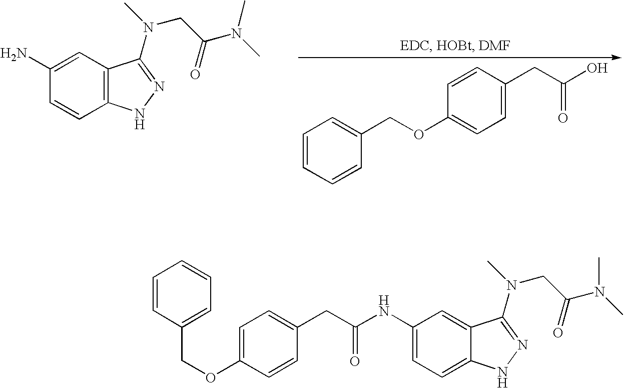

### N,N-Dimethyl-2-[methyl(5-[2-(4-benzyloxy-phenyl)acetylamino](1H-indazol-3-yl))amino]acetamide

To a solution of 4-phenoxyphenylacetic acid (18 mg, 0.073 mmol) in anhydrous DMF (2 mL) was added EDC (17 mg, 0.088 mmol) and HOBt (12 mg, 0.088 mmol). The mixture was stirred for 10 min. then Et₃N (0.016 mL, 0.132 mmol) and N,N-Dimethyl-2-[methyl(5-amino(1H-indazol-3-yl))amino]acetamide (20 mg, 0.08 mmol) were added. The mixture was stirred at room temperature for 4 hr. then diluted with ethyl acetate (50 mL) and washed with aqueous sodium bicarbonate (50 mL). The aqueous layer was extracted with ethyl acetate (50 mL) and the combined organic layers were dried over anhydrous sodium sulfate and concentrated in vacuo. Chromatography on silica yielded the title compound (19 mg, 55.3%). m/z (M+H)=472.56.

### EXAMPLE 90

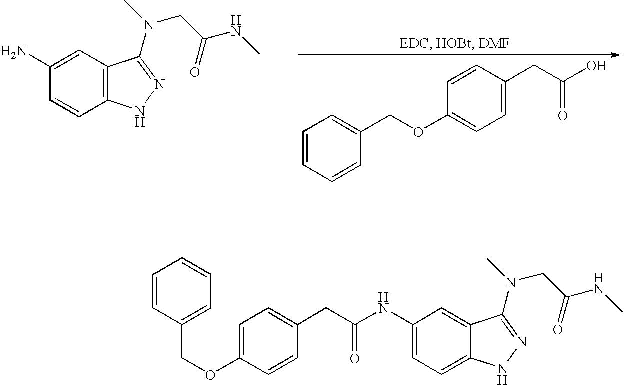

### N-Methyl-2-[methyl(5-[2-(4-benzyloxy-phenyl)acetylamino] (1H-indazol-3-yl))amino]acetamide

To a solution of 4-phenoxyphenylacetic acid (32 mg, 0.132 mmol) in anhydrous DMF (3 mL) was added EDC (28 mg, 0.144 mmol) and HOBt (20 mg, 0.144 mmol). The mixture was stirred for 10 min. then Et₃N (0.025 mL, 0.18 mmol) and N-methyl-2-[methyl(5-amino(1H-indazol-3-yl))amino]acetamide (28 mg, 0.12 mmol) were added. The mixture was stirred at room temperature for 4 hr. then diluted with ethyl acetate (50 mL) and washed with aqueous sodium bicarbonate (50 mL). The aqueous layer was extracted with ethyl acetate (50 mL) and the combined organic layers were dried over anhydrous sodium sulfate and concentrated in vacuo. Chromatography on silica yielded the title compound (30 mg, 54.7%). m/z (M+H)=458.54.

### EXAMPLE 91

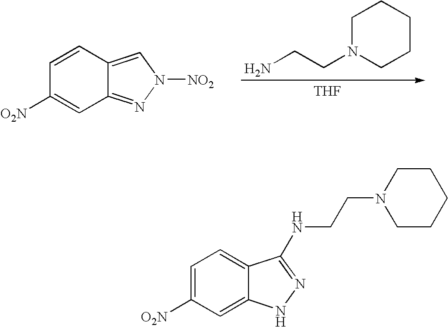

### (6-Nitro-1H-indazol-3-yl)-(2-piperidin-1-yl-ethyl)-amine

2-Piperidin-1-yl-ethylamine (2.6 mL, 18.5 mmol) in THF (10 mL) was added to a solution of 2,6-dinitro-2H-indazole (1.80 g, 11.0 mmol) in THF (65 mL). The mixture was stirred for 15 min, and then concentrated in vacuo. The residue was diluted with ethyl acetate (150 mL) and washed with sat. aq. sodium bicarbonate (50 mL). The aqueous layer was extracted with ethyl acetate (50 mL). The combined organic layers were dried over anhydrous sodium sulfate and concentrated in vacuo. Chromatography on silica yielded the title compound (1.61 g, 50.6%). m/z (M+H)=290.34.

### EXAMPLE 92

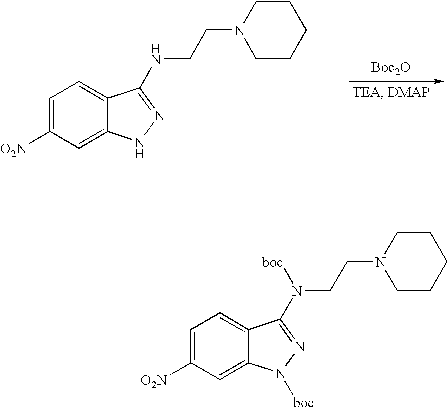

### 3-[tert-Butoxycarbonyl-(2-piperidin-1-yl-ethyl)-amino]-6-nitro-1H-indazole-1-carboxylic acid tert-butyl ester

A mixture of (6-Nitro-1H-indazol-3-yl)-(2-piperidin-1-yl-ethyl)-amine (1.61 g, 5.57 mmol), BOC₂O (3.04 g, 13.9 mmol), DMAP (68 mg, 0.56 mmol), and TEA (1.55 mL, 11.14 mmol) in THF (75 mL) was stirred for 17 h at 20° C. The mixture was poured into water (300 mL), sat. aq. sodium bicarbonate (200 mL) was added, and the product extracted with ethyl acetate (3×150 mL). The combined organic layers were dried over anhydrous sodium sulfate and concentrated in vacuo. Chromatography on silica yielded the title compound (1.26 g, 46.3%). m/z (M+H)=490.58

### EXAMPLE 93

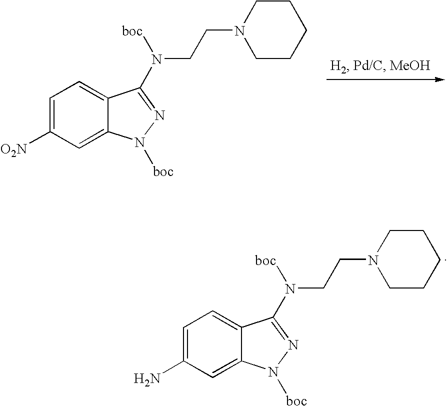

### 6-Amino-3-[tert-butoxycarbonyl-(2-piperidin-1-yl-ethyl)-amino]-1H-indazole-1-carboxylic acid tert-butyl ester

A solution of 3-[tert-Butoxycarbonyl-(2-piperidin-1-yl-ethyl)-amino]-6-nitro-1H-indazole-1-carboxylic acid tert-butyl ester (1.26 g, 2.58 mmol) and 10% Pd/C (250 mg) in MeOH (100 mL) was hydrogenated at atmospheric pressure for 4 h. The mixture was filtered through celite and the filtrate was concentrated to yield the title compound (1.09 g, 92.2%). m/z (M+H)=460.59.

### EXAMPLE 94

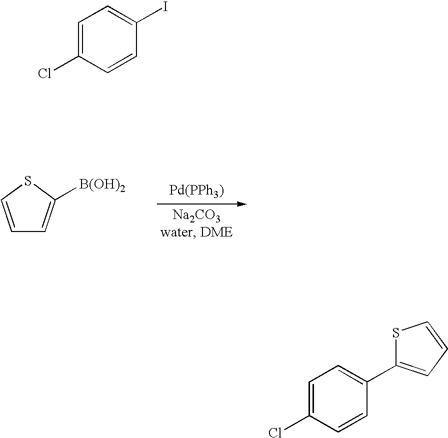

### 2-(4-Chloro-phenyl)-thiophene

A mixture of 4-chloroiodobenzene (3.0 g, 12.0 mmol), thiophene-2-boronic acid (1.94 g, 15.1 mmol), tetakistriphenylphosphine palladium (0) (728 mg, 0.63 mmol) and sodium carbonate (2.67 g, 25.2 mmol) in water (15 mL) and DME (15 mL) was heated at reflux for 4 h. The mixture was cooled and diluted with ethyl acetate (50 mL) and washed with sat. aq. sodium bicarbonate (50 mL). The aqueous layer was extracted with ethyl acetate (25 mL) and the combined organic layers were dried over anhydrous sodium sulfate and concentrated. Chromatography on silica (100% hexanes) yielded the title compound (1.97 g, 80%).

### EXAMPLE 95

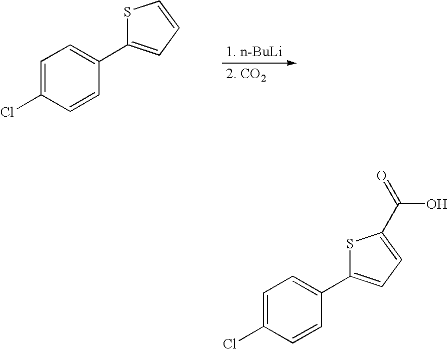

### 5-(4-Chloro-phenyl)-thiophene-2-carboxylic acid

A solution of 4-chloroiodobenzene (400 mg, 2.05 mmol) in anhydrous THF under an atmosphere of nitrogen was cooled to −78° C. A solution of n-BuLi (1.40 mL, 2.26 mmol, 1.6M in hexanes) was added. The mixture was stirred for 10 min. then poured onto freshly crushed dry ice and stirred for 1 h. The mixture was concentrated and dissolved in water (25 mL) and acidified with 2N HCl. The resulting precipitate was filtered and dried to yield the title compound (320 mg, 66%).

### EXAMPLE 96

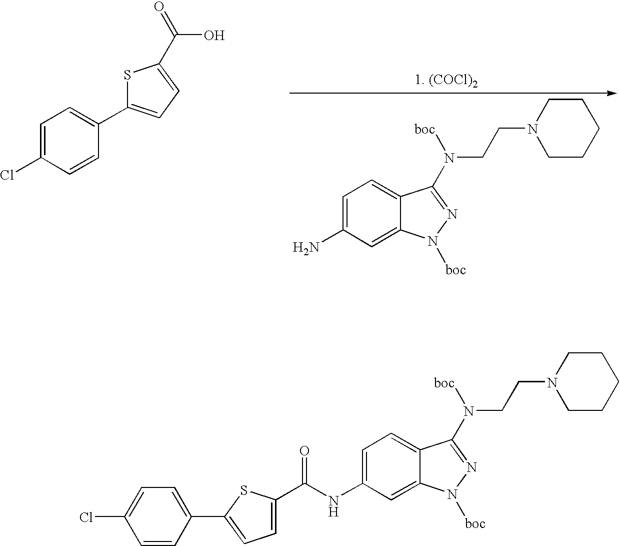

### 3-[tert-Butoxycarbonyl-(2-piperidin-1-yl-ethyl)-amino]-6-{[5-(4-chloro-phenyl)-thiophene-2-carbonyl]-amino}-3a,7a-dihydro-indazole-1-carboxylic acid tert-butyl ester

Oxalyl Chloride (0.07 mL, 0.81 mmol) was added to a suspension of 5-(4-chlorophenyl)-thiophene-2-carboxylic acid (128 mg, 0.81 mmol) in dichloromethane (5 mL) and stirred for 30 min at 20° C. The dichloromethane and excess of oxalyl chloride were removed in vacuo. THF (5 mL) was then added, followed by the addition of TEA (0.126 M, 0.9 mmol) and 6-amino-3-[tert-butoxycarbonyl-(2-piperidin-1-yl-ethyl)-amino]-1H-indazole-1-carboxylic acid tert-butyl ester (165 mg, 0.36 mmol). The mixture was stirred at room temperature for 17 h, diluted with ethyl acetate (50 mL) and washed with aqueous sodium bicarbonate (25 mL). The aqueous layer was extracted with ethyl acetate (50 mL) and the combined organic layers were dried over anhydrous sodium sulfate and concentrated in vacuo. Chromatography on silica yielded the title compound (183 mg, 75.0%). m/z (M+H)=680.27.

### EXAMPLE 97

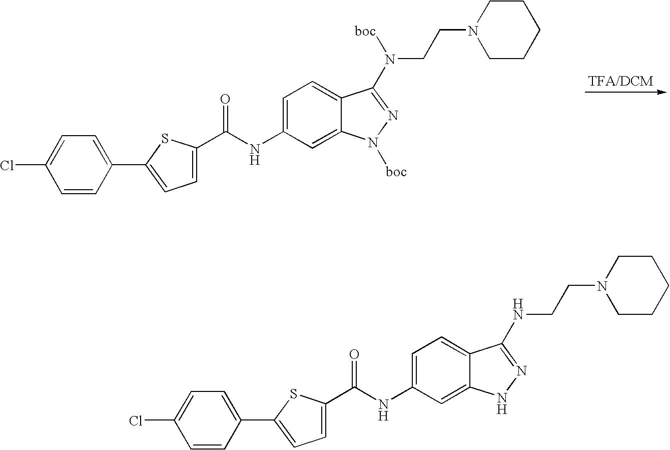

### 5-(4-Chloro-phenyl)-thiophene-2-carboxylic acid [3-(2-piperidin-1-yl-ethylamino)-3a,7a-dihydro-1H-indazol-6-yl]-amide

A mixture of 3-[tert-butoxycarbonyl-(2-piperidin-1-yl-ethyl)-amino]-6-{[5-(4-chloro-phenyl)-thiophene-2-carbonyl]-amino}-3a,7a-dihydro-indazole-1-carboxylic acid tert-butyl ester (183 mg, 0.27 mmol), TFA (2 mL) in dichloromethane (10 mL) was stirred for 17 h at 20° C. The dichloromethane and excess of TFA were removed in vacuo. The residue was dissolved in ethyl acetate (50 mL) and washed with aqueous sodium bicarbonate (25 mL). The aqueous layer was extracted with ethyl acetate (30 mL) and the combined organic layers were dried over anhydrous sodium sulfate and concentrated in vacuo. Chromatography on silica yielded the title compound (130 mg, 100%). m/z (M+H)=480.04.

### EXAMPLE 98

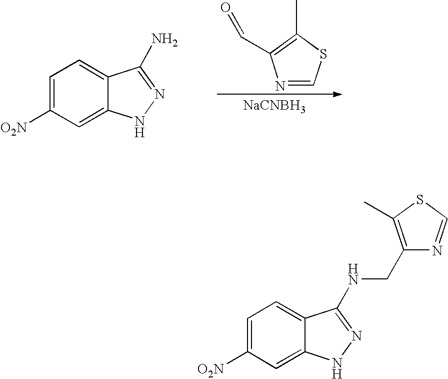

### (5-Methyl-thiazol-4-ylmethyl)-(6-nitro-1H-indazol-3-yl)-amine

A solution of 6-nitro-1H-indazol-3-ylamine (260 mg, 1.46 mmol), 5-Methyl-thiazole-4-carbaldehyde (223 mg, 1.75 mmol), acetic acid (0.10 mL) in methanol was stirred at ambient temperature for 10 min. Sodium cyano borohydride (110 mg, 1.75 mL) was added and the mixture was stirred for 15 h. The precipitate was filtered and dried to yield the title compound (160 mg, 38%). m/z (M+H)=290.13.

### EXAMPLE 99

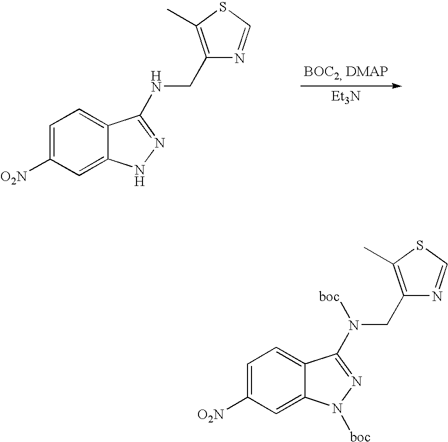

### 3-[tert-Butoxycarbonyl-(5-methyl-thiazol-4-ylmethyl)-amino]-6-nitro-indazole-1-carboxylic acid tert-butyl ester

To a solution of (5-methyl-thiazol-4-ylmethyl)-(6-nitro-1H-indazol-3-yl)-amine (160 mg, 0.55 mmol), DMAP (10 mg, 0.055 mmol) and triethylamine (1.7 mL, 1.22 mmol) in dry THF (10 mL) was added boc-anhydride (267 mg, 1.22 mmol). The mixture was stirred for 2 h and diluted with ethyl acetate (30 mL) and washed with water (25 mL). The organic layer was dried and concentrated. Chromatography on silica (100% hexanes to 50% hexanes/ethyl acetate) afforded the title compound (93 mg, 35%). m/z (M+H)=490.55.

### EXAMPLE 100

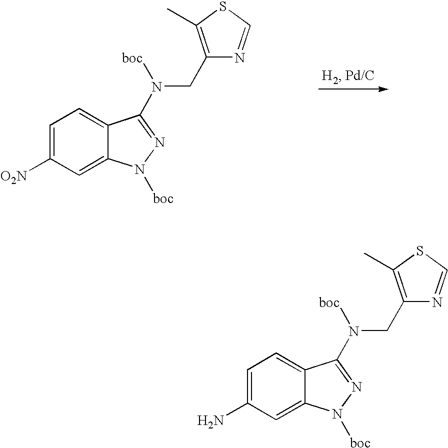

### 6-Amino-3-[tert-butoxycarbonyl-(5-methyl-thiazol-4-ylmethyl)-amino]-indazole-1-carboxylic acid tert-butyl ester

A mixture of 3-[tert-butoxycarbonyl-(5-methyl-thiazol-4-ylmethyl)-amino]-6-nitro-indazole-1-carboxylic acid tert-butyl ester (93 mg, 0.19 mmol) and 10% Pd on carbon in methanol (10 mL) was stirred under a balloon filled with hydrogen for 15 h. The mixture was filtered through celite and concentrated to yield the title compound (80 mg, 92%). m/z (M+H)=460.50.

### EXAMPLE 101

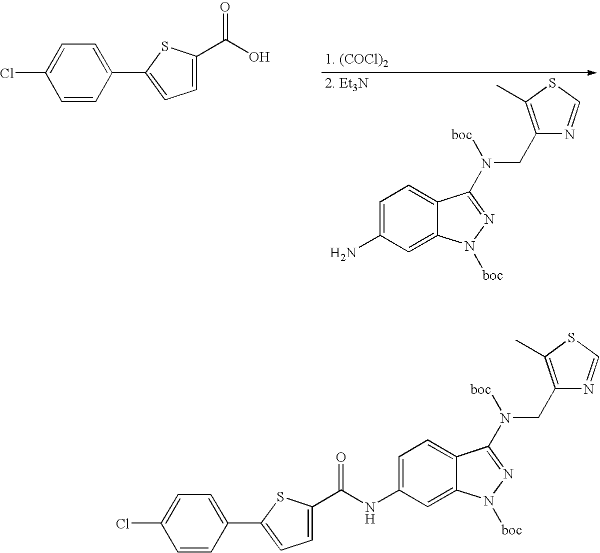

### 3-[tert-Butoxycarbonyl-(5-methyl-thiazol-4-ylmethyl)-amino]-6-{[5-(4-chloro-phenyl)-thiophene-2-carbonyl]-amino}-indazole-1-carboxylic acid tert-butyl ester

To a solution of 5-(4-chloro-phenyl)-thiophene-2-carboxylic acid (83 mg, 0.35 mmol) in dichloromethane (10 mL) was added oxalyl chloride (0.06 mL, 0.68 mmol) and DMF (0.01 mL). The solution was stirred for 1 h then concentrated. The residue was diluted with dichloromethane (5 mL) and added to a solution of 6-amino-3-[tert-butoxycarbonyl-(5-methyl-thiazol-4-ylmethyl)-amino]-indazole-1-carboxylic acid tert-butyl ester (80 mg, 0.17 mmol) and triethylamine (0.095 mL, 0.68 mmol) in dichloromethane. The mixture was stirred for 1 h then washed with sat. aq. sodium bicarbonate (15 mL). The organic layer was dried over anhydrous sodium sulfate and concentrated. Chromatography on silica (100% hexanes to 50% ethyl acetate/hexanes) yielded the title compound (55 mg, 47%). m/z (M+H)=680.36

### EXAMPLE 102

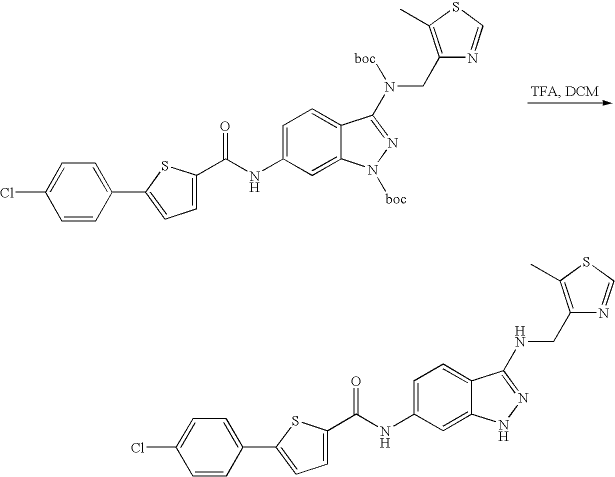

### 5-(4-Chloro-phenyl)-thiophene-2-carboxylic acid {3-[(5-methyl-thiazol-4-ylmethyl)-amino]-1H-indazol-6-yl}-amide

A solution of 3-[tert-butoxycarbonyl-(5-methyl-thiazol-4-ylmethyl)-amino]-6-{[5-(4-chloro-phenyl)-thiophene-2-carbonyl]-amino}-indazole-1-carboxylic acid tert-butyl ester (50 mg, 0.074 mmol), TFA (5 mL) in dichloromethane (10 mL) was stirred for 3 h. After this time the solution was concentrated and dissolved in methanol (2 mL). Saturated aqueous sodium bicarbonate (10 mL) was added and the mixture was stirred for 30 min. The resulting precipitate was then filtered to yield the title compound (20 mg, 57%). m/z (M+H)=480.11.

### Receptor Ligand Binding

The specific ligand binding to the receptors is defined as the difference between the total binding and the non-specific binding determined in the presence of an excess of unlabelled ligand. The results are expressed as a percent of control specific binding and as a percent inhibition of control specific binding obtained in the presence of the test compounds. The IC₅₀ value (concentration causing a half-maximal inhibition of control specific binding) and Hill coefficient (n_{H}) are determined by non-linear regression analysis of the competition curve using Hill equation curve fitting. The inhibition constant (Kᵢ) is calculated from the Cheng Prusoff equation (Kᵢ=IC₅₀/(1+(L/K_{D})) where L=concentration of radioligand ([¹²⁵I][Phe¹³]Tyr¹⁹-MCH) in the assay, and K_{D}=affinity of the radioligand for the receptor).

The compounds of the present invention typically show binding affinities of >50% at 20 μM concentration. For example, examples 51, 52, 53, 82, 97 and 102 have binding affinities of >70% at 10 μM concentration.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

All references cited herein are hereby incorporated by reference in their entirety, except where stated otherwise.

1-48. (canceled)

49. A compound of formula I: 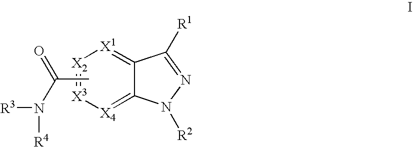
wherein
X¹ to X⁴ are each, independently, N, CH, CR, or C—, wherein C— represents the point of attachment of group —C(O)NR³R⁴;
R is alkyl, alkenyl, alkynyl, cycloalkyl, alkylcycloalkyl, aryl, alkylaryl, heteroaryl, alkylheteroaryl, heterocycle, alkylheterocycle, halogen, hydroxyl, cyano, alkoxy, arylaoxy, cycloalkyloxy, alkoxycarbonyl, carboxyl, amino, alkylamino, dialkylamino, —SH, thioalkyl, alkylsulfonyl, alkylsulfinyl, arylsulfonyl, arylsulfinyl, aminosulfonyl, aminosulfinyl, acyl or aroyl;
R¹ is heterocycle, heteroaryl, or NR^{a}R^{b} where
R^{a} and R^{b} are independently H, alkyl, alkenyl, alkynyl, aminoalkyl, amidoalkyl, cycloalkyl, alkylcycloalkyl, aryl, alkylaryl, heteroaryl, alkylheteroaryl, heterocycle, alkylheterocycle, alkylsulfonyl, or arylsulfonyl, with the proviso that R^{a} and R^{b} and not both H,
R² is H, alkyl, alkenyl, alkynyl, cycloalkyl, alkylcycloalkyl, aryl, alkylaryl, heteroaryl, alkylheteroaryl, heterocycle, alkylheterocycle, aroyl or acyl;
R³ is H, alkyl, alkenyl, alkynyl, cycloalkyl, alkylcycloalkyl, aryl, alkylaryl, heteroaryl, alkylheteroaryl, heterocycle, or alkylheterocycle;
R⁴ is aryl, heteroaryl, heterocycle, alkylaryl, alkylheteroaryl, alkylheterocycle, aryl-X, heteroaryl-X, heterocycle-X, alkylaryl-X, alkylheteroaryl-X, or alkylheterocycle-X; wherein X is Y-alkyl, Y-aryl, Y-alkylaryl, Y-heteoraryl, Y-alkylheteoraryl, Y-heterocycle, or Y-alkylheterocycle, and Y is —O—, —NR⁹—, or —(CO)—;
and pharmaceutically acceptable salts or solvates, or solvates of pharmaceutically acceptable salts thereof,
with the provisos that:
(i) R¹ is not substituted or unsubstituted benzimidazolyl, benzofuranyl or benzothienyl;
(ii) X¹ and X³ are not both N when the —C(O)NR³R⁴ group is attached at the 7-position; and
(iii) said compound is not:
N-1-azabicycle[2.2.2]oct-3-yl-3-(3-thienyl)-1H-indazole-6 carboxamide,
3-(2-quinolinyl)-N-tetrahydro-2-oxo-3-furanyl-1H-indazole-5-carboxamide,
N-(1,3-bendioxol-5-ylmethyl)-3-(3-pyridinyl)-1H-indazole-5-carboxamide,
N-((3,4-dihydro-2H-1-benzopyran-2-yl)methyl)-3-(2-pyridinyl)-1H-indazole-5-carbosamide,
3-(5-acetyl-2-thienyl)-N-(3-pyridinylmethyl)-1H-indazole-5-carboxamide,
N-[2-(1H-indol-3yl)ethyl]-3 (4-quinolyl)-1H-indazole-5-carboxamide, or
N-[[4-(methylsulfonyl)phenyl]methyl]-3-(2-quinolinyl)-1H-indazole-5-carboxamide,
or a pharmaceutically acceptable salt thereof.

50. A compound according to claim 49, wherein the —C(O)NR³R⁴ group is attached at the 5-position or the 6-position.

51. A compound according to claim 49, wherein each of X¹ to X⁴ that is not substituted by the —C(O)NR³R⁴ group is CH.

52. A compound according to claim 49, wherein R¹ is a 5- or 6-membered heterocycle, a 5- or 6-membered heteroaryl, or NR^{a}R^{b}.

53. A compound according to claim 49, wherein R¹ is substituted or unsubstituted oxazolyl, imidazolyl, piperazinyl, homopiperazinyl, or NR^{a}R^{b}.

54. A compound according to claim 49, wherein R¹ is substituted or unsubstituted piperazinyl or —NR^{a}R^{b}.

55. A compound according to claim 49, wherein R^{a} and R^{b} are independently H, alkyl aminoalkyl, amidoalkyl, alkylheteroaryl, alkylheterocycle or alkylsulfonyl.

56. A compound according to claim 49, wherein R¹ is —NHCH₂CH₂NMe₂, —N(CH₃)CH₂CH₂NMe₂, —NHCH₂CH₂(piperidine), —NHCH₂CH₂CH₂NMe₂, —N(CH₃)CH₂C(O)NHMe, —N(CH₃)CH₂C(O)NMe₂, 4-methylpiperazinyl, 4-methylhomopiperazinyl, 4-methylsulfonylpiperazinyl, oxazolyl, imidazolyl, oxazolylmethyl or thiazolylmethyl.

57. A compound according to claim 49, wherein R⁴ is aryl, alkylaryl, aryl-X or alkylaryl-X where X is Y-alkylaryl.

58. A compound according to claim 57, wherein R⁴ is biphenyl, optionally substituted benzyl, aryl-O-alkylaryl, or alkylaryl-O-alkylaryl.

59. A compound according to claim 58, wherein R⁴ is biphenyl, chlorobenzyl, methoxybenzyl, trifluoromethylbenzyl, dichlorobenzyl, trifluoromethoxybenzyl, benzyloxyphenyl, or benzyloxybenzyl.

60. A compound according to claim 49, wherein R¹ is heterocycle, heteroaryl other than benzimidazolyl, benzofuranyl, benzothienyl, pyridinyl or quinolinyl, or NR^{a}R^{b};
X¹-X⁴ are CH, CR or C— wherein C— represent the point of attachment of group —C(O)NR³R⁴; and
R⁴ is aryl, heteroaryl, alkylaryl, aryl-X, heteroaryl-X, heterocycle-X, alkylaryl-X, alkylheteroaryl-X, or alkylheterocycle-X.

61. A compound of formula I: 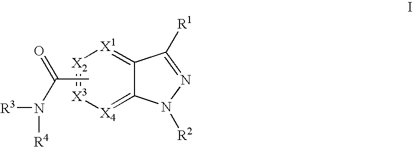
wherein
X¹ and X⁴ are CH;
X² and X³ are each, independently, CH or C—, wherein C— represents the point of attachment of group —(CO)NR³R⁴;
R¹ is heterocycle or NR^{a}R^{b} where
R^{a} and R^{b} are independently H, alkyl or aminoalkyl, with the proviso that R^{a} and R^{b} are not both H;
R² is H or alkyl;
R³ is H;
R⁴ is aryl, alkylaryl, or aryl-X, where X is Y-alkylaryl and Y is —O—;
and pharmaceutically acceptable salts or solvates, or solvates of pharmaceutically acceptable salts thereof.

62. A compound according to claim 49, chosen from:
3-(4-Methyl-piperazin-1-yl)-1H-indazole-6-carboxylic acid 4-chloro-benzylamide,
3-(4-Methyl-piperazin-1-yl)-1H-indazole-6-carboxylic acid benzylamide,
3-(4-Methyl-piperazin-1-yl)-1H-indazole-6-carboxylic acid 2-methoxy-benzylamide,
3-(4-Methyl-piperazin-1-yl)-1H-indazole-6-carboxylic acid 4-methoxy-benzylamide,
3-(4-Methyl-piperazin-1-yl)-1H-indazole-6-carboxylic acid 4-trifluoromethyl-benzylamide,
3-(4-Methyl-piperazin-1-yl)-1H-indazole-6-carboxylic acid 2,4-dichloro-benzylamide,
3-(4-Methyl-piperazin-1-yl)-1H-indazole-6-carboxylic acid 4-trifluoromethoxy-benzylamide,
3-(4-Methyl-piperazin-1-yl)-1H-indazole-6-carboxylic acid biphenyl-4-ylamide,
3-(4-Methyl-piperazin-1-yl)-1H-indazole-6-carboxylic acid (4-benzyloxy-phenyl)-amide,
1-Methyl-3-(4-methyl-piperazin-1-yl)-1H-indazole-6-carboxylic acid benzylamide,
1-Methyl-3-(4-methyl-piperazin-1-yl)-1H-indazole-6-carboxylic acid 4-trifluoromethyl-benzylamide,
1-Methyl-3-(4-methyl-piperazin-1-yl)-1H-indazole-6-carboxylic acid 2,4-dichloro-benzylamide,
1-Methyl-3-(4-methyl-piperazin-1-yl)-1H-indazole-6-carboxylic acid 4-methoxy-benzylamide,
1-Methyl-3-(4-methyl-piperazin-1-yl)-1H-indazole-6-carboxylic acid 2-methoxy-benzylamide,
4-Benzyloxy-phenyl)-[1-methyl-3-(4-methyl-piperazin-1-yl)-1H-indazol-6-yl]-methanone,
1-Methyl-3-(4-methyl-piperazin-1-yl)-1H-indazole-6-carboxylic acid 4-trifluoromethoxy-benzylamide,
3-[(2-Dimethylamino-ethyl)-methyl-amino]-1H-indazole-6-carboxylic acid 4-chloro-benzylamide,
3-[(2-Dimethylamino-ethyl)-methyl-amino]-1H-indazole-6-carboxylic acid 2,4-dichloro-benzylamide,
3-[(2-Dimethylamino-ethyl)-methyl-amino]-1H-indazole-6-carboxylic acid 4-trifluoromethyl-benzylamide,
3-[(2-Dimethylamino-ethyl)-methyl-amino]-1H-indazole-6-carboxylic acid 4-trifluoromethoxy-benzylamide,
3-[(2-Dimethylamino-ethyl)-methyl-amino]-1H-indazole-6-carboxylic acid (4-benzyloxy-phenyl)-amide,
3-(4-Methyl-piperazin-1-yl)-1H-indazole-5-carboxylic acid (4-benzyloxy-phenyl)-amide,
3-(4-Methyl-piperazin-1-yl)-1H-indazole-5-carboxylic acid biphenyl-4-ylamide,
3-(4-Methyl-piperazin-1-yl)-1H-indazole-5-carboxylic acid 4-chloro-benzylamide, and
1-Methyl-3-(4-methyl-piperazin-1-yl)-1H-indazole-5-carboxylic acid (4-benzyloxy-phenyl)-amide,
and pharmaceutically acceptable salts thereof, solvates thereof, and solvates of pharmaceutically acceptable salts thereof,
wherein if the compound exhibits chirality it can be in the form of a mixture of enantiomers such as a racemate or a mixture of diastereomers, or can be in the form of a single enantiomer or a single diastereomer.

63. A pharmaceutical composition comprising a compound of claim 49 and a pharmaceutically acceptable carrier.

64. A method for treating a condition that responds to a melanin concentrating hormone receptor ligand comprising administering to a patient in need thereof an effective amount of a compound according to claim 49.

65. The method of claim 64, wherein the condition is an eating disorder, weight gain, obesity, depression or anxiety.

66. The method of claim 65, wherein the condition is an eating disorder.

67. The method of claim 65, wherein the condition is obesity.

68. The method of claim 65 wherein the condition is weight gain.

69. A compound of formula II: 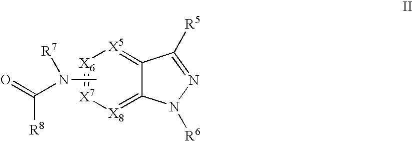
wherein
X⁵ to X⁸ are each, independently, N, CH, CR, or C—, wherein C— represents the point of attachment of group —NR⁷C(O)R⁸;
R is alkyl, alkenyl, alkynyl, cycloalkyl, alkylcycloalkyl, aryl, alkylaryl, heteroaryl, alkylheteroaryl, heterocycle, alkylheterocycle, halogen, hydroxyl, cyano, alkoxy, arylaoxy, cycloalkyloxy, alkoxycarbonyl, carboxyl, amino, alkylamino, dialkylamino, —SH, thioalkyl, alkylsulfonyl, alkylsulfinyl, arylsulfonyl, arylsulfinyl, aminosulfonyl, aminosulfinyl, acyl or aroyl;
R⁵ is heterocycle, heteroaryl, or NR^{c}R^{d} where
R^{c} and R^{d} are independently H, alkyl, alkenyl, alkynyl, aminoalkyl, alkylamido, cycloalkyl, alkylcycloalkyl, aryl, alkylaryl, heteroaryl, alkylheteroaryl, heterocycle, alkylheterocycle, alkylsulfonyl, or arylsulfonyl, with the proviso that R^{c} and R^{d} and not both H,
R⁶ is H, alkyl, alkenyl, alkynyl, cycloalkyl, alkylcycloalkyl, aryl, alkylaryl, heteroaryl, alkylheteroaryl, heterocycle, alkylheterocycle, aroyl or acyl;
R⁷ is H, alkyl, alkenyl, alkynyl, cycloalkyl, alkylcycloalkyl, aryl, alkylaryl, heteroaryl, alkylheteroaryl, heterocycle, or alkylheterocycle;
R⁸ is -D-E-F, where
D is alkylene, —NR¹⁰(aryl), —NR¹⁰(heteroaryl), alkylcycloalkyl, alkylaryl, or alkylheteoraryl;
E is —O—, —NR¹¹—, or —(CO)— and
F is alkylaryl, alkylheteroaryl or alkylheterocycle;
R⁹, R¹⁰ and R¹¹ are each, independently, H, alkyl, alkenyl, alkynyl, cycloalkyl, cycloalkylalkyl, aryl, alkylaryl heteroaryl, alkylheteroaryl, heterocycle, or alkylheterocycle;
and pharmaceutically acceptable salts or solvates, or solvates of pharmaceutically acceptable salts thereof.

70. A compound according to claim 69, wherein the —NR⁷C(O)R⁸ group is attached at the 5-position or the 6-position.

71. A compound according to claim 69, wherein each of X¹ to X⁴ that is not substituted by the —NR⁷C(O)R⁸ group is CH.

72. A compound according to claim 69, wherein R⁵ is a 5- or 6-membered heterocycle, a 5- or 6-membered heteroaryl, or NR^{c}R^{d}.

73. A compound according to claim 69, wherein R⁵ is substituted or unsubstituted oxazolyl, imidazolyl, piperazinyl, homopiperazinyl, or NR^{c}R^{d}.

74. A compound according to claim 69, wherein R^{c} and R^{d} are independently H, alkyl aminoalkyl, amidoalkyl, alkylheteroaryl, alkylheterocycle or alkylsulfonyl.

75. A compound according to claim 69, wherein R⁵ is —NHCH₂CH₂NMe₂, —N(CH₃)CH₂CH₂NMe₂, —NHCH₂CH₂(piperidine), —NHCH₂CH₂CH₂NMe₂, —N(CH₃)CH₂C(O)NHMe, —N(CH₃)CH₂C(O)NMe₂, 4-methylpiperazinyl, 4-methylhomopiperazinyl, 4-methylsulfonylpiperazinyl, oxazolyl, imidazolyl, oxazolylmethyl or thiazolylmethyl.

76. A compound according to claim 69, wherein
D is alkylene, —NR¹⁰(aryl), —NR¹⁰(heteroaryl), alkylcycloalkyl, alkylaryl, or alkylheteoraryl;
E is —O— or —NR¹¹—; and
F is alkylaryl, alkylheteroaryl or alkylheterocycle.

77. A compound according to claim 69, wherein
D is —NR¹⁰(aryl) or alkylaryl;
E is —O—; and
F is alkylaryl.

78. A compound according to claim 69, wherein R⁸ is —NR¹⁰(aryl)-Y-alkylaryl or alkylaryl-Y-alkylaryl.

79. A compound according to claim 78, wherein Y is O.

80. A compound according to claim 69, wherein
X⁵ and X⁸ are CH;
X⁶ to X⁷ are each, independently, CH or C—, wherein C— represents the point of attachment of group —NR⁷(CO)R⁸;
R⁵ is heterocycle, heteroaryl, or NR^{c}R^{d} where
R^{c} and R^{d} are independently H, alkyl, aminoalkyl, alkylamido, alkylcycloalkyl, alkylheteroaryl, alkylheterocycle or alkylsulfonyl, with the proviso that R^{c} and R^{d} and not both H,
R⁶ is H or alkyl;
R⁷ is H;
R⁸ is -D-E-F, where
D is —NR¹⁰(aryl) or alkylaryl;
E is —O—;
F is alkylaryl; and
R¹⁰ is H.

81. A compound according to claim 69, chosen from
2-(4-Benzyloxy-phenyl)-N-[3-(4-methyl-piperazin-1-yl)-1H-indazol-5-yl]-acetamide,
2-(4-Benzyloxy-phenyl)-N-{3-[(2-dimethylamino-ethyl)-methyl-amino]-1H-indazol-5-yl}-acetamide,
2-(4-Benzyloxy-phenyl)-N-[3-(4-methyl-piperazin-1-yl)-1H-indazol-6-yl]-acetamide,
2-(4-Benzyloxy-phenyl)-N-[3-(4-methyl-homopiperazin-1-yl)-1H-indazol-5-yl]-acetamide,
2-(4-Benzyloxy-phenyl)-N-[3-(2-Piperidin-1-yl-ethylamino)-1H-indazol-5-yl]-acetamide,
2-(4-Benzyloxy-phenyl)-N-[3-(3-dimethylaminopropylamino)-1H-indazol-5-yl]-acetamide,
{[3-(4-Methylpiperazinyl)(1H-indazol-6-yl]amino}-N-(4-phenoxyphenyl)carboxamide,
2-(4-Benzyloxy-phenyl)-N-(3-oxazol-2-yl-1H-indazol-5-yl)-acetamide,
2-(4-Benzyloxy-phenyl)-N-{3-[(thiazol-2-ylmethyl)-amino]-1H-indazol-5-yl}-acetamide
2-(4-Benzyloxy-phenyl)-N-(3-imidazol-1-yl-1H-indazol-5-yl)-acetamide,
2-(4-Benzyloxy-phenyl)-N-{3-[(oxazol-2-ylmethyl)-amino]-1H-indazol-5-yl}-acetamide,
2-(4-Benzyloxy-phenyl)-N-{3-[(4-Methylsulfonyl)-piperazin-1-yl]-1H-indazol-5-yl}-acetamide,
N-{3-[Methyl(methylsulfonylamino](1H-indazol-5-yl)}-2-(4-Benzyloxy-phenyl)acetamide,
N-(3-{[2-(Dimethylamino)ethyl]amino}(1H-indazol-5-yl)-2-(4-benzyloxy-phenyl)acetamide,
N,N-Dimethyl-2-[methyl(5-[2-(4-benzyloxy-phenyl)acetylamino](1H-indazol-3-yl))amino]acetamide, and
N-Methyl-2-[methyl(5-[2-(4-benzyloxy-phenyl)acetylamino](1H-indazol-3-yl))amino]acetamide,
and pharmaceutically acceptable salts thereof, solvates thereof, and solvates of pharmaceutically acceptable salts thereof,
wherein if the compound exhibits chirality it can be in the form of a mixture of enantiomers such as a racemate or a mixture of diastereomers, or can be in the form of a single enantiomer or a single diastereomer.

82. A pharmaceutical composition comprising a compound of claim 69 and a pharmaceutically acceptable carrier.

83. A method for treating a condition that responds to a melanin concentrating hormone receptor ligand comprising administering to a patient in need thereof an effective amount of a compound according to claim 69.

84. The method of claim 83, wherein the condition is an eating disorder, weight gain, obesity, depression or anxiety.

85. The method of claim 84, wherein the condition is an eating disorder.

86. The method of claim 84, wherein the condition is obesity.

87. The method of claim 84, wherein the condition is weight gain.

88. A compound of formula III: 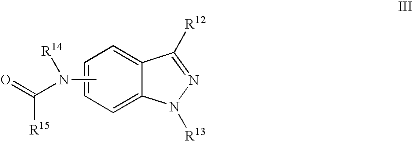
wherein
R¹² is NR^{e}R^{f} where R^{e} and R^{f} are each, independently, H, alkyl (e.g., methyl), alkylheterocycle or alkylheteraryl, with the proviso that R^{e} and R^{f} are not both H;
R¹³ and R¹⁴ are each independently H or alkyl (e.g., methyl); and
R¹⁵ is heteroaryl other than benzimidazolyl;
and pharmaceutically acceptable salts or solvates, or solvates of pharmaceutically acceptable salts thereof.

89. A compound according to claim 88, wherein R^{e} and R^{f} are each, independently, H, alkylheterocycle or alkylheteroaryl.

90. A compound according to claim 88, wherein R¹⁵ is a substituted or unsubstituted 5-membered heteroaryl.

91. A compound according to claim 88, selected from
5-(4-Chloro-phenyl)-thiophene-2-carboxylic acid [3-(2-piperidin-1-ylethylamino)-3a,7a-dihydro-1H-indazol-6-yl]-amide, and
5-(4-Chloro-phenyl)-thiophene-2-carboxylic acid {3-[(5-methyl-thiazol-4-ylmethyl)-amino]-1H-indazol-6-yl}-amide,
and pharmaceutically acceptable salts thereof, solvates thereof, and solvates of pharmaceutically acceptable salts thereof,
wherein if the compound exhibits chirality it can be in the form of a mixture of enantiomers such as a racemate or a mixture of diastereomers, or can be in the form of a single enantiomer or a single diastereomer.

92. A pharmaceutical composition comprising a compound of claim 88 and a pharmaceutically acceptable carrier.

93. A method for treating a condition that responds to a melanin concentrating hormone receptor ligand comprising administering to a patient in need thereof an effective amount of a compound according to claim 88.

94. The method of claim 93, wherein the condition is an eating disorder, weight gain, obesity, depression or anxiety.

95. The method of claim 94, wherein the condition is an eating disorder.

96. The method of claim 94, wherein the condition is obesity.

97. The method of claim 94, wherein the condition is weight gain.
